(19) United States
(12) Patent Application Publication
(10) Pub. No.: US 2007/0280957 A1
(43) Pub. Date: Dec. 6, 2007

(54) Change of the Load State of Mhc Molecules

(76) Inventors: Kirsten Falk, Berlin (DE); Olaf Rotzschke, Berlin (DE)

Correspondence Address: KLAUBER & JACKSON, 411 HACKENSACK AVENUE, HACKENSACK, NJ, 07601, US

(21) Appl. No.: 11/663,146

(22) PCT Filed: Sep. 16, 2005
(86) PCT No.: PCT/EP05/10008

§ 371 Date: Jul. 31, 2007

(30) Foreign Application Priority Data
Sep. 16, 2004 (DE) 10 2004 044 956 2
Nov. 11, 2004 (DE) 10 2004 054 545 6

(51) Int. Cl. C07K 1/113; A61K 38/00; G01N 33/00; G01N 33/566; A61K 39/385

(52) U.S. Cl. 424/193.1; 435/7.1; 435/7.92; 436/501; 514/2; 530/345

(57) ABSTRACT

The present invention relates to methods for changing the load state of MHC molecules with ligands, the change in the load state being catalysed by a compound of formulae I, IA, II, III or IV1 to IV3. The invention relates further to the use of compounds of formulae I, IA, II, III or IV1 to IV3 or to the use of MHC molecules loaded with ligands, which molecules can be prepared by a method according to the invention, for the treatment of disorders or conditions that are associated with various pathologically excessive or absent immune responses and also for triggering tumour-specific, pathogen-specific or autoreactive immune responses. The invention additionally relates to the use of such compounds for the treatment and diagnosis of cancer, infectious diseases, autoimmune diseases and for attenuating aggressive immune reactions, as well as to the preparation of a vaccine or of a pharmaceutical composition for the treatment of the mentioned disorders or conditions.

The present invention relates to methods for changing the load state of MHC molecules with ligands, the change in the load state being catalysed by a compound of formulae I, IA, II, III or IV1 to IV3. The invention relates further to the use of compounds of formulae I, IA, II, III or IV1 to IV3 or to the use of MHC molecules loaded with ligands, which molecules can be prepared by a method according to the invention, for the treatment of disorders or conditions that are associated with various pathologically excessive or absent immune responses and also for triggering tumour-specific, pathogen-specific or autoreactive immune responses. The invention additionally relates to the use of such compounds for the treatment and diagnosis of cancer, infectious diseases, autoimmune diseases and for attenuating aggressive immune reactions, as well as to the preparation of a vaccine or of a pharmaceutical composition for the treatment of the mentioned disorders or conditions.

The initiation of the cascade of the immune response proceeds substantially via MHC molecules and is based especially on the specific detection and repulsion of pathogenic invaders by the binding of major histocompatibility complex (MHC)-associated peptide antigens to T-cell receptors (TCR). The formation of stable MHC-peptide complexes is of central importance for the triggering of immune responses. Empty MHC-class II molecules are naturally unstable and decompose relatively quickly, while MHC-peptide complexes have a substantially longer half-life. The binding of CLIP or Ii (class II associated invariant chain peptide (CLIP/Ii₈₉₋₁₀₂)) immediately after expression of the MHC-class II molecules ensures inter alia that the complexes remain stable until they are loaded with antigenic peptides. The dissociation of bound peptides in particular at physiological pH values lasts for a very long time, however, and, depending on the particular peptide in question and on the MHC-class II allele, can last for several hours. Initial investigations found, however, that the low pH values present in late endosomal vesicles drastically accelerate the dissociation of CLIP and accordingly facilitate the association of other peptides (Avva, R. R., and P. Cresswell. 1994, Immunity 1:763-774). Nevertheless, the dissociation rates for many MHC alleles are still so high that the relatively short residence time in endosomal vesicles would not be sufficient to ensure the complete replacement of bonded CLIPs. A physiological cofactor is therefore necessary to catalyse the complete replacement. This is referred to as HLA-DM. HLA-DM is a transmembrane protein that is likewise coded for in the MHC-class II gene locus and accelerates the ligand replacement by binding to the peptide/MHC complex.

The major histocompatibility complex (MHC) gene locus controls a large number of important immunological functions. It is located on the short arm of chromosome 6 of the human genome and codes for several classes of MHC molecules. In humans, the major histocompatibility complex (MHC) is referred to as HLA (human leukocyte antigen) (Wake, C. T. 1986. Molecular biology of the HLA class I and class II genes. Mol Biol Med 3:1-11).

The molecules of class I are referred to as HLA-A, -B and -C. They describe a group of highly polymorphous membrane-located glycoproteins which serve to present endogenously expressed antigens at the cell surface. They are expressed in almost all cells of the human body and form heterodimers from the heavy MHC class I chains (HLA-A, -B or -C) and a small protein not coded for in the MHC gene locus, ββ2 microglobulin (β2m). The heavy α-chains possess a short cytoplasmic domain, a transmembrane domain and three extracellular domains that are linked non-covalently with β2m. The antigens bound by MHC class I are peptides having almost exculsively a length of from 9 to 11 amino acids. The peptide binding site is formed solely by the α-chain and consists of a β-sheet consisting of eight strands, on which the peptides are clamped between two α-helices and fixed at both ends by hydrogen bridge bonds between the N- or C-termini of the bound peptide and the MHC complex.

The molecules of class II likewise describe a group of highly polymorphous membrane-located glycoproteins which have the function of antigen presentation. Unlike MHC class I molecules, however, these are normally expressed only in a group of special antigen-presenting cells (APC) and serve predominantly for the presentation of peptides of exogenously absorbed proteins. In humans, a distinction is made between three different types of class II molecules: HLA-DP, -DQ and -DR, of which a large number of different alleles in turn exist. The αβ-heterodimers formed consist of an α- and a β-chain, both of which are coded for in the MHC. Both chains possess a short cytoplasmic domain, a transmembrane domain and two extracellular domains (α1 and α2 or β1 and β2). The peptide binding groove is formed by the α1 and β1 domains of both chains (Brown, J. H., T. S. Jardetzky, J. C. Gorga, L. J. Stern, R. G. Urban, J. L. Strominger, and D. C. Wiley. 1993. Three-dimensional structure of the human class II histocompatibility antigen HLA-DR1. Nature 364:33-39). As with class I molecules, in this case the peptide is bound on a β-sheet between a plurality of α-helical structures. Unlike MHC class I molecules, however, the ends of the peptide binding site are open, so that the peptides are able to protrude from the complex (Rajnavolgyi, E., A. Horvath, P. Gogolak, G. K. Toth, G. Fazekas, M. Fridkin, and I. Pecht. 1997. Characterizing immunodominant and protective influenza hemagglutinin epitopes by functional activity and relative binding to major histocompatibility complex class II sites. Eur J Immunol 27:3105-3114). In vivo, the size of the bound peptides is normally in a range from 13 to 25 amino acids (AA) (Chicz, R. M., R. G. Urban, W. S. Lane, J. C. Gorga, L. J. Stern, D. A. Vignali, and J. L. Strominger. 1992. Predominant naturally processed peptides bound to HLA-DR1 are derived from MHC-related molecules and are heterogeneous in size. Nature 358:764-768, Rudensky, A., P. Preston-Hurlburt, S. C. Hong, A. Barlow, and C. A. Janeway, Jr. 1991. Sequence analysis of peptides bound to MHC class II molecules. Nature 353:622-627), but only 13 of them are fixed in the peptide binding fold of the MHC molecule. Crystallographic studies revealed that these 13 AAs are bound in an elongated conformation, similar to a polyproline type II helix. The AAs projecting from the peptide binding groove, on the other hand, can also assume other conformations (Jardetzky, T. S., J. H. Brown, J. C. Gorga, L. J. Stern, R. G. Urban, J. L. Strominger, and D. C. Wiley. 1996. Crystallographic analysis of endogenous peptides associated with HLA-DR1 suggests a common, polyproline II-like conformation for bound peptides. Proc Natl Acad Sci USA 93:734-738). More detailed studies of the peptide bond showed that 9 AAs of the peptide are responsible for the affinity of the bond (Hill, J. A., S. Southwood, A. Sette, A. M. Jevnikar, D. A. Bell, and E. Cairns. 2003. Cutting Edge: The Conversion of Arginine to Citrulline Allows for a High-Affinity Peptide Interaction with the Rheumatoid Arthritis-Associated HLA-DRB1*0401 MHC Class II Molecule. J Immunol 171:538-541). Lengthening of the peptides in either direction does not increase the affinity of the bond (Siklodi, B., A. B. Vogt, H. Kropshofer, F. Falcioni, M. Molina, D. R. Bolin, R. Campbell, G. J. Hammerling, and Z. A. Nagy. 1998. Binding affinity independent contribution of peptide length to the stability of peptide-HLA-DR complexes in live antigen presenting cells. Hum Immunol 59:463-471). Experiments with various alleles of the MHC class II molecule HLA-DR identified over the course of the binding fold a plurality of “binding pockets”, which are critical for the affinity of the peptide for the particular allele in question (O'Sullivan, D., T. Arrhenius, J. Sidney, M. F. Del Guercio, M. Albertson, M. Wall, C. Oseroff, S. Southwood, S. M. Colon, F. C. Gaeta, et al. 1991. On the interaction of promiscuous antigenic peptides with different DR alleles. Identification of common structural motifs. J Immunol 147:2663-2669; and Sette, A., J. Sidney, C. Oseroff, M. F. del Guercio, S. Southwood, T. Arrhenius, M. F. Powell, S. M. Colon, F. C. Gaeta, and H. M. Grey. 1993. HLA DR4w4-binding motifs illustrate the biochemical basis of degeneracy and specificity in peptide-DR interactions. J Immunol 151:3163-3170). For all allelic forms of HLA-DR, the binding in the first pocket close to the N-terminal end of the peptide appears to be decisive for the stability of the complex as a whole (Hammer, J., P. Valsasnini, K. Tolba, D. Bolin, J. Higelin, B. Takacs, and F. Sinigaglia. 1993. Promiscuous and allele-specific anchors in HLA-DR-binding peptides. Cell 74:197-203). However, the pockets at the relative positions P3, P4, P6, P7 and P9 as well as pockets at other positions of the binding fold appear, in an allele-specific manner, to be very important for the affinity of bound peptides (Southwood, S., J. Sidney, A. Kondo, M. F. del Guercio, E. Appella, S. Hoffman, R. T. Kubo, R. W. Chesnut, H. M. Grey, and A. Sette. 1998. Several common HLA-DR types share largely overlapping peptide binding repertoires. J Immunol 160:3363-3373). However, all alleles, even in the most stringent position, permit the binding of various amino acid side chains of the bound peptide, so that a large number of very different peptides can be bound to the same HLA allele (McFarland, B. J., and C. Beeson. 2002. Binding interactions between peptides and proteins of the class II major histocompatibility complex. Med Res Rev 22:168-203). The stability of the bond is determined, as well as by these allele-specific binding pockets, also by a number of hydrogen bridge bonds from highly conserved parts of the MHC molecule to the peptide backbone of the antigen. Unlike in MHC class I complexes, these are distributed over the entire peptide binding groove (Batalia, M. A., and E. J. Collins. 1997. Peptide binding by class I and class II MHC molecules. Biopolymers 43:281-302).

Starting from prior knowledge, changing the load state of MHC molecules with antigens appears to be a very promising approach to the therapy of pathological immune responses as well as of various pathologically excessive or absent immune responses. However, very different in vitro approaches for accelerating these processes have not hitherto yielded any practicable loading rates for MHC molecules or produced any findings which indicate corresponding usability of such MHC molecules.

For example, Jensen et al. (Jensen et al., J. Exp. Med., 1990, 171:1779-84) describe increasing the loading of MHC class II molecules by lowering the pH value. Jensen et al. (1990, supra) put forward the hypothesis that peptide replacement reactions might occur at the cell surface of activated APCs. Within the context of an infection, the density of presented, normally edited self-peptides, which otherwise are presented below the limiting value for activation of autoreactive T-cells, might be increased thereby. Jensen et al. (1990, supra) put forward local lowering of the pH value as a possible cause of such extracellular replacement reactions. However, local lowering of the pH value is possible substantially only in the case of MHC molecules that are soluble at such a pH, but not in the case of MHC molecules that are already present in insoluble form at such a pH. Furthermore, the loading of MHC molecules with antigens by lowering the pH value can be used only very briefly, or only after fixing of the cells, owing to the cell-damaging, non-physiological action. It is therefore not possible to use the process described according to Jensen et al. (1990, supra) for in vivo situations.

A further approach to the loading of MHC molecules with antigens takes place via the use of the natural catalyst for MHC ligand replacement, HLA-DM (Weber et al., J. Immunol., 2001, 167:5167-74). The method described by Weber et al. permits very efficient loading in principle, but the preparation of the HLA protein is very complex and expensive and accordingly is not obtainable commercially. Furthermore, the loading of MHC molecules with antigens in this method requires the presence of detergents and can only be carried out efficiently at a low pH value. Because the use of a low pH value and the use of detergents in Weber et al. (2001, supra) lead to non-physiological conditions, an in vivo use of the loaded MHC molecules obtained by this method is excluded.

A further alternative for the loading of MHC molecules with antigens is the use of destabilising detergents, which presumably are able to accelerate the loading of MHC class II molecules on account of their complex-destabilising influence (see Roof et al., Proc. Natl. Acad. Sci. U.S.A., 1990, 87:1735-9; Avva and Cresswell, Immunity, 1994, 1:763-74). However, destabilising detergents exhibit only a comparatively low activity in the loading of MHC molecules and have the obvious disadvantage that, after addition, it is not possible, or is possible only with extreme difficulty, to separate them from the MHC molecules. The separation of destabilising detergents and loaded MHC molecules therefore appears to be impossible under in vivo conditions, and MHC molecules loaded with the aid of destabilising detergents could therefore be introduced in vivo only together with the destabilising detergents used. However, because the use of such detergents leads to the decomposition or partial decomposition of cells, in vivo use of the loaded MHC molecules obtained by the method described by Roof et al. (Proc. Natl. Acad. Sci. U.S.A., 1990, 87:1735-9) or Avva and Cresswell (Immunity, 1994, 1:763-74) is not conceivable.

The best results obtained hitherto when loading MHC molecules with antigens were achieved by the use of low molecular weight compounds, such as so-called small molecules such as, for example, para-chlorophenol, which act as H donors (see Falk et al., J. Biol. Chem., 2002, 277:2709-15; and Marin-Esteban et al., J. Autoimmun., 2003, 20:63-9; and WO 03/016512). In these studies it has been shown that so-called small molecules with H-donor properties possess the ability to catalyse the loading of MHC class I and class II molecules with peptides at physiological pH value or to replace bound antigens at the surface of APCs at physiological pH value. However, all the substances identified in these studies that are capable of influencing MHC class II ligand interactions can be used only at relatively high, non-physiological concentrations. For example, the use of phenol requires a concentration of 30 mM, n-propanol even a concentration of over 200 mM, which concentrations already have a considerable toxic action in vivo. In order to develop their activity, concentrations that are toxic in vivo would have to be used, so that it is neither possible nor conceivable to test their activity in animal models and accordingly their potential association with various pathologically excessive or absent immune responses.

The problem of catalysis for the efficient loading of MHC molecules with ligands using minimal concentrations of catalyst compounds has not yet been solved in the art.

The object underlying the present invention is, therefore, to provide a method for changing the load state of MHC molecules with ligands, which method permits an efficient loading of the MHC molecules with ligands, the replacement of ligands on the surface of MHC molecules, the decrease or removal of ligands on the surface of MHC molecules, and the subsequent in vivo use thereof.

A further object of the present invention is to provide therapeutic agents or diagnostic agents, or methods which can be carried out with such therapeutic agents or diagnostic agents, for the treatment or detection of disorders or conditions that are associated with various pathologically excessive or absent immune responses.

According to the invention, the object underlying the invention is achieved by a method for changing the load state of MHC molecules with ligands. This method comprises the following steps:
a) providing a composition containing MHC molecules; and
b) adding a catalyst selected from a compound of formulae I or IA having the following structure: 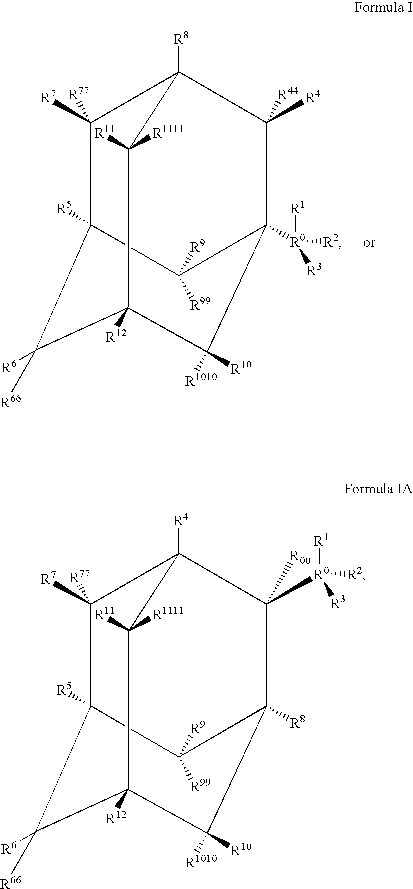
   wherein:
   R⁰, R⁰⁰, R¹, R², R³, R⁴, R⁵, R⁶, R⁷, R⁸, R⁹, R¹⁰, R¹¹, R¹², R⁴⁴, R⁶⁶, R⁷⁷, R⁹⁹, R¹⁰¹⁰ and R¹¹¹¹ can be a bond or are selected independently of one another from a group consisting of:
      H, O, S, N,
      OH, OR¹³,
      SH, SO, SO₂, SO₂R¹³, SO₃, HSO₃, SR¹³, SR¹³R¹⁴, S(CH₂)ₙR¹³, S(CHₙ)R¹³; S(CH₂)ₙ(CH)ₙR¹³, S(CH₂)ₙ(CH)ₙR¹³,
      NH, NH₂, NHNH₂, NHR¹³, NR¹³R¹⁴, NO, NO₂, NOH, NOR¹³,
      X, CX₃, CHX₂, CH₂X, CR¹³X₂, CR₂¹³X, CR₃¹³, wherein X=halogen,
      CN, CO, COR¹³, COOH, COOR¹³,
      CH₃, (CH₂)ₙCH₃; (CH)ₙCH₃; (CH₂)ₙR¹³, (CH)ₙR¹³, (CH)ₙ(CH₂)ₙCH₃; (CH₂)ₙ(CH)ₙCH₃; (CH)ₙ(CH₂)ₙR¹³; (CH₂)ₙ(CH)ₙR¹³; C(R¹³)C(R¹⁴)CH₃, C(R¹³)(CH₂)ₙR¹⁴, (CH₂)ₙR¹³, (CH)ₙ(OH)R¹³; (CH₂)ₙ(OH)R¹³; (CH)ₙ(OH)CH₃; (CH₂)ₙ(OH)CH₃; OCH₃, O(CH₂)ₙCH₃, O(CH)ₙCH₃, O(CH₂)ₙR¹³, O(CH)ₙR¹³, O(CH)ₙ(CH₂)ₙR¹³, O(CH₂)ₙ(CH)ₙR¹³, (CH₂)ₙOCH₃, (CH)ₙOCH₃, (CH₂)ₙOR¹³, (CH)ₙOR¹³, (CH)ₙ(CH₂)ₙOR¹³, (CH₂)ₙ(CH)ₙOR¹³, (CH)ₙ(OH)CH₃; (CH₂)ₙ(OH)CH₃; (CH)ₙ(OH)R¹³; (CH₂)ₙ(OH)R¹³; (CH₂)ₙCH₂X; (CH)ₙCH₂X; (CH₂)ₙCH₂X; (CH₂)ₙX, (CH)ₙX, (CH)ₙ(CH₂)ₙCH₂X; (CH₂)ₙ(CH)ₙCH₂X; (CH)ₙ(CH₂)ₙX; (CH₂)ₙ(CH)ₙX; OCH₂X, O(CH₂)ₙCH₂X, O(CH)ₙCH₂X, O(CH₂)ₙX, O(CH)ₙX, O(CH)ₙ(CH₂)ₙX, O(CH₂)ₙ(CH)ₙX, (CH₂)ₙOCH₃, (CH)ₙOCH₂X, (CH₂)ₙNHR¹³, (CH₂)ₙNHOR¹³, (CH₂)ₙNHCOR¹³, (CH₂)ₙN(R¹³)CO, N(R¹³)(CH₂)ₙR¹⁴, N(R¹³)(CH)ₙR¹⁴, N(R¹³)(CH)ₙ(CH₂)ₙR¹⁴, N(R¹³)(CH₂)ₙ(CH)ₙR¹⁴, N(R¹³)COR¹⁴, N(R¹³)COOR¹⁴, CONH₂, CONHCH₃, C₃H₆OH, C(NH₂)(CH₂)ₙ(OH), OCONH(CH₂)ₙCH₃; OCONH(CH)ₙCH₃; OCONH(CH)ₙ(CH₂)ₙCH₃; OCONH(CH₂)ₙ(CH)ₙCH₃; (CH)ₙOR¹³, (CH₂)ₙOR¹³, C₆N₂H₅, C₆H₄(NHCOCH₃), C₆H₄SO₂NH, (CNNHC(CONHNH₂)CH₂), and C₆N₂H₇,
      wherein n=from 1 to 30, and
      R¹³ and R¹⁴ are selected independently of one another from a group consisting of
      H, O, S, N,
      OH, OR¹⁵,
      SH, SO, SO₂, SO₃, HSO₃, SR¹⁵, SR¹⁵R¹⁶, S(CH₂)ₙR¹⁵, S(CHₙ)R¹⁵; S(CH₂)ₙ(CH)ₙR¹⁵, S(CH₂)ₙ(CH)ₙR¹⁵,
      NH, NH₂, NHNH₂, NHR¹⁵, NR¹⁵R¹⁶, NO, NO₂, NOH, NOR¹⁵,
      X, CX₃, CHX₂, CH₂X, CR¹⁵X₂, CR₂¹⁵X, CR₃¹⁵, wherein X=halogen,
      CN, CO, COR¹⁵, COOH, COR¹⁵, COOR¹⁵,
      CH₃, (CH₂)ₙCH₃; (CH)ₙCH₃; (CH₂)ₙR¹⁵, (CH)ₙR¹⁵, (CH)ₙ(CH₂)ₙCH₃; (CH₂)ₙ(CH)ₙCH₃; (CH)ₙ(CH₂)ₙR¹⁵; (CH₂)ₙ(CH)ₙR¹⁵; OCH₃, O(CH₂)ₙCH₃, O(CH)ₙCH₃, O(CH₂)ₙR¹⁵, O(CH)ₙR¹⁵, O(CH)ₙ(CH₂)ₙR¹⁵, O(CH₂)ₙ(CH)ₙR¹⁵, (CH₂)ₙOCH₃, (CH)ₙOCH₃, (CH₂)ₙOR¹⁵, (CH)ₙOR¹⁵, (CH)ₙ(CH₂)ₙOR¹⁵, (CH₂)ₙ(CH)ₙOR¹⁵, (CH)ₙ(OH)CH₃; (CH₂)ₙ(OH)CH₃; (CH)ₙ(OH)R¹⁵; (CH₂)ₙ(OH)R¹⁵; (CH₂)ₙCH₂X; (CH)ₙCH₂X; (CH₂)ₙCH₂X; (CH₂)ₙX, (CH)ₙX, (CH)ₙ(CH₂)ₙCH₂X; (CH₂)ₙ(CH)ₙCH₂X; (CH)ₙ(CH₂)ₙX; (CH₂)ₙ(CH)ₙX; OCH₂X, O(CH₂)ₙCH₂X, O(CH)ₙCH₂X, O(CH₂)ₙX, O(CH)ₙX, O(CH)ₙ(CH₂)ₙX, O(CH₂)ₙ(CH)ₙX, (CH₂)ₙOCH₃, (CH)ₙOCH₂X, (CH₂)ₙNHR¹⁵, (CH₂)ₙNHOR¹⁵, (CH₂)ₙNHCOR¹⁵, NR¹⁵(CH₂)ₙR¹⁶, NR¹⁵(CH)ₙR¹⁶, NR¹⁵(CH)ₙ(CH₂)ₙR¹⁶, NR¹⁵(CH₂)ₙ(CH)ₙR¹⁶, OCONH(CH₂)ₙCH₃; OCONH(CH)ₙCH₃; OCONH(CH)ₙ(CH₂)ₙCH₃; OCONH(CH₂)ₙ(CH)ₙCH₃; (CH)ₙOR¹⁵, (CH₂)ₙOR¹³, C₆N₂H₅, C₆H₄(NHCOCH₃), C₆H₄SO₂NH, (CNNHC(CONHNH₂)CH₂), adamantane, triazole, tetrazole, pyrazole, and oxazole;
      wherein n=from 1 to 30, and
      R¹⁵ and R¹⁶ are selected independently of one another from a group consisting of
      H, O, S, N,
      OH,
      SH, SO, SO₂, SO₃, HSO₃,
      NH, NH₂, NHNH₂, NO, NO₂, NHNH₂, NOH,
      X, CX₃, CHX₂, CH₂X, wherein X=halogen,
      CN, CO, COOH,
      CH₃, (CH₂)ₙCH₃; (CH)ₙCH₃; (CH)ₙ(CH₂)ₙCH₃; (CH₂)ₙ(CH)ₙCH₃; OCH₃, O(CH₂)ₙCH₃, O(CH)ₙCH₃, (CH₂)ₙOCH₃, (CH)ₙOCH₃, (CH)ₙ(OH)CH₃; (CH₂)ₙ(OH)CH₃; (CH₂)ₙCH₂X; (CH)ₙCH₂X; (CH₂)ₙCH₂X; (CH₂)ₙX, (CH)ₙX, (CH)ₙ(CH₂)ₙCH₂X; (CH₂)ₙ(CH)ₙCH₂X; (CH)ₙ(CH₂)ₙX; (CH₂)ₙ(CH)ₙX; OCH₂X, O(CH₂)ₙCH₂X, O(CH)ₙCH₂X, O(CH₂)ₙX, O(CH)ₙX, O(CH)ₙ(CH₂)ₙX, O(CH₂)ₙ(CH)ₙX, (CH₂)ₙOCH₃, (CH)ₙOCH₂X, OCONH(CH₂)ₙCH₃; OCONH(CH)ₙCH₃; OCONH(CH)ₙ(CH₂)ₙCH₃; OCONH(CH₂)ₙ(CH)ₙCH₃; C₆N₂H₅, C₆H₄(NHCOCH₃), C₆H₄SO₂NH, (CNNHC(CONHNH₂)CH₂), adamantane, triazole, tetrazole, pyrazole, and oxazole;
      and/or
      R⁰, R⁰⁰, R¹, R², R³, R⁴, R⁵, R⁶, R⁷, R⁸, R⁹, R¹⁰, R¹¹, R¹², R⁴⁴, R⁶⁶, R⁷⁷, R⁹⁹, R¹⁰¹⁰ and R¹¹¹¹ are selected independently of one another from a group consisting of a branched or unbranched C₁-C₃₀-alkyl, C₁-C₃₀-alkenyl, C₁-C₃₀-heteroalkyl, C₁-C₃₀-heteroalkenyl, C₁-C₃₀-alkoxy, C₁-C₃₀-alkenoxy, C₁-C₃₀, C₃-C₈-cycloalkyl, C₃-C₈-cycloalkenyl, C₅-C₃₀-aryl, C₅-C₃₀-heteroaryl, arylalkyl, arylalkenyl, C₅₋₂₀-aryloxy, heteroarylalkyl, heteroarylalkenyl, heterocycloalkyl, heterocycloalkenyl, carboxamido, acylamino, amidino, heteroaryloxy residue, adamantane, triazole, tetrazole, pyrazole, toluene, aniline, benzaldehyde, anisole, benzonitrile, phenol, acetophenone, benzoic acid, xylene, styrene, naphthalene, anthracene, phenanthrene, naphthalene, anthracene, phenanthrene, benzpyrene, pyridine, pyrimidine, purine, pyrrolidine, tetrahydrofuran, tetrahydrothiophene, tetrahydropyran, piperidine, pyrrole, furan, thiophene, pyridine, quinoline, indole, pyrimidine, pyrazine, purine, imidazole, pteridine, acridine, chromane, chromene, coumarin (chromen-2-one), and oxazole,
c) changing the load state of the MHC molecules; and
d) isolating the MHC molecules whose load state has been changed.

In a particularly preferred embodiment of the present invention, the compounds of formulae I or IA are defined as follows:
R⁰, R⁰⁰, R¹, R², R³, R⁴, R⁵, R⁶, R⁷, R⁸, R⁹, R¹⁰, R¹¹, R¹², R⁴⁴, R⁶⁶, R⁷⁷, R⁹⁹, R¹⁰¹⁰ and R¹¹¹¹ can be selected together or independently of one another from a group consisting of:
   H, O, S, N,
   OH, OR¹³,
   SH, SO, SO₂, SO₂R¹³, SO₃, HSO₃, SR¹³, SR¹³R¹⁴, S(CH₂)ₙR¹³, S(CHₙ)R¹³; S(CH₂)ₙ(CH)ₙR¹³, S(CH₂)ₙ(CH)ₙR¹³,
   NH, NH₂, NH₂, NHR¹³, NR¹³R¹⁴, NO, NO₂, NOH, NOR¹³,
   X, CX₃, CHX₂, CH₂X, CR¹³X₂, CR₂¹³X, CR₃¹³, wherein X=halogen,
   CN, CO, COR¹³, COOH, COOR¹³,
   CH₃, (CH₂)ₙCH₃; (CH)ₙCH₃; (CH₂)ₙR¹³, (CH)ₙR¹³, (CH)ₙ(CH₂)ₙCH₃; (CH₂)ₙ(CH)ₙCH₃; (CH)ₙ(CH₂)ₙR¹³; (CH₂)ₙ(CH)ₙR¹³; C(R¹³)C(R¹⁴)CH₃, C(R¹³)(CH₂)ₙR¹⁴, (CH₂)ₙR¹³, (CH)ₙ(OH)R¹³; (CH₂)ₙ(OH)R¹³; (CH)ₙ(OH)CH₃; (CH₂)ₙ(OH)CH₃; OCH₃, O(CH₂)ₙCH₃, O(CH)ₙCH₃, O(CH₂)ₙR¹³, O(CH)ₙR¹³, O(CH)ₙ(CH₂)ₙR¹³, O(CH₂)ₙ(CH)ₙR¹³, (CH₂)ₙOCH₃, (CH)ₙOCH₃, (CH₂)ₙOR¹³, (CH)ₙOR¹³, (CH)ₙ(CH₂)ₙOR¹³, (CH₂)ₙ(CH)ₙOR¹³, (CH)ₙ(OH)CH₃; (CH₂)ₙ(OH)CH₃; (CH)ₙ(OH)R¹³; (CH₂)ₙ(OH)R¹³; (CH₂)ₙCH₂X; (CH)ₙCH₂X; (CH₂)ₙCH₂X; (CH₂)ₙX, (CH)ₙX, (CH)ₙ(CH₂)ₙCH₂X; (CH₂)ₙ(CH)ₙCH₂X; (CH)ₙ(CH₂)ₙX; (CH₂)ₙ(CH)ₙX; OCH₂X, O(CH₂)ₙCH₂X, O(CH)ₙCH₂X, O(CH₂)ₙX, O(CH)ₙX, O(CH)ₙ(CH₂)ₙX, O(CH₂)ₙ(CH)ₙX, (CH₂)ₙOCH₃, (CH)ₙOCH₂X, (CH₂)ₙNHR¹³, (CH₂)ₙNHOR¹³, (CH₂)ₙNHCOR¹³, (CH₂)ₙN(R¹³)CO, N(R¹³)(CH₂)ₙR¹⁴, N(R¹³)(CH)ₙR¹⁴, N(R¹³)(CH)ₙ(CH₂)ₙR¹⁴, N(R¹³)(CH₂)ₙ(CH)ₙR¹⁴, N(R¹³)COR¹⁴, N(R¹³)COOR¹⁴, CONH₂, CONHCH₃, C₃H₆OH, C(NH₂)(CH₂)ₙ(OH), OCONH(CH₂)ₙCH₃; OCONH(CH)ₙCH₃; OCONH(CH)ₙ(CH₂)ₙCH₃; OCONH(CH₂)ₙ(CH)ₙCH₃; (CH)ₙOR¹³, (CH₂)ₙOR¹³, C₆N₂H₅, C₆H₄(NHCOCH₃), C₆H₄SO₂NH, and (CNNHC(CONHNH₂)CH₂), C₆N₂H₇,
   wherein n=from 1 to 10, and
   R¹³ and R¹⁴ are selected independently of one another from a group consisting of
   H, O, S, N,
   OH, OR¹⁵,
   SH, SO, SO₂, SO₃, HSO₃, SR¹⁵, SR¹⁵R¹⁶, S(CH₂)ₙR¹⁵, S(CHₙ)R¹⁵; S(CH₂)ₙ(CH)ₙR¹⁵, S(CH₂)ₙ(CH)ₙR¹⁵,
   NH, NH₂, NHNH₂, NHR¹⁵, NR¹⁵R¹⁶, NO, NO₂, NOH, NOR¹⁵,
   X, CX₃, CHX₂, CH₂X, CR¹⁵X₂, CR₂¹⁵X, CR₃¹⁵, wherein X=halogen,
   CN, CO, COR¹⁵, COOH, COR¹⁵, COOR¹⁵,
   CH₃, (CH₂)ₙCH₃; (CH)ₙCH₃; (CH₂)ₙR¹⁵, (CH)ₙR¹⁵, (CH)ₙ(CH₂)ₙCH₃; (CH₂)ₙ(CH)ₙCH₃; (CH)ₙ(CH₂)ₙR¹⁵; (CH₂)ₙ(CH)ₙR¹⁵; OCH₃, O(CH₂)ₙCH₃, O(CH)ₙCH₃, O(CH₂)ₙR¹⁵, O(CH)ₙR¹⁵, O(CH)ₙ(CH₂)ₙR¹⁵, O(CH₂)ₙ(CH)ₙR¹⁵, (CH₂)ₙOCH₃, (CH)ₙOCH₃, (CH₂)ₙOR¹⁵, (CH)ₙOR¹⁵, (CH)ₙ(CH₂)ₙOR¹⁵, (CH₂)ₙ(CH)ₙOR¹⁵, (CH)ₙ(OH)CH₃; (CH₂)ₙ(OH)CH₃; (CH)ₙ(OH)R¹⁵; (CH₂)ₙ(OH)R¹⁵; (CH₂)ₙCH₂X; (CH)ₙCH₂X; (CH₂)ₙCH₂X; (CH₂)ₙX, (CH)ₙX, (CH)ₙ(CH₂)ₙCH₂X; (CH₂)ₙ(CH)ₙCH₂X; (CH)ₙ(CH₂)ₙX; (CH₂)ₙ(CH)ₙX; OCH₂X, O(CH₂)ₙCH₂X, O(CH)ₙCH₂X, O(CH₂)ₙX, O(CH)ₙX, O(CH)ₙ(CH₂)ₙX, O(CH₂)ₙ(CH)ₙX, (CH₂)ₙOCH₃, (CH)ₙOCH₂X, (CH₂)ₙNHR¹⁵, (CH₂)ₙNHOR¹⁵, (CH₂)ₙNHCOR¹⁵, NR¹⁵(CH₂)ₙR¹⁶, NR¹⁵(CH)ₙR¹⁶, NR¹⁵(CH)ₙ(CH₂)ₙR¹⁶, NR¹⁵(CH₂)ₙ(CH)ₙR¹⁶, OCONH(CH₂)ₙCH₃; OCONH(CH)ₙCH₃; OCONH(CH)ₙ(CH₂)ₙCH₃; OCONH(CH₂)ₙ(CH)ₙCH₃; (CH)ₙOR¹⁵, (CH₂)ₙOR¹³, C₆N₂H₅, C₆H₄(NHCOCH₃), C₆H₄SO₂NH, (CNNHC(CONHNH₂)CH₂), adamantane, triazole, tetrazole, pyrazole, and oxazole;
   wherein n=from 1 to 10, and
   R¹⁵ and R¹⁶ are selected independently of one another from a group consisting of
   H, O, S, N,
   OH,
   SH, SO, SO₂, SO₃, HSO₃,
   NH, NH₂, NHNH₂, NO, NO₂, NHNH₂, NOH,
   X, CX₃, CHX₂, CH₂X, wherein X=halogen,
   CN, CO, COOH,
   CH₃, (CH₂)ₙCH₃; (CH)ₙCH₃; (CH)ₙ(CH₂)ₙCH₃; (CH₂)ₙ(CH)ₙCH₃; OCH₃, O(CH₂)ₙCH₃, O(CH)ₙCH₃, (CH₂)ₙOCH₃, (CH)ₙOCH₃, (CH)ₙ(OH)CH₃; (CH₂)ₙ(OH)CH₃; (CH₂)ₙCH₂X; (CH)ₙCH₂X; (CH₂)ₙCH₂X; (CH₂)ₙX, (CH)ₙX, (CH)ₙ(CH₂)ₙCH₂X; (CH₂)ₙ(CH)ₙCH₂X; (CH)ₙ(CH₂)ₙX; (CH₂)ₙ(CH)ₙX; OCH₂X, O(CH₂)ₙCH₂X, O(CH)ₙCH₂X, O(CH₂)ₙX, O(CH)ₙX, O(CH)ₙ(CH₂)ₙX, O(CH₂)ₙ(CH)ₙX, (CH₂)ₙOCH₃, (CH)ₙOCH₂X, OCONH(CH₂)ₙCH₃; OCONH(CH)ₙCH₃; OCONH(CH)ₙ(CH₂)ₙCH₃; OCONH(CH₂)ₙ(CH)ₙCH₃; C₆N₂H₅, C₆H₄(NHCOCH₃), C₆H₄SO₂NH, (CNNHC(CONHNH₂)CH₂), adamantane, triazole, tetrazole, pyrazole, and oxazole.

In a more greatly preferred embodiment of the present invention, the compounds of formula I or IA are defined by the following structures I 1 to I 36:

TABLE 1
Catalysts according to the invention of formulae I and IA

| | |
|---|---|
| | I 1 |
| 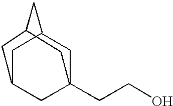 | |
| | I 2 |
| 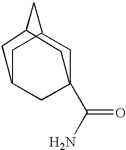 | |
| | I 3 |
| 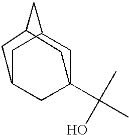 | |
| | I 4 |
| 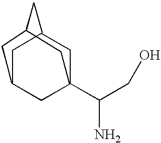 | |
| | I 5 |
| 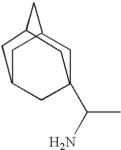 | |
| | I 6 |
| 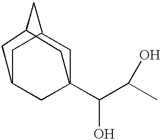 | |
| | I 7 |
| 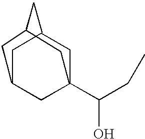 | |
| | I 8 |
| 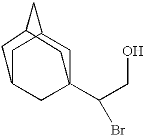 | |
| | I 9 |
| 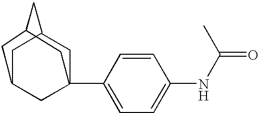 | |

| | |
|---|---|
| | I 10 |
| 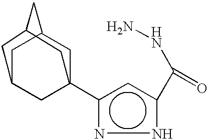 | |
| | I 11 |
| 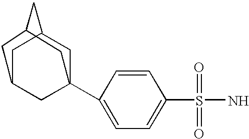 | |
| | I 12 |
| 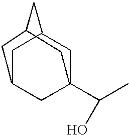 | |
| | I 13 |
| 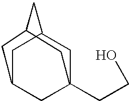 | |
| | I 14 |
| 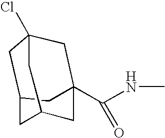 | |
| | I 15 |
| 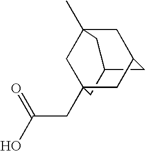 | |
| | I 16 |
| 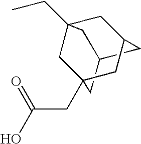 | |
| | I 17 |
| 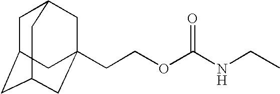 | |
| | I 18 |
| 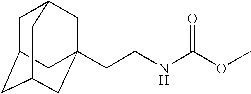 | |
| | I 19 |
| 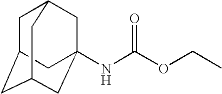 | |
| | I 20 |
| 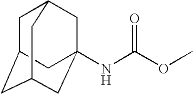 | |
| | I 21 |
| 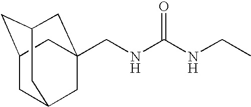 | |
| | I 22 |
| 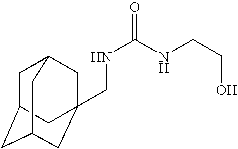 | |
| | I 23 |
| 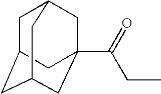 | |
| | I 24 |
| 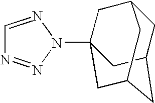 | |
| | I 25 |
| 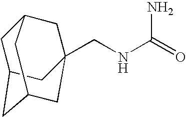 | |
| | I 26 |
| 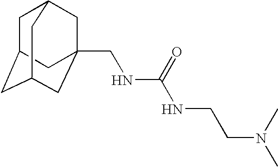 | |
| | I 27 |
| 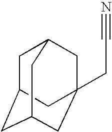 | |
| | I 28 |
| 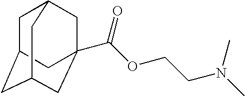 | |
| | I 29 |
| 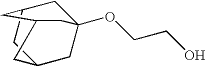 | |
| | I 30 |
| 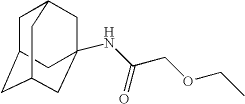 | |
| | I 31 |
| 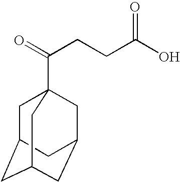 | |
| | I 32 |
| 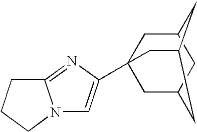 | |
| | I 33 |
| 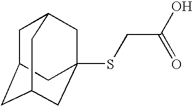 | |
| | I 34 |
| 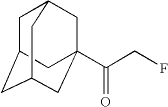 | |
| | I 35 |
| 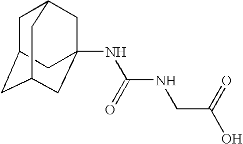 | |
| | I 36 |
| 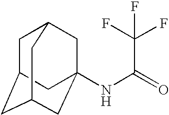 | |

The compounds that come under formulae I and IA are also referred to generically in the present invention as adamantyl compounds, because they are based on a preferably substituted adamantane basic structure. In principle, all substituted adamantane basic structures can be used for carrying out any method disclosed herein. All these conceivable substituted adamantane basic structures are adamantyl compounds. The spherosymmetrical adamantane basic structure can be substituted one, two, three or more times, optionally also on all the carbon atoms (10 in total) that form the adamantane basic structure. The adamantane basic structure contains different types of carbon atoms, namely carbons coordinated twice or three times in the matrix. While only one substituent can occur in each case at the triply coordinated carbon atoms, there can be two substituents in each case at the doubly coordinated carbon atoms. It is preferred, however, to introduce only one substituent at the carbons doubly coordinated in the matrix. Although it is accordingly possible for a total of 16 substitutions to be provided in the basic adamantane structure, in order to provide compounds for methods according to the invention, for example for load changing or for diagnosis, preference is given according to the invention to those substituted adamantyl structures that have one, two or three substituents, preferably at different carbon atoms. Particular preference is given to those compounds that have a long-chained substituent at one carbon atom in the matrix, whether it be a doubly or triply coordinated carbon atom. A long-chained substituent within the scope of this invention is typically distinguished by at least 8 atoms bonded together via covalent bonds to form a chain, for example a C8-alkyl or C8-alkoxy, or alternatively 8 atoms bonded together via amide-like linkages. If further substituents occur, preferably on at least one further carbon atom in the adamantane matrix, these substituents are preferably short-chained and typically have 6 or fewer carbon atoms bonded together to form a chain. Particular preference is given to monosubstituted adamantyl compounds with one substituent having a chain length of at least 8 atoms.

In an alternative embodiment of the present invention, the underlying object is achieved by a method for changing the load state of MHC molecules with ligands that comprises the following steps:
a) providing a composition containing MHC molecules; and
b) adding a catalyst selected from a compound of formula II having the following structure: 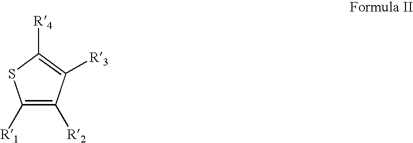
   wherein:
   R^{1′}, R^{2′}, R^{3′} and R^{4′} can be a bond or are selected independently of one another from a group consisting of:
      H, O, S, N,
      OH, OR^{13′}, SH, SO, SO₂, SO₂R^{13′}, SO₃, HSO₃, SR^{13′}, SR^{13′}R^{14′},
      X, CX₃, CHX₂, CH₂X, CR^{13′}X₂, CR₂^{13′}X, CR₃^{13′} wherein X=halogen,
      CN, CO, COOH, COOR^{13′},
      NH, NH₂, NHR^{13′}, NR^{13′}R^{14′}, NO, NO₂, NOH, NOR^{13′},
      CH₃, (CH₂)ₙCH₃, (CH)ₙCH₃, (CH₂)ₙR^{13′}, (CH)R^{13′}, OCH₃, O(CH₂)ₙ, O(CH₂)ₙCH₃, O(CH₂)R^{13′}, (CH₂)ₙOH, (CH)ₙOH, (CH₂)ₙ(CH)ₙCH₃, (CH)ₙ(CH₂)ₙCH₃, (CH₂)ₙ(CH)ₙR^{13′}, (CH)ₙ(CH₂)ₙR^{13′}, —(C₃HNO)—CHX₂, (C₃HNO)—COOR^{13′}, —(C₃HNO)—CHR^{13′}R^{14′}, wherein n=from 1 to 30, and
      R^{13′} and R^{14′} are selected independently of one another from a group consisting of
      H, O, S, N,
      OH, OR^{15′}, SH, SO, SO₂, SO₃, HSO₃, SR^{15′}, SR^{15′}R^{16′},
      X, CX₃, CHX₂, CH₂X, CR^{15′}X₂, CR₂^{15′}X, CR₃^{15′} wherein X=halogen,
      CN, CO, COOH, COOR^{15′},
      NH, NH₂, NHR^{15′}, NR^{15′}R^{16′}, NO, NO₂, NOH, NOR^{15′},
      CH₃, (CH₂)ₙCH₃, (CH)ₙCH₃, (CH₂)ₙR^{15′}, (CH)ₙR^{15′}, OCH₃, O(CH₂)ₙ, O(CH₂)ₙCH₃, O(CH₂)ₙR^{15′}, (CH₂)ₙOH, (CH)ₙOH, (CH₂)ₙ(CH)ₙCH₃, (CH)ₙ(CH₂)ₙCH₃, (CH₂)ₙ(CH)R^{15′}, (CH)ₙ(CH₂)ₙR^{15′}, —(C₃HNO)—CHX₂, —(C₃HNO)—CHR^{15′}R^{15′},
      wherein n=from 1 to 30,
      R^{15′} and R^{16′} are selected independently of one another from a group consisting of
      H, O, S, N,
      OH, SH, SO, SO₂, SO₃, HSO₃,
      X, CX₃, CHX₂, CH₂X, wherein X=halogen,
      CN, CO, COOH,
      NH, NH₂, NO, NO₂, NOH,
      CH₃, (CH₂)ₙCH₃, (CH)ₙCH₃, OCH₃, O(CH₂)ₙ, O(CH₂)ₙCH₃, (CH₂)ₙOH, (CH)ₙOH, (CH₂)ₙ(CH)ₙCH₃, (CH)ₙ(CH₂)ₙCH₃, —(C₃HNO)—CHX₂, wherein n=from 1 to 30,
      and/or
      R^{1′}, R^{2′}, R^{3′} and R^{4′} are selected independently of one another from a group consisting of a branched or unbranched C₁-C₃₀-alkyl, C₁-C₃₀-alkenyl, C₁-C₃₀-heteroalkyl, C₁-C₃₀-heteroalkenyl, C₁-C₃₀-alkoxy, C₁-C₃₀-alkenoxy, C₁-C₃₀-acyl, C₃-C₈-cycloalkyl, C₃-C₈-cycloalkenyl, C₅-C₃₀-aryl, C₅-C₃₀-heteroaryl, arylalkyl, arylalkenyl, C₅₋₃₀-aryloxy, heteroarylalkyl, heteroarylalkenyl, heterocycloalkyl, heterocycloalkenyl, carboxamido, acylamino, amidino, adamantyl residue, heteroaryloxy residue, toluene, aniline, benzaldehyde, anisole, benzonitrile, phenol, acetophenone, benzoic acid, xylene, styrene, naphthalene, anthracene, phenanthrene, naphthalene, anthracene, phenanthrene, benzpyrene, pyridine, pyrimidine, purine, pyrrolidine, tetrahydrofuran, tetrahydrothiophene, tetrahydropyran, piperidine, pyrrole, furan, thiophene, pyridine, quinoline, indole, pyrimidine, pyrazine, purine, imidazole, pteridine, acridine, chromane, chromene, and coumarin (chromen-2-one), adamantane, pyrazole, diazole, tetrazole, triazole, and/or
   R^{1′} and R^{2′} together can form a bridged structure selected from a branched or unbranched C₁-C₈-alkyl, C₁-C₈-alkenyl, C₁-C₈-heteroalkyl, C₁-C₈-heteroalkenyl, C₁-C₈-alkoxy, C₁-C₈-alkenoxy, C₁-C₈-acyl, C₃-C₈-cycloalkyl, C₃-C₈-cycloalkenyl, C₅-C₈-aryl, C₅-C₈-heteroaryl, arylalkyl, arylalkenyl, C₅₋₈-aryloxy, heteroarylalkyl, heteroarylalkenyl, heterocycloalkyl, heterocycloalkenyl, carboxamido, acylamino, amidino, adamantyl residue, heteroaryloxy residue, toluene, aniline, benzaldehyde, anisole, benzonitrile, phenol, acetophenone, benzoic acid, xylene, styrene, naphthalene, anthracene, phenanthrene, naphthalene, anthracene, phenanthrene, benzpyrene, pyridine, pyrimidine, purine, pyrrolidine, tetrahydrofuran, tetrahydrothiophene, tetrahydropyran, piperidine, pyrrole, furan, thiophene, pyridine, quinoline, indole, pyrimidine, pyrazine, purine, imidazole, pteridine, acridine, chromane, chromene, and coumarin (chromen-2-one), adamantane, pyrazole, diazole, tetrazole, triazole, wherein one or two substituents selected from R^{1′} and R^{2′} (as described individually hereinbefore) can occur independently of one another at each individual atom of the bridged structure, preferably from 1 to 12 atoms,
c) changing the load state of the MHC molecules; and
d) isolating the MHC molecules whose load state has been changed.

In a particularly preferred embodiment, the substituents of formula II are defined as follows.
R^{1′}, R^{2′}, R^{3′} or R^{4′} can be a bond or can be selected together or independently of one another from a group consisting of:
   H, O, S, N,
   OH, OR^{13′}, SH, SO, SO₂, SO₂R^{13′}, SO₃, HSO₃, SR^{13′}, SR^{13′}R^{14′},
   X, CX₃, CHX₂, CH₂X, CR^{13′}X₂, CR₂^{13′}X, CR₃^{13′} wherein X=halogen,
   CN, CO, COOH, COOR^{13′},
   NH, NH₂, NHR^{13′}, NR^{13′}R^{14′}, NO, NO₂, NOH, NOR^{13′}, CH₃, (CH₂)ₙCH₃, (CH)ₙCH₃, (CH₂)ₙR^{13′}, (CH)ₙR^{13′}, OCH₃, O(CH₂)ₙ, O(CH₂)ₙCH₃, O(CH₂)ₙR^{13′}, (CH₂)ₙOH, (CH)ₙOH, (CH₂)ₙ(CH)ₙCH₃, (CH)ₙ(CH₂)ₙCH₃, (CH₂)ₙ(CH)ₙR^{13′}, (CH)ₙ(CH₂)ₙR^{13′}, —(C₃HNO)—CHX₂, (C₃HNO)—COOR^{13′}, —(C₃HNO)—CHR^{13′}R^{14′}, wherein n=from 1 to 10, and
   R^{13′} and R^{14′} are selected independently of one another from a group consisting of
   H, O, S, N,
   OH, OR^{15′}, SH, SO, SO₂, SO₃, HSO₃, SR^{15′}, SR^{15′}R^{16′},
   X, CX₃, CHX₂, CH₂X, CR^{15′}X₂, CR₂^{15′}X, CR₃^{15′} wherein X=halogen,
   CN, CO, COOH, COOR^{15′},
   NH, NH₂, NHR^{15′}, NR^{15′}R^{16′}, NO, NO₂, NOH, NOR^{15′},
   CH₃, (CH₂)ₙCH₃, (CH)ₙCH₃, (CH₂)ₙR^{15′}, (CH)ₙR^{15′}, OCH₃, O(CH₂)ₙ, O(CH₂)ₙCH₃, O(CH₂)ₙR^{15′}, (CH₂)ₙOH, (CH)ₙOH, (CH₂)ₙ(CH)ₙCH₃, (CH)ₙ(CH₂)ₙCH₃, (CH₂)ₙ(CH)ₙR^{15′}, (CH)ₙ(CH₂)ₙR^{15′}, —(C₃HNO)—CHX₂, —(C₃HNO)—CHR^{15′}R^{16′},
   wherein n=from 1 to 10, and
   R^{15′} and R^{16′} are selected independently of one another from a group consisting of
   H, O, S, N,
   OH, SH, SO, SO₂, SO₃, HSO₃,
   X, CX₃, CHX₂, CH₂X, wherein X=halogen,
   CN, CO, COOH,
   NH, NH₂, NO, NO₂, NOH,
   CH₃, (CH₂)ₙCH₃, (CH)ₙCH₃, OCH₃, O(CH₂)ₙ, O(CH₂)ₙCH₃, (CH₂)ₙOH, (CH)ₙOH, (CH₂)ₙ(CH)ₙCH₃, (CH)ₙ(CH₂)ₙCH₃, —(C₃HNO)—CHX₂, wherein n=from 1 to 10,
   and/or
R^{1′} and R^{2′} together can form a bridged structure selected from a branched or unbranched C₁-C₈-alkyl, C₁-C₈-alkenyl, C₁-C₈-heteroalkyl, C₁-C₈-heteroalkenyl, C₁-C₈-alkoxy, C₁-C₈-alkenoxy, C₁-C₈-acyl, C₃-C₈-cycloalkyl, C₃-C₈-cycloalkenyl, C₅-C₈-aryl, C₅-C₈-heteroaryl, arylalkyl, arylalkenyl, C₅₋₈-aryloxy, heteroarylalkyl, heteroarylalkenyl, heterocycloalkyl, heterocycloalkenyl, carboxamido, acylamino, amidino, adamantyl residue, heteroaryloxy residue, toluene, aniline, benzaldehyde, anisole, benzonitrile, phenol, acetophenone, benzoic acid, xylene, styrene, naphthalene, anthracene, phenanthrene, naphthalene, anthracene, phenanthrene, benzpyrene, pyridine, pyrimidine, purine, pyrrolidine, tetrahydrofuran, tetrahydrothiophene, tetrahydropyran, piperidine, pyrrole, furan, thiophene, pyridine, quinoline, indole, pyrimidine, pyrazine, purine, imidazole, pteridine, acridine, chromane, chromene, and coumarin (chromen-2-one), adamantane, pyrazole, diazole, tetrazole, triazole, wherein one or two substituents selected from R^{1′} and R^{2′} as defined hereinbefore can occur independently of one another at each individual atom of the bridged structure, preferably from 1 to 12 atoms.

In a more greatly preferred embodiment of the following invention, the compounds of formula II are selected from the following structures:

TABLE 2
Catalysts according to the invention of formula II

| | |
|---|---|
| | II 1 |
| 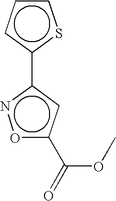 | |
| | II 2 |
| 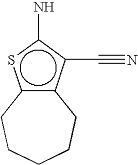 | |
| | II 3 |
| 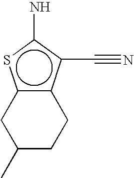 | |
| | II 4 |
| 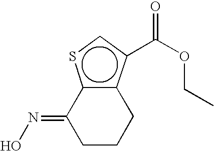 | |
| | II 5 |
| 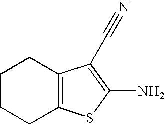 | |
| | II 6 |
| 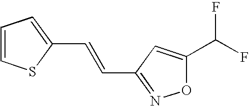 | |

In a further alternative embodiment, the object underlying the present invention is achieved by a method for changing the load state of MHC molecules with ligands that comprises the following steps:
a) providing a composition containing MHC molecules; and
b) adding a catalyst selected from a compound of formula III having the following structure: 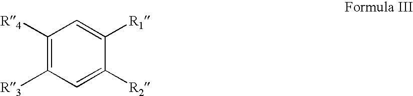
   wherein:
   R^{1″} and R^{2″} can be a bond or are selected independently of one another from a group consisting of:
      H, O, S, N,
      OH, OR^{13″}, SH, SO, SO₂, SO₂R^{13″}, SO₃, HSO₃, SR^{13″}, SR^{13″}R^{14″}, S(CH₂)ₙ(CH₄N);
      X, CX₃, CHX₂, CH₂X, CR^{13″}X₂, CR₂^{13″}X wherein X=halogen,
      CN, CO, COOH, COOCH₃, COOR^{13″},
      NH, NH₂, NHR^{13″}, NR^{13″}R^{14″}, NR^{13″}(CO)R^{14″}; NO, NO₂, NOH, CHNOH, NOR^{13″},
      CH₃, (CH₂)ₙ, (CH₂)ₙCH₃, (CH₂)ₙR^{13″}, (CH)ₙCR^{13″}R^{14″}, OCH₃, O(CH₂)ₙ, O(CH₂)ₙCH₃; O(CH₂)ₙR^{13″}, (CH₂)ₙOH, C₄H₂O(CH₃); (C₃H₂NO)(R^{13″}), (O(CH₂)ₙCH(R^{13″})S(O₂)); (C(CH₃)(CH₂)ₙNHC(O)S), ((CH₂)ₙN(CH₂)ₙC(R^{13″})S), (CHC(R^{13″})N(R^{41″})NC(R^{13″}), NR^{13″}(CH₂)ₙR^{14″}, and (C₂H₃N₂O(NR^{13″}R^{14″}),
      wherein n=from 1 to 30, and
      R^{13″} and R^{14″} are selected independently of one another from a group consisting of
      H, O, S, N,
      OH, OR^{15″}, SH, SO, SO₂, SO₃, HSO₃, SR^{15″}, SR^{15″}R^{15″}, SC(CX₃)XCOOR^{15″},
      X, CX₃, CHX₂, CH₂X, CR^{15″}X₂, CR₂^{15″}X wherein X=halogen,
      CN, CO, COOH, COOCH₃, COOR^{15″},
      NH, NH₂, NHR^{13″}, NR^{15″}R^{16″}, NO, NO₂, NOH, NOR^{15″},
      CH₃, (CH₂)ₙ, (CH₂)ₙCH₃, (CH₂)ₙR^{15″}, OCH₃, O(CH₂)ₙ, O(CH₂)ₙCH₃; O(CH₂)ₙR^{15″}, (CH₂)ₙOH, C₆H₄CH₃, C₆H₉, C₃H₅N₂O₂, (C₃H₂NS)(R^{15″}), and (N(R^{15″}C₃HNO(R^{16″})), CH(R^{15″})(CH₂)ₙR^{16″},
      wherein n=from 1 to 30, and
      R^{15″} and R^{16″} are selected independently of one another from a group consisting of
      H, O, S, N,
      OH, SH, SO, SO₂, SO₃, HSO₃,
      X, CX₃, CHX₂, CH₂X, wherein X=halogen,
      CN, CO, COOH, COOCH₃,
      NH, NH₂, NO, NO₂, NOH,
      CH₃, (CH₂)ₙ, (CH₂)ₙCH₃, OCH₃, O(CH₂)ₙ, O(CH₂)ₙCH₃; and (CH₂)ₙOH, wherein n=from 1 to 30,
      and/or
      R^{1″} and R^{2″} are selected independently of one another from a group consisting of a branched or unbranched C₁-C₃₀-alkyl, C₁-C₃₀-alkenyl, C₁-C₃₀-heteroalkyl, C₁-C₃₀-heteroalkenyl, C₁-C₃₀-alkoxy, C₁-C₃₀-alkenoxy, C₁-C₃₀-acyl, C₃-C₈-cycloalkyl, C₃-C₈-cycloalkenyl, C₅-C₃₀-aryl, C₅-C₃₀-heteroaryl, arylalkyl, arylalkenyl, C₅₋₃₀-aryloxy, heteroarylalkyl, heteroarylalkenyl, heterocycloalkyl, heterocycloalkenyl, carboxamido, acylamino, amidino, adamantyl or heteroaryloxy residue; toluene, aniline, benzaldehyde, anisole, benzonitrile, phenol, acetophenone, benzoic acid, xylene, styrene, naphthalene, anthracene, phenanthrene, naphthalene, anthracene, phenanthrene, benzpyrene, pyridine, pyrimidine, purine, pyrrolidine, tetrahydrofuran, tetrahydrothiophene, tetrahydropyran, piperidine, pyrrole, furan, thiophene, pyridine, quinoline, indole, pyrimidine, pyrazine, purine, imidazole, pteridine, acridine, chromane, chromene, and coumarin (chromen-2-one), diazole, tetrazole, pyrazole, C₃H₂S₂O, saturated or unsaturated C₆₋₈-lactone, and succinimide,
   and/or
   R^{3″} and R^{4″} are as defined for R^{1″} and R^{2″} or can be a bond or are selected independently of one another from a group consisting of:
      H, O, S, N,
      SH, SO, SO₂, SO₃, HSO₃, SR^{13″}, SR^{13″}R^{14″},
      X, in particular Br, CX₃, CHX₂, CH₂X, CR^{13″}X₂, CR₂^{13″}X, CR₃^{13″} wherein X=halogen,
      CN, CO, COOH, COOR^{13″},
      NH, NH₂, NHR^{13″}, NR^{13″}R^{14″}, NO, NO₂, NOH, NOR^{13″},
      CH₃, (CH₂)ₙ, (CH₂)ₙCH₃, (CH₂)ₙR^{13″}, OCH₃, O(CH₂)ₙ, O(CH₂)ₙCH₃; O(CH₂)ₙR^{13″}, (CH₂)ₙOH, C₆H₁₀OH, SO₂CF₃, S(CCH(CH₃)N(OH)NC(CH₃)), (CNONC)NHCH₂(N₄CH), NHC(O)(C₄H₂O(CH₃)), CH₂(C₂N₂H₅(CO)₂), SCFCF₃COOCH₃, SCH₂(C₂NSH(NH₂)), C₃N₂H₃, C(CH₃)C(O)NHC(O)CH₂, C(CH₃)CH₂NHC(O)S, C₆H₅, NHC(O)CHNOH, S(CH₂)₂(C₅H₄N), CHC(CN)(COOCH₃), C₃H₄N, S(C(CH₃)NHNC(CH₃)), NH(C₆H₃N₂O), C₆H₄S(O)₂NH, C₆H₄NHC(O)CH₃, NHC(O)CHNOH, and adamantyl; wherein n=from 1 to 30, and
      R^{13″} and R^{14″} are selected independently of one another from
      H, O, S, N,
      SH, SO, SO₂, SO₃, HSO₃, SR^{15″}, SR^{15″}R^{16″},
      X, in particular Br, CX₃, CHX₂, CH₂X, CR^{15″}X₂, CR₂^{15″}X, CR₃^{15″} wherein X=halogen,
      CN, CO, COOH, COOR^{15″},
      NH, NH₂, NHR^{15″}, NR^{15″}R^{16″}, NO, NO₂, NOH, NOR^{15″},
      CH₃, (CH₂)ₙCH₃, (CH₂)ₙR^{15″}, OCH₃, O(CH₂)ₙ, O(CH₂)ₙCH₃; O(CH₂)ₙR^{15″}, (CH₂)ₙOH, C₆H₁₀OH, SO₂CF₃, S(CCH(CH₃)N(OH)NC(CH₃)), (CNONC)NHCH₂(N₄CH), NHC(O)(C₄H₂O(CH₃)), CH₂(C₂N₂H₅(CO)₂), SCFCF₃COOCH₃, SCH₂(C₂NSH(NH₂)), C₃N₂H₃, C(CH₃)C(O)NHC(O)CH₂, C(CH₃)CH₂NHC(O)S, C₆H₅, NHC(O)CHNOH, S(CH₂)₂(C₅H₄N), CHC(CN)(COOCH₃), C₃H₄N, S(C(CH₃)NHNC(CH₃)), NH(C₆H₃N₂O), C₆H₄S(O)₂NH, C₆H₄NHC(O)CH₃, NHC(O)CHNOH, adamantyl;
      wherein n=from 1 to 30, and
      R^{15″} and R^{16″} are selected independently of one another from a group consisting of
      H, O, S, N,
      SH, SO, SO₂, SO₃, HSO₃,
      X, in particular Br, CX₃, CHX₂, CH₂X, wherein X=halogen,
      CN, CO, COOH, COOCH₃,
      NH, NH₂, NO, NO₂, NOH,
      CH₃, (CH₂)ₙCH₃, OCH₃, O(CH₂)ₙ, O(CH₂)ₙCH₃; (CH₂)ₙOH, C₆H₁₀OH, SO₂CF₃, S(CCH(CH₃)N(OH)NC(CH₃)), (CNONC)NHCH₂(N₄CH), NHC(O)(C₄H₂O(CH₃)), CH₂(C₂N₂H₅(CO)₂), SCFCF₃COOCH₃, SCH₂(C₂NSH(NH₂)), C₃N₂H₃, C(CH₃)C(O)NHC(O)CH₂, C(CH₃)CH₂NHC(O)S, C₆H₅, NHC(O)CHNOH, S(CH₂)₂(C₅H₄N), CHC(CN)(COOCH₃), C₃H₄N, S(C(CH₃)NHNC(CH₃)), NH(C₆H₃N₂O), C₆H₄S(O)₂NH, C₆H₄NHC(O)CH₃, NHC(O)CHNOH, and adamantyl; wherein n=from 1 to 30,
      and/or
      R^{3″} and R^{4″} can be selected independently of one another from a group consisting of a branched or unbranched C₁-C₃₀-alkyl, C₁-C₃₀-alkenyl, C₁-C₃₀-heteroalkyl, C₁-C₃₀-heteroalkenyl, C₁-C₃₀-alkoxy, C₁-C₃₀-alkenoxy, C₁-C₃₀-acyl, C₃-C₈-cycloalkyl, C₃-C₈-cycloalkenyl, C₅-C₃₀-aryl, C₅-C₃₀-heteroaryl, arylalkyl, arylalkenyl, C₅₋₃₀-aryloxy, heteroarylalkyl, heteroarylalkenyl, heterocycloalkyl, heterocycloalkenyl, carboxamido residue, acylamino residue, amidino residue, adamantyl residue, heteroaryloxy residue, toluene, aniline, benzaldehyde, anisole, benzonitrile, phenol, acetophenone, benzoic acid, xylene, styrene, naphthalene, anthracene, phenanthrene, naphthalene, anthracene, phenanthrene, benzpyrene, pyridine, pyrimidine, purine, pyrrolidine, tetrahydrofuran, tetrahydrothiophene, tetrahydropyran, piperidine, pyrrole, furan, thiophene, pyridine, quinoline, indole, pyrimidine, pyrazine, purine, imidazole, pteridine, acridine, chromane, chromene, and coumarin (chromen-2-one), diazole, tetrazole, pyrazole, C₃H₂S₂O, saturated or unsaturated C₆₋₈-lactone, and succinimide;
and/or
R^{3″} and R^{4″} together can form a bridged structure selected from a branched or unbranched C₁-C₈-alkyl, C₁-C₈-alkenyl, C₁-C₈-heteroalkyl, C₁-C₈-heteroalkenyl, C₁-C₈-alkoxy, C₁-C₈-alkenoxy, C₁-C₈-acyl, C₃-C₈-cycloalkyl, C₃-C₈-cycloalkenyl, C₅-C₈-aryl, C₅-C₈-heteroaryl, arylalkyl, arylalkenyl, C₅₋₈-aryloxy, heteroarylalkyl, heteroarylalkenyl, heterocycloalkyl, heterocycloalkenyl, carboxamido, acylamino, amidino, adamantyl residue, heteroaryloxy residue, toluene, aniline, benzaldehyde, anisole, benzonitrile, phenol, acetophenone, benzoic acid, xylene, styrene, naphthalene, anthracene, phenanthrene, naphthalene, anthracene, phenanthrene, benzpyrene, pyridine, pyrimidine, purine, pyrrolidine, tetrahydrofuran, tetrahydrothiophene, tetrahydropyran, piperidine, pyrrole, furan, thiophene, pyridine, quinoline, indole, pyrimidine, pyrazine, purine, imidazole, pteridine, acridine, chromane, chromene, and coumarin (chromen-2-one), diazole, tetrazole, pyrazole, C₃H₂S₂O, saturated or unsaturated C₆₋₈-lactone, and succinimide, wherein one or two substituents selected from R^{1″} and R^{2″} can occur independently of one another at each individual atom of the bridged structure, preferably from 1 to 12 atoms,
c) changing the load state of the MHC molecules; and
d) isolating the MHC molecules whose load state has been changed.

In a particularly preferred embodiment of the present invention, the substituents of formula III are defined as follows:
R^{1″} and R^{2″} can be a bond or can be selected together or independently of one another from a group consisting of:
   H, O, S, N,
   OH, OR^{13″}, SH, SO, SO₂, SO₂R^{13″}, SO₃, HSO₃, SR^{13″}, SR^{13″}R^{14″}, S(CH₂)ₙ(CH₄N);
   X, CX₃, CHX₂, CH₂X, CR^{13″}X₂, CR₂^{13″}X wherein X=halogen, CN, CO, COOH, COOCH₃, COOR^{13″},
   NH, NH₂, NHR^{13″}, NR^{13″}R^{14″}, NR^{13″}(CO)R^{14″}; NO, NO₂, NOH, CHNOH, NOR¹³,
   CH₃, (CH₂)ₙ, (CH₂)ₙCH₃, (CH₂)ₙR^{13″}, (CH)ₙCR^{13″}R^{14″}, OCH₃, O(CH₂)ₙ, O(CH₂)ₙCH₃; O(CH₂)ₙR^{13″}, (CH₂)ₙOH, C₄H₂O(CH₃); (C₃H₂NO)R^{13″}), (O(CH₂)ₙCH(R^{13″})S(O₂)); (C(CH₃)(CH₂)ₙNHC(O)S), ((CH2)ₙN(CH₂)ₙC(R^{13″})S), (CHC(R^{13″})N(R^{14″})NC(R^{13″}), and (C₂H₃N₂O(NR^{13″}R^{14″})),
   wherein n=from 1 to 10, and
   R^{13″} and R^{14″} are selected independently of one another from a group consisting of
   H, O, S, N,
   OH, OR^{15″}, SH, SO, SO₂, SO₃, HSO₃, SR^{15″}, SR^{15″}R^{15″}, SC(CX₃)XCOOR^{15″},
   X, CX₃, CHX₂, CH₂X, CR^{15″}X₂, CR₂^{15″}X wherein X=halogen,
   CN, CO, COOH, COOCH₃, COOR^{15″},
   NH, NH₂, NHR^{13″}, NR^{15″}R^{16″}, NO, NO₂, NOH, NOR^{15″},
   CH₃, (CH₂)ₙ, (CH₂)ₙCH₃, (CH₂)ₙR^{15″}, OCH₃, O(CH₂)ₙ, O(CH₂)ₙCH₃; O(CH₂)ₙR^{15″}, (CH₂)ₙOH, C₆H₄CH₃, C₆H₉, C₃H₅N₂O₂, and (C₃H₂NS)(R^{15″}),
   wherein n=from 1 to 10, and
   R^{15″} and R^{16″} are selected independently of one another from a group consisting of
   H, O, S, N,
   OH, SH, SO, SO₂, SO₃, HSO₃,
   X, CX₃, CHX₂, CH₂X, wherein X=halogen,
   CN, CO, COOH, COOCH₃,
   NH, NH₂, NO, NO₂, NOH,
   CH₃, (CH₂)ₙ, (CH₂)ₙCH₃, OCH₃, O(CH₂)ₙ, O(CH₂)ₙCH₃; and (CH₂)ₙOH,
   wherein n=from 1 to 10,
and/or
R^{3″} and R^{4″} are as defined for R^{1″} or R^{2″} or can be a bond or are selected independently of one another from a group consisting of:
   H, O, S, N,
   SH, SO, SO₂, SO₃, HSO₃, SR^{13″}, SR^{13″}R^{14″},
   X, in particular Br, CX₃, CHX₂, CH₂X, CR^{13″}X₂, CR₂^{13″}X, CR₃^{13″} wherein X=halogen,
   CN, CO, COOH, COOR^{13″},
   NH, NH₂, NHR^{13″}, NR^{13″}R^{14″}, NO, NO₂, NOH, NOR^{13″},
   CH₃, (CH₂)ₙ, (CH₂)ₙCH₃, (CH₂)ₙR^{13″}, OCH₃, O(CH₂)ₙ, O(CH₂)ₙCH₃; O(CH₂)ₙR^{13″}, (CH₂)ₙOH, C₆H₁₀OH, SO₂CF₃, S(CCH(CH₃)N(OH)NC(CH₃)), (CNONC)NHCH₂(N₄CH), NHC(O)(C₄H₂O(CH₃)), CH₂(C₂N₂H₅(CO)₂), SCFCF₃COOCH₃, SCH₂(C₂NSH(NH₂)), C₃N₂H₃, C(CH₃)C(O)NHC(O)CH₂, C(CH₃)CH₂NHC(O)S, C₆H₅, NHC(O)CHNOH, S(CH₂)₂(C₅H₄N), CHC(CN)(COOCH₃), C₃H₄N, S(C(CH₃)NHNC(CH₃)), NH(C₆H₃N₂O), C₆H₄S(O)₂NH, C₆H₄NHC(O)CH₃, NHC(O)CHNOH, adamantyl;
   wherein n=from 1 to 10, and
   R^{13″} and R^{14″} are selected independently of one another from
   H, O, S, N,
   SH, SO, SO₂, SO₃, HSO₃, SR^{15″}, SR^{15″}R^{16″},
   X, in particular Br, CX₃, CHX₂, CH₂X, CR^{15″}X₂, CR₂^{15″}X, CR₃^{15″} wherein X=halogen,
   CN, CO, COOH, COOR^{15″},
   NH, NH₂, NHR^{15″}, NR^{15″}R^{16″}, NO, NO₂, NOH, NOR^{15″},
   CH₃, (CH₂)ₙCH₃, (CH₂)ₙR^{15″}, OCH₃, O(CH₂)ₙ, O(CH₂)ₙCH₃; O(CH₂)ₙR^{15″}, (CH₂)ₙOH, C₆H₁₀OH, SO₂CF₃, S(CCH(CH₃)N(OH)NC(CH₃)), (CNONC)NHCH₂(N₄CH), NHC(O)(C₄H₂O(CH₃)), CH₂(C₂N₂H₅(CO)₂), SCFCF₃COOCH₃, SCH₂(C₂NSH(NH₂)), C₃N₂H₃, C(CH₃)C(O)NHC(O)CH₂, C(CH₃)CH₂NHC(O)S, C₆H₅, NHC(O)CHNOH, S(CH₂)₂(C₅H₄N), CHC(CN)(COOCH₃), C₃H₄N, S(C(CH₃)NHNC(CH₃)), NH(C₆H₃N₂O), C₆H₄S(O)₂NH, C₆H₄NHC(O)CH₃, NHC(O)CHNOH, and adamantyl;
   wherein n=from 1 to 10, and
   R^{15″} and R^{16″} are selected independently of one another from a group consisting of
   H, O, S, N,
   SH, SO, SO₂, SO₃, HSO₃,
   X, in particular Br, CX₃, CHX₂, CH₂X, wherein X=halogen,
   CN, CO, COOH, COOCH₃,
   NH, NH₂, NO, NO₂, NOH,
   CH₃, (CH₂)ₙCH₃, OCH₃, O(CH₂)ₙ, O(CH₂)ₙCH₃; (CH₂)ₙOH, C₆H₁₀OH, SO₂CF₃, S(CCH(CH₃)N(OH)NC(CH₃)), (CNONC)NHCH₂(N₄CH), NHC(O)(C₄H₂O(CH₃)), CH₂(C₂N₂H₅(CO)₂), SCFCF₃COOCH₃, SCH₂(C₂NSH(NH₂)), C₃N₂H₃, C(CH₃)C(O)NHC(O)CH₂, C(CH₃)CH₂NHC(O)S, C₆H₅, NHC(O)CHNOH, S(CH₂)₂(C₅H₄N), CHC(CN)(COOCH₃), C₃H₄N, S(C(CH₃)NHNC(CH₃)), NH(C₆H₃N₂O), C₆H₄S(O)₂NH, C₆H₄NHC(O)CH₃, NHC(O)CHNOH, and adamantyl;
   wherein n=from 1 to 10,
and/or
R^{3″} and R^{4″} together can form a bridged structure selected from a branched or unbranched C₁-C₈-alkyl, C₁-C₈-alkenyl, C₁-C₈-heteroalkyl, C₁-C₈-heteroalkenyl, C₁-C₈-alkoxy, C₁-C₈-alkenoxy, C₁-C₈-acyl, C₃-C₈-cycloalkyl, C₃-C₈-cycloalkenyl, C₅-C₈-aryl, C₅-C₈-heteroaryl, arylalkyl, arylalkenyl, C₅₋₈-aryloxy, heteroarylalkyl, heteroarylalkenyl, heterocycloalkyl, heterocycloalkenyl, carboxamido, acylamino, amidino, adamantyl residue, heteroaryloxy residue, toluene, aniline, benzaldehyde, anisole, benzonitrile, phenol, acetophenone, benzoic acid, xylene, styrene, naphthalene, anthracene, phenanthrene, naphthalene, anthracene, phenanthrene, benzpyrene, pyridine, pyrimidine, purine, pyrrolidine, tetrahydrofuran, tetrahydrothiophene, tetrahydropyran, piperidine, pyrrole, furan, thiophene, pyridine, quinoline, indole, pyrimidine, pyrazine, purine, imidazole, pteridine, acridine, chromane, chromene, and coumarin (chromen-2-one), diazole, tetrazole, pyrazole, C₃H₂S₂O, saturated and unsaturated C₆₋₈-lactone, succinimide, wherein one or two substituents selected from R^{1″} and R^{2″} can occur independently of one another at each individual atom of the bridged structure, preferably from 1 to 12 atoms.

In an even more preferred embodiment, the compounds according to formula III are selected from the following structures:

TABLE 3
Catalysts according to the invention of formula III

| | |
|---|---|
| | III 1 |
| 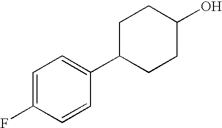 | |
| | III 2 |
| 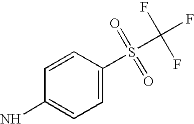 | |
| | III 3 |
| 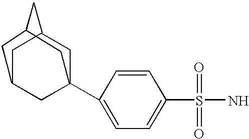 | |
| | III 4 |
| 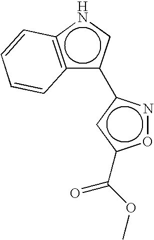 | |
| | III 5 |
| 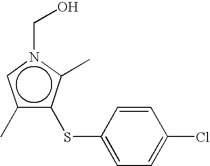 | |
| | III 6 |
| 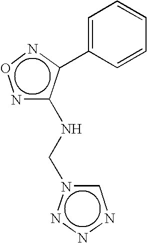 | |
| | III 7 |
| 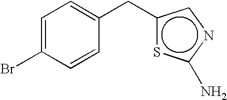 | |
| | III 8 |
| 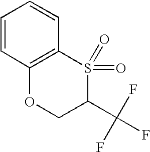 | |
| | III 9 |
| 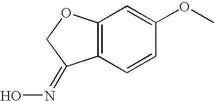 | |
| | III 10 |
| 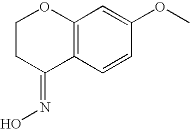 | |
| | III 11 |
| 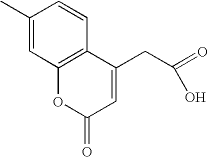 | |
| | III 12 |
| 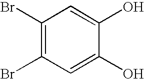 | |
| | III 13 |
| 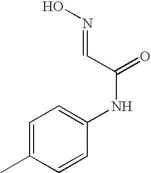 | |
| | III 14 |
| 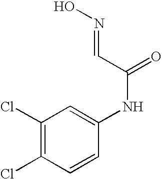 | |
| | III 15 |
| 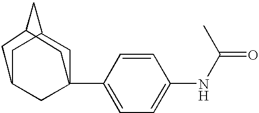 | |
| | III 16 |
| 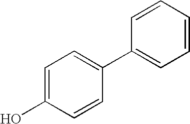 | |
| | III 17 |
| 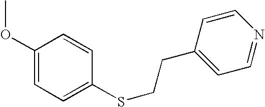 | |
| | III 18 |
| 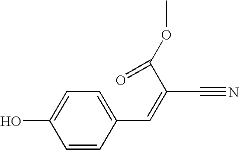 | |
| | III 19 |
| 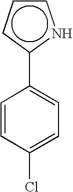 | |
| | III 20 |
| 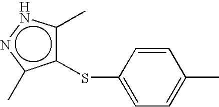 | |
| | III 21 |
| 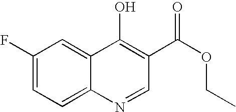 | |
| | III 22 |
| 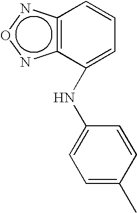 | |
| | III 23 |
| 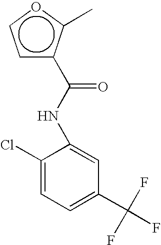 | |
| | III 24 |
| 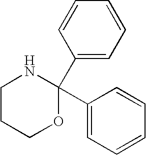 | |
| | III 25 |
| 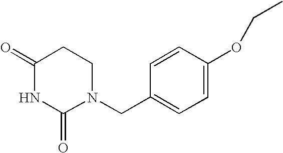 | |
| | III 26 |
| 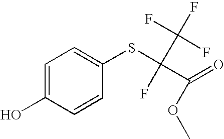 | |
| | III 27 |
| 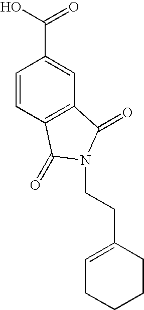 | |
| | III 28 |
| 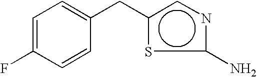 | |
| | III 29 |
| 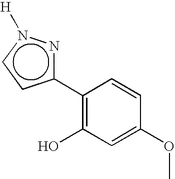 | |
| | III 30 |
| 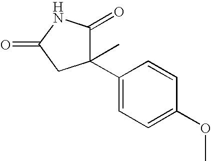 | |
| | III 31 |
| 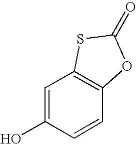 | |
| | III 32 |
| 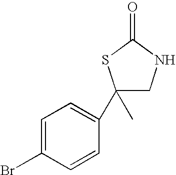 | |
| | III 33 |
| 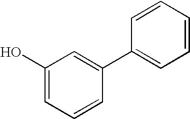 | |
| | III 34 |
| 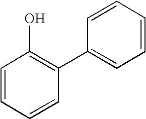 | |
| | III 35 |
| 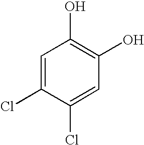 | |
| | III 36 |
| 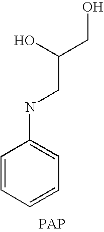 | |
| | III 37 |
| 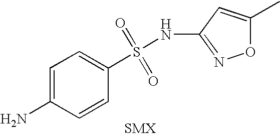 | |

In a further alternative embodiment, the object underlying the present invention is achieved by a method for changing the load state of MHC molecules with ligands that comprises the following steps:
a) providing a composition containing MHC molecules; and

b) adding a catalyst selected from a compound of formulae IV1 to IV3;

TABLE 4
Catalysts according to the invention of formulae IV 1 IV 2 and IV 3

| | |
|---|---|
| | IV 1 |
| 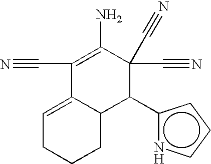 | |
| | IV 2 |
| 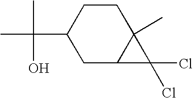 | |
| | IV 3 |
| 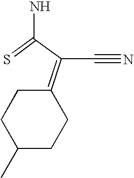 | |

c) changing the load state of the MHC molecules; and
d) isolating the MHC molecules whose load state has been changed.

Method steps (a), (b) and optionally (c) of the above-mentioned methods according to the invention for changing the load state of MHC molecules with ligands can be carried out in any desired sequence or in parallel. For example, steps (b) and (c) can take place in parallel. Also, instead of providing the composition containing MHC molecules, the catalyst, preferably in solution, can first be provided in a method step (a) and then the other substances can be added thereto.

According to the present invention, the above-mentioned substituents are preferably defined as follows:

An alkyl according to the present invention includes linear, branched or cyclic C₁-C₃₀, preferably C₁-C₂₀, more preferably C₁-C₆, hydrocarbon structures as well as combinations thereof. “Lower alkyls” refer to alkyl groups having from 1 to 6 carbon atoms. Examples of lower alkyl groups include methyl, ethyl, propyl, isopropyl, butyl, sec.- and tert.-butyl and the like. Cycloalkyls are a sub-group of the alkyls and include cyclic hydrocarbon groups having from 3 to 8 carbon atoms. Examples of cycloalkyl groups include cyclopropyl, cyclobutyl, cyclopentyl, norbornyl and the like.

An alkenyl according to the present invention includes linear, branched or cyclic unsaturated C₁-C₃₀, preferably C₁-C₂₀, more preferably C₁-C₆, hydrocarbon structures as well as combinations thereof. “Lower alkenyls” refer to alkenyl groups having from 1 to 6 carbon atoms. Examples of lower alkenyl groups include methenyl, ethenyl, propenyl, isopropenyl, butenyl, sec.- and tert.-butenyl and the like. Cycloalkenyls are a sub-group of the alkenyls and include cyclic hydrocarbon groups having from 3 to 20 carbon atoms, preferably from 3 to 8 carbon atoms. Examples of cycloalkenyl groups include cyclopropenyl, cyclobutenyl, cyclopentenyl and the like.

A heteroalkyl or heteroalkenyl of the present invention includes an alkyl or alkenyl as defined above in which one or two carbon atoms has/have been replaced by a hetero atom such as, for example, oxygen, nitrogen or sulfur.

Alkoxy or alkoxyl groups according to the present invention refer to groups of from 1 to 30 carbon atoms, preferably from 1 to 20 carbon atoms, more preferably from 1 to 8 carbon atoms, in an unbranched, branched or cyclic configuration, as well as combinations thereof, which are linked to the main compound via an oxygen atom. Examples include methoxy, ethoxy, propoxy, isopropoxy, cyclopropyloxy, cyclohexyloxy and similar groups. Lower alkoxy or alkoxyl groups refer to groups having from 1 to 4 carbon atoms.

Alkenoxy or alkenoxyl groups according to the present invention refer to groups of from 1 to 30 carbon atoms, preferably from 1 to 20 carbon atoms, more preferably from 1 to 8 carbon atoms, in an unbranched, branched or cyclic unsaturated configuration, as well as combinations thereof, which are linked to the main compound via an oxygen atom.

Examples include methenoxy, ethenoxy, propenoxy, isopropenoxy, cyclopropenyloxy, cyclohexenyloxy and the like. Lower alkenoxy or alkenoxyl groups refer to groups having from 1 to 4 carbon atoms.

Aryls and heteroaryls according to the present invention refer to a 5- or 6-membered aromatic or heteroaromatic ring containing from 0 to 3 hetero atoms selected from O, N and S; a bicyclic 9- or 10-membered aromatic or heteroaromatic ring system containing from 0 to 5 hetero atoms selected from O, N and S; or a tricyclic 13- or 14-membered aromatic or heteroaromatic ring system containing from 0 to 7 hetero atoms selected from O, N and S. The aromatic 6- to 14-membered ring systems include, for example, benzene, naphthalene, indane, tetralin and fluorene, and the 5- to 10-membered aromatic heterocyclic rings include, for example, imidazole, pyridine, indole, thiophene, benzopyranone, thiazole, furan, benzimidazole, quinoline, isoquinoline, quinoxaline, pyrimidine, pyrazine, tetrazole and pyrazole.

An arylalkyl or aralkyl according to the present invention means an alkyl residue as defined above that is bonded to an aryl as defined above. Examples of aralkyl residues are benzyl, phenethyl and the like. Heteroarylalkyl means an alkyl residue as defined above that is bonded to a heteroaryl ring as defined above. Heteroarylalkenyl means an alkenyl residue as defined above that is bonded to a heteroaryl ring as defined above. Examples include pyridinylmethyl, pyrimidinylethyl and the like.

A heterocycle according to the present invention represents a cycloalkyl or aryl residue as defined above in which one or two carbon atoms have been replaced by a hetero atom such as, for example, oxygen, nitrogen or sulfur.

Heteroaryls form a sub-group of the heterocycles. Examples of heterocycles that fall within the scope of protection of this invention include pyrrolidine, pyrazole, pyrrole, indole, quinoline, isoquinoline, tetrahydroisoquinoline, benzofuran, benzodioxane, benzodioxole (which is generally referred to as methylenedioxyphenyl when it occurs as a substituent), tetrazole, morpholine, thiazole, pyridine, pyridazine, pyrimidine, thiophene, furan, oxazole, oxazoline, isoxazole, dioxane, tetrahydrofuran and the like.

Acyls according to the present invention refer to groups of from 1 to 30, preferably from 1 to 20, carbon atoms of the form RCO, more preferably of from 1 to 8 carbon atoms, wherein R can be any of the above-mentioned groups. Examples of acyl groups according to the present invention are methanoyl, acetyl, ethanoyl, propanoyl, butanoyl, malonyl, benzoyl and the like.

All alkyl, alkenyl, heteroalkyl, heteroalkenyl, alkoxy, alkenoxy, acyl, cycloalkyl, cycloalkenyl, aryl, heteroaryl, arylalkyl, arylalkenyl, aryloxy, heteroarylalkyl, heteroarylalkenyl, heterocycloalkyl, heterocycloalkenyl, carboxamido, acylamino, amidino, adamantyl or heteroaryloxy residues according to the present invention can be substituted. Substituted alkyl, alkenyl, heteroalkyl, heteroalkenyl, alkoxy, alkenoxy, acyl, cycloalkyl, cycloalkenyl, aryl, heteroaryl, arylalkyl, arylalkenyl, aryloxy, heteroarylalkyl, heteroarylalkenyl, heterocycloalkyl, heterocycloalkenyl, carboxamido, acylamino, amidino, adamantyl or heteroaryloxy residues refer to alkyl, alkenyl, heteroalkyl, heteroalkenyl, alkoxy, alkenoxy, acyl, cycloalkyl, cycloalkenyl, aryl, heteroaryl, arylalkyl, arylalkenyl, aryloxy, heteroarylalkyl, heteroarylalkenyl, heterocycloalkyl, heterocycloalkenyl, carboxamido, acylamino, amidino, adamantyl or heteroaryloxy residues wherein from 1 to 10 hydrogen atoms can be substituted by SH, SO, SO₂, SO₃, HSO₃, SR¹, SR^{1″}R^{2″}, X, in particular Br, CX₃, CHX₂, CH₂X, CR^{1″}X₂, CR₂^{1″}X, CR₃^{1″}CN, CO, COOH, COOR^{1″}, NH, NH₂, NHR^{1″}, NR^{1″}R^{2″}, NO, NO₂, NOH, NOR^{1″}, CH₃, (CH₂)ₙ, (CH₂)ₙCH₃; (CH₂)ₙR^{1″}, OCH₃, O(CH₂)ₙ, O(CH₂)ₙCH₃; O(CH₂)ₙR^{1″}, (CH₂)ₙOH, a residue lower alkyl, carboxy, carboalkoxy, carboxamido, cyano, carbonyl, alkylthio, sulfoxide, sulfone, acylamino, amidino, phenyl, benzyl, heteroaryl, phenoxy, benzyloxy, heteroaryloxy, or a substituted phenyl, benzyl, heteroaryl, phenoxy, benzyloxy residue, or a heteroaryloxy residue.

A halogen X typically includes the halogens F, Cl, Br and I. Alternatively, instead of the mentioned halogens, the pseudohalogens CN or CO can also be used.

All the compounds described according to the present invention can contain one or more centres of asymmetry and therefore form enantiomers, diastereoisomers and other stereoisomeric forms, which are denoted (R) or (S) according to the terms of absolute stereochemistry. The present invention includes such possible isomers, as well as pure and racemic forms thereof. Optically active (R)- or (S)-isomers can be prepared using Synthosan or chiral reagents or can be separated using standard methods. If the compounds described herein contain olefinic double bonds or other centres of geometric asymmetry, both geometric E and Z isomers are included, unless described otherwise. By analogy, all tautomeric forms are included according to the invention.

Compounds of formulae I, IA, II, III and IV1 to IV3 can be isolated from substance libraries. In the Examples of the present invention, small molecules, for example, from a substance library containing 20,000 small molecules from Chemical Diversity Labs Inc., San Diego, USA were screened. Alternatively, for the compounds used in the methods according to the invention it is possible to use substances from any other substance library that contains compounds of formulae I, IA, II, III and IV1 to IV3, for example substance libraries of the Cambridge Small Compound Library, of the Aldrich Library of Rare Chemicals or substance libraries from Reaction Biology Corp. (RBC), ActiMol, AnalytiCon Discovery, Biofocus, BIOMOL Research Laboratories, Chembridge, Comgenex, Microsource/MSDI, Polyphor, Prestwick Chemical, SPECS and Biospecs, TimTec, Tripos, etc.

MHC molecules used in a method according to the present invention can be obtained from various natural sources or by means of recombinant methods. Natural sources of MHC molecules within the scope of the present invention are, for example, cells or tissue of human or animal origin, more preferably endogenous or non-endogenous dendritic cells, such as, for example, maturated and non-maturated dendritic cells, as well as B-cells or macrophages or other antigen-presenting cells. Natural sources of MHC molecules within the scope of the present invention likewise include preferably body fluids containing the above-defined cells, for example blood, lymph or tissue, etc.

MHC molecules obtained from natural sources and used in a method according to the present invention can be employed in isolated form or together with the cells with which they are associated.

On the one hand, MHC molecules can be obtained from natural sources, i.e. from cells, body fluid, lymph, etc., and isolated by biochemical purification methods, for example chromatographic methods, centrifugation methods, dialysis methods, antibody binding methods, etc. This is carried out, for example, by cleavage of the extracellular portion of the cells contained therein by washing steps and/or cleavage with proteases from the cells contained, for example, in body fluids or lymph and associated with MHC molecules. The MHC molecules are then preferably isolated from the cleaved extracellular fraction by biochemical purification methods, for example by antibody binding methods. The isolated MHC molecules so obtained can then be used in a method according to the invention.

Alternatively, MHC molecules from natural sources can be used in a method according to the invention together with their associated cells, without further isolation of the MHC molecules. To this end, the cells are typically isolated as such together with the MHC molecules. Methods of obtaining cells are known to a person skilled in the art. For example, in order to obtain MHC molecules within the scope of the present invention from natural sources, there are taken from a patient preferably dendritic cells, particularly preferably maturated and/or non-maturated dendritic cells, or B-cells or macrophages, or other cells expressing MHC molecules, individually or in collections of a plurality of cells, and are purified. Purification methods for cells are likewise known from the art to a person skilled in the art. For example, for the purification of cells, the constituents of a cell or tissue sample from a natural source are subjected to separation on the basis of their density. Separation on the basis of density can be carried out by means of chromatographic methods, for example by size-exclusion chromatography or FPLC, or by means of centrifugation, preferably density gradient centrifugation, for example over a Ficoll separating solution. After separation of the constituents on the basis of their density, the cells are typically concentrated by adherence of the desired target cells to a matrix or a solid phase, for example to nylon wool, and undesired cells are depleted. A solid phase or a matrix within the scope of the present method is any surface to which MHC molecules can be bound directly, via a linker, labelling or via their affinity. Alternatively, the cells can be concentrated by chromatographic methods, for example by chromatographic size-exclusion methods or FPLC methods. If necessary, the cell count present in the cell suspension can be determined after a first concentration operation. The cells are then typically separated from the resulting cell suspension by chromatographic methods, by binding to a solid phase, by magnetic sorting by means of a MACS method (Magnetic Activated Cell Sort) or by comparable methods. Magnetic cell sorting permits, for example, the quantitative yield of highly pure cell populations from different tissues. The cells are preferably negatively sorted during separation, i.e. all the cells that are not desired are depleted from the cell mixture. Alternatively, the desired cells can likewise be sorted preferably positively directly from the cell mixture. In both cases, the cells can be labelled preferably with a monoclonal antibody specific for the cell type, which antibody can be bound to a solid phase or to particles, for example to superferromagnetic particles (microbeads). Alternatively, a first specific monoclonal antibody can be recognised by a second antibody which is bound to a solid phase, conventionally microbeads, and recognises the Fc fraction of the first antibody. After a chromatographic method or binding to a solid phase, the cells can be separated from the column or the solid phase, typically by elution. After magnetic sorting by means of a MACS method and binding to microbeads, the cells can typically be separated in a magnetic field over a column. Details relating to the method are evident from the information given by the manufacturer of the microbeads that are used (e.g. Miltenyi Biotech, Bergisch Gladbach, Germany). Purification of the cells is typically carried out at any time, while cooling, preferably while cooling with ice. If required, the cell suspensions obtained are washed at any time during the purification, once or several times, with suitable buffer solutions, for example PBS. When purification is complete, the resulting cells are either stored or used directly. The cells are conventionally stored in a buffer which maintains the cells under physiological conditions and does not cause osmotic pressure. Suitable buffers are, for example, PBS buffer, BSA-PBS buffer (PBS buffer with bovine serum albumin (BSA)), FCS-PBS buffer (PBS buffer with foetal calf serum (FCS)), phosphate buffer, TRIS-HCl buffer, tris-phosphate buffer, or further suitable buffers, such as Dulbecco's modified Eagle Medium (DMEM) or RPMI with 5-10% FCS, the two last-mentioned buffers being suitable in particular for the storage of cells. The cells can optionally be stored and/or used in nutrient medium, for example FCS/10% DMSO.

MHC molecules for use in one of the methods according to the invention can also be made available by recombinant methods. Methods for the recombinant preparation of MHC molecules are known to a person skilled in the art (see, for example, J. Sambrook; E. F. Fritsch; T. Maniatis, 2001, “Molecular cloning: a laboratory manual”, Cold Spring Harbor, N.Y.: Cold Spring Harbor Laboratory Press). To this end, vectors that code for MHC molecules are typically introduced into suitable cell expression systems that express MHC molecules and are then harvested, and the MHC molecules are purified by means of biochemical purification methods, for example chromatographic methods, centrifugation methods, dialysis methods, etc.

MHC molecules used in a method according to the invention are typically MHC monomers or multimers, preferably MHC monomers or multimers of classes I and II. MHC molecules obtained by means of a recombinant method are typically in monomeric form. MHC molecules obtained from a natural source are likewise conventionally in monomeric form. In order to convert such monomeric MHC molecules into a multimeric form, MHC molecules isolated from natural sources or prepared by recombinant methods can be biotinylated and then bound. Binding of the biotinylated MHC molecules to fluorescent-labelled streptavidin molecules yields multimeric MHC complexes, preferably tetrameric complexes, because there are 4 biotin binding sites on streptavidin. Alternatively, MHC molecules isolated from natural sources or prepared by recombinant methods can be multimerised, preferably to give MHC tetramers or pentamers of classes I and II. To this end, a self-arranging coiled-coil domain is preferably co-expressed together with a recombinant MHC molecule. Alternatively to the multimerisation methods described here, any further known methods for the multimerisation of MHC molecules can be used. Such methods are known to a person skilled in the art. A further multimerisation possibility consists in the covalent or non-covalent binding of the molecules to surfaces, such as, for example, culture plates or so-called microbeads. Both are already used for stimulating T-cells, it being possible to use microbeads, in the case where they contain an iron core (e.g. Dynabeads, Dynal Biotech GMBH), also for the selective purification of the cells by means of magnets. For the latter purposes, it is also possible to use monomeric MHC molecules coupled to so-called nano-beads (e.g. Miltenyi Biotec). Multimeric MHC molecules can likewise be obtained commercially. For example, tetramers can be obtained from Becton Dickinson, Beckman Coulter, and pentamers can be obtained from Proimmune. MHC multimers loaded with ligands by a method according to the present invention are preferably capable of binding selectively to T-cells whose T-cell receptor specifically recognises the ligand loaded on the complex.

MHC molecules used in a method according to the invention can be labelled. The MHC molecules whose load state is changed according to a method of the present invention, preferably MHC multimers, are preferably labelled before their load state is changed. The MHC molecules preferably contain labels permitting the generation of a signal that can be detected directly or indirectly. The following modifications are known to the person skilled in the art:
(i) radioactive modifications, e.g. radioactive phosphorylation or radioactive labelling with sulfur, hydrogen, carbon, nitrogen,
(ii) coloured groups (e.g. digoxygenin, etc.),
(iii) fluorescent groups (e.g. fluorescein, etc.),
(iv) chemoluminescent groups,
(v) groups for immobilisation on a solid phase (e.g. biotin, Strep tag, antibodies, antigens, etc.),
   or combinations of modifications according to two or more of the modifications mentioned under (i) to (v).

MHC molecules used in a method according to the invention typically occur in a closed, unloaded, non-receptive conformation or in an open, receptive conformation. In the closed, non-receptive conformation, the MHC molecule is preferably not capable of taking up or giving up ligands. A transition between these different conformations of an MHC molecule typically takes place by a corresponding shift of the equilibrium of the reaction, i.e. a shift of equilibrium between the closed, unloaded, non-receptive conformation; the open, unloaded, receptive conformation; and the open, loaded, receptive conformation. In the methods according to the invention, the MHC molecules are converted by the compounds of formulae I, IA, II, III and IV1 to IV3 that are used preferably from a closed, unloaded, non-receptive conformation to an open, unloaded, receptive conformation. Alternatively, the MHC molecules are converted directly from a closed, unloaded, non-receptive conformation to an open, loaded, receptive conformation. If the MHC molecule is converted into an open, receptive state as a result of the catalytic action of the compounds of formulae I, IA, II, III and IV1 to IV3, or if it is in an open, receptive state, the MHC molecules can be loaded with ligands or a replacement of ligands can take place on the MHC molecule. In other words, the MHC molecule in the open, unloaded, receptive conformation is preferably capable of taking up a ligand. Alternatively, the ligand of an MHC molecule in the open, loaded, receptive conformation can be replaced. In a further alternative, it is also possible to carry out loading of the MHC molecule with ligands directly from the closed, unloaded, non-receptive conformation using the compounds according to the invention of formulae I, IA, II, III and IV1 to IV3.

Monomeric or multimeric MHC molecules used in a method according to the invention can be unloaded or already loaded with ligands. In the case of preparation by recombinant methods, the MHC molecules are typically unloaded after their purification. It is, however, likewise possible for MHC molecules isolated from natural sources or the MHC molecules isolated together with their associated cells to be unloaded after their purification. Such unloaded MHC molecules can be loaded with desired ligands by a method according to the present invention. A change in the load state of MHC molecules with ligands in this case therefore preferably means the loading of (previously unloaded) MHC molecules with ligands. Alternatively, MHC molecules can already be loaded with ligands both after their preparation by recombinant methods and after their isolation from natural sources. The number of ligands on the surface of such loaded MHC molecules can be decreased or the ligands can be removed completely in a method according to the invention. A change in the load state of MHC molecules with ligands in this case therefore preferably means decreasing the load state of (previously loaded) MHC molecules with ligands, and optionally the complete removal of the ligands from (previously loaded) MHC molecules. In a further alternative, ligands of (previously loaded) MHC molecules can be replaced by different desired ligands by means of the methods according to the invention. A change in the load state of MHC molecules with ligands in this case therefore preferably means replacing ligands of (previously loaded) MHC molecules by desired ligands. To this end, ligands already present on the MHC molecule are typically removed or decreased beforehand by a method according to the invention, and then MHC molecules are loaded again with desired ligands by a method according to the invention.

In a preferred embodiment of the method according to the invention, ligands of MHC molecules are native and non-native compounds which bind to MHC molecules under physiological conditions. In this connection, particularly preferred ligands of MHC molecules are antigens, in particular tumour- or pathogen-specific antigens, peptide antigens, tissue-specific self-antigens, antigens of autoreactive T-cells or fragments of such antigens, preferably having a length of from 8 to 25 amino acids, more preferably having a length of from 8 to 15 amino acids. Likewise preferably, ligands of NHC molecules are larger peptide fragments, protein domains, complete proteins, protein mixtures, complex protein mixtures and/or cell lysates, preferably tumour cell lysates.

In a particular embodiment of the object according to the invention, the composition provided in step (a) in a method according to the invention can be an aqueous composition. The composition preferably contains a buffer in addition to the MHC molecules. For example, the following buffers can be used: PBS buffer (for example pH 5.0-8.5, preferably pH 7.0-8.0, 50-200 mM NaCl, preferably about 137.0 mM NaCl, 0.5-5 mM KCl, preferably 2.7 mM KCl, preferably 1-10 mM Na₂HPO₄, preferably 4.3 mM Na₂HPO₄, 0.1-5 mM KHPO₄, preferably 1.4 mM KHPO₄), BSA-PBS buffer (for example pH 5.0-8.5, 1-15 wt. % bovine serum albumin (BSA), preferably pH 5.0-8.0, 50-200 mM NaCl, preferably about 137.0 mM NaCl, 0.5-5 mM KCl, preferably 2.7 mM KCl, preferably 1-10 mM Na₂HPO₄, preferably 4.3 mM Na₂HPO₄, 0.1-5 mM KHPO₄, preferably 1.4 mM KHPO₄), FCS-PBS buffer (for example pH 5.0-8.5, 1-15 wt. % foetal calf serum (FCS), preferably pH 5.0-8.0, 50-200 mM NaCl, preferably about 137.0 mM NaCl, 0.5-5 mM KCl, preferably 2.7 mM KCl, preferably 1-10 mM Na₂HPO₄, preferably 4.3 mM Na₂HPO₄, 0.1-5 mM KHPO₄, preferably 1.4 mM KHPO₄), phosphate buffer (for example pH 5.0-8.5, preferably pH 7.0, 20-80 mM Na₂HPO₄, preferably 57.7 mM Na₂HPO₄, 10-60 mM NaH₂PO₄, preferably 42.3 mM NaH₂PO₄), TRIS-HCl buffer (for example 0.5-3.0 M TRIS with HCl (conc.), preferably 1-1.5 M TRIS adjusted to pH 6.8-8.0 with HCl (conc.)), tris-phosphate buffer (for example 20-80 mM Na₂HPO₄, 10-60 mM NaH₂PO₄, 0.5-3.0 M TRIS adjusted to pH 6.0-8.5 with HCl (conc.)), or further suitable buffers. The mentioned concentrations are preferably the concentrations in the buffer solutions before they are added to the composition. From 0 to 15.0 vol. % are typically added to the composition. The composition provided in the method according to the invention can likewise contain organic solvents. Organic solvents are preferably selected from the following group: DMSO, ethanol, methanol, acetone and the like, particularly preferably from 0.1 to 1% DMSO. The pH of the composition is typically from 6.0 to 8.5, preferably from 6.5 to 7.5. In addition to the aqueous solution or the organic solvent, the composition can optionally contain a polar solvent, for example dimethyl sulfoxide (DMSO), dimethylformamide (DMF), TFE (tetrafluoroethylene), hexamethylphosphoric acid triamide (HMPT), hexamethyl-phosphoramide (HMPA) or the like. The final concentration of the polar solvent in the composition can preferably be from 0.05 to 15 wt. %, more preferably from 0.1 to 5 wt. % and yet more preferably from 0.5 to 2 wt. %. Likewise preferably, the loading is carried out in DMEM or RPMI buffers.

In step (b) of the method according to the invention there is typically added to the provided composition from step (a) a catalyst selected from one of the compounds I, II, III or IV1 to IV3 in a concentration of from 0.001 to 500 mM, preferably in a concentration of from 0.001 to 250 mM, more preferably in a concentration of from 0.001 to 100 mM. The molar ratio MHC molecule to ligand is typically from 0.1:10 to 10:0.1. The molar ratio of ligand to MHC molecule is preferably from 0.5:5 to 5:0.5, particularly preferably from 0.75:2.5 to 2.5:0.75. In a particular embodiment of the method according to the invention, the loading of the MHC molecules with ligands or the ligand replacement of the MHC molecules is preferably carried out at a temperature of from 20 to 40° C., more preferably at a temperature of from 24 to 39° C. and most preferably at a temperature of from 36 to 38° C.

When the load state of MHC molecules is changed with ligands in step (c) by a method according to the invention, the amount of ligands loaded onto or replaced on an MHC molecule by the method according to the invention is preferably controlled. For example, it is possible by means of the method according to the invention, preferably in vitro, to achieve a load state that leads to an equal load, to an increase or alternatively to a decrease in the loading of MHC molecules with ligands as compared with the loading of MHC molecules under in vivo conditions, or as compared with the loading of the MHC molecules contained in the composition before the method according to the invention was carried out. In a particularly preferred embodiment of the method according to the invention, changing the load state of MHC molecules with ligands can lead to the new loading of previously unloaded MHC molecules, to the removal of some or all of the ligands that were already present, or to the replacement of ligands that were already present by different, desired ligands. Methods for determining load states under in vivo and in vitro conditions are known to a person skilled in the art.

In a preferred embodiment of the method according to the invention, a change in the load state of (unloaded) MHC molecules with ligands is achieved in step (c) by addition of potential ligands, in particular peptides, for example peptide ligands, to the composition containing MHC molecules according to method step (a) and compounds of formulae I, IA, II, III and IV1 to IV3 (catalyst) according to method step (b). The concentrations of MHC molecules, ligands and catalysts are preferably as described above. Method steps (a), (b) and (c) of the method according to the invention described here for changing the load state of MHC molecules with ligands can be carried out in any desired sequence or in parallel. For example, steps (b) and (c) can take place in parallel. Also, instead of providing the composition containing MHC molecules, the compounds of formulae I, IA, II, III or IV1 to IV3, preferably in solution, can first be provided in a method step and then the other method steps, such as, for example, addition of ligands and/or MHC molecules, can be carried out. Likewise, all the steps (a), (b) and (c) can be carried out simultaneously.

In another alternative embodiment of the method according to the invention, a change in the load state of MHC molecules already loaded with ligands is achieved in a step (c′) by replacing some or all of the ligands already present on the MHC molecule by different desired ligands. To this end, in a first step (i), a decrease in the load density of ligands on the MHC molecules to a desired load state is preferably achieved (preferably analogously to the alternative method step (c″) described in greater detail hereinbelow). To this end, some or optionally all of the ligands already bound to the MHC molecule are typically removed by a washing step using the compounds of formulae I, IA, II, III or IV1 to IV3 (catalysts). The MHC molecules can thereby be immobilised on a solid phase, for example Sepharose, beads, microbeads, etc., or via linkers obtainable in the art, for example tags such as a His-tag. Any suitable buffer described above can be used in the washing step. Thereafter, in a second step (ii), the desired ligand and optionally a further amount of the compounds of formulae I, IA, II, III or IV1 to IV3 (catalysts) are typically added to the composition and incubated together with the MHC molecule. Alternatively, the desired new ligand can be added to the composition in parallel with step 1 of method step (c′). The new ligand thereby binds to the MHC molecule, preferably by a shift of the concentration equilibrium, i.e. the new ligand is present preferably in such a concentration that displacement of the previously bound ligand by the new ligand takes place using the compounds of formulae I, IA, II, III or IV1 to IV3 (catalysts) takes place. Method steps (a) and (b) and the alternative step (c′) described here of the method according to the invention can therefore be carried out in any desired sequence or in parallel. For example, steps (a), (b) and (c′) can taken place in parallel. Also, instead of providing the composition containing MHC molecules, the catalyst, preferably in solution, can first be provided in a method step (a) and then step (b) can be carried out. Step (c′) is typically carried out after steps (a) and (b) but, as an alternative, can also be carried out in parallel with steps (a) and (b).

In an alternative embodiment of the method according to the invention, a change in the load state of MHC molecules already loaded with ligands in another alternative step (c″) leads to a decrease in the load density of ligands on the MHC molecules. To this end, some or optionally all of the ligands already bound to the MHC molecule are typically removed using the compounds of formulae I, IA, II, III or IV1 to IV3 (catalysts). For this purpose, after providing the composition containing MHC molecules according to method step (a) and addition of the catalysts according to the invention according to method step (b), a washing step is preferably carried out, which step separates from the MHC molecules the ligands detached from the MHC molecule by means of the compounds of formulae I, IA, II, III or IV1 to IV3. To this end, the MHC molecules can be immobilised on a solid phase, for example Sepharose, beads, microbeads, for example via linkers obtainable in the art, for example tags such as a His-tag, etc. Any suitable buffers described above can be used in the washing step. Method steps (a), (b) and (c″) of the embodiment described here of the method according to the invention can be carried out in any desired sequence or in parallel. For example, steps (b) and (c″) can take place in parallel. Also, instead of providing the composition containing MHC molecules, the catalyst, preferably in solution, can first be provided in a method step and then the other steps (b) and (c″), such as, for example, the addition of ligands and/or MHC molecules, can be carried out. Likewise, all the steps (a), (b) and (c″) can be carried out simultaneously.

In a particular embodiment of the method according to the invention, the change in the load state of MHC molecules with ligands in one of steps (c), (c′) or (c″) is typically carried out on an MHC molecule of class I or II, at a peptide binding region of the MHC molecule. The peptide binding region is preferably a peptide binding region of an MHC class I or MHC class II molecule. Typically, the peptide binding region of an MHC I molecule is formed by the α-chain of the MHC class I molecule and consists of a β-sheet formed of eight strands, on which the peptides are clamped between two α-helices and are fixed at both ends by hydrogen bridge bonds between the N- or C-termini and the MHC class I molecule. The peptide binding region of the MHC class I molecule conventionally contains a plurality of binding pockets. The specificity of a ligand at the peptide binding region of an MHC class I molecule is typically produced by binding the amino acid side chains with in each case one of these binding pockets. In the case of the MHC I molecule, the binding pocket is preferably selected from the binding pockets A, B, C, D, E and F, which preferably take up the side chains of the ligands. The peptide binding region of an MHC II molecule is typically formed by the α₁ and β₁ domains of the α- and β-chain forming the MHC II molecules. The peptide binding region of the MHC II molecule likewise conventionally contains one or more binding pockets. In the case of the MHC II molecule, the binding pocket is preferably selected from the binding pockets P1, P3, P4, P6, P7 and P9, which preferably take up the side chains of the ligands. Likewise preferably, the specificity of the ligand at the peptide binding region, or preferably at a peptide binding pocket, is produced by binding of the amino acid side chains with the described binding pocket. Binding of the ligand is conventionally produced in particular by binding to binding pocket P1 of an MHC molecule of class II, which in the case of HLA-DR molecules differs substantially only by the occupancy of the dimorphic residue β86. Binding of the ligands, or the stability of the complexes, can additionally be influenced by further regions which lie outside the actual peptide binding region. For example, pH-dependent destabilisation is influenced considerably by a region which is located beneath the binding groove. At this location there is a histidine residue (His33), which bridges the α₁ domain with the β₁ domain and is influenced directly by protons and possibly also other H donors (Rötzschke et al. PNAS, 2002, 99: 16946-16950).

In a step d) of the method according to the invention, isolation of the MHC molecules loaded with ligands is carried out, for example by a biochemical or biophysical isolation method or by a method of isolating cells, as described hereinbefore.

Biophysical and biochemical detection methods or isolation methods within the scope of the present invention are preferably spectroscopic methods, sequencing reactions, immunological methods or chromatographic methods. Within the scope of the present invention, spectroscopic methods are preferably selected from a NMR, light scattering, Raman, UV, VIS, IR, circular dichroism method, analysis by mass spectrometry (MS) or X-ray structure analysis. A sequencing reaction is understood as meaning preferably peptide or nucleic acid sequencing. Immunological methods are preferably selected from ELISA methods, antibody binding assays, immunofluorescence detection methods or Western blots. Chromatographic methods according to the present invention are preferably adsorption chromatography, distribution chromatography, ion-exchange chromatography, (size-)exclusion chromatography or affinity chromatography. There is further carried out preferably chromatography by means of Ni-NTA agarose, HPLC, FPLC, or reversed-phase (RP) chromatography.

The present invention further provides a method for determining substances that are able to change the load state of MHC molecules. Such a method can be carried out using both a loaded or an unloaded MHC molecule and can be based, for example, on a kinetic measurement of the ligand dissociation and/or ligand binding or on the detection of the underlying conformational changes. Effects that are to be observed experimentally of substances that may change the load state then consist in a method according to the invention for the determination thereof in, for example, an accelerated dissociation of ligands bound to MHC and/or accelerated binding of ligands, in particular peptides, added to unloaded MHC molecules. However, spectroscopic or thermodynamic measuring methods, in particular in conjunction with conformation-specific antibodies, can also be used for determining the activities of test substances and hence for the identification thereof.

If the method according to the invention is to be carried out on the basis of a loaded MHC molecule (embodiment 1), such a method according to the invention for determining substances that change the MHC load, in particular MHCII load, is generally obtained from a method step (a) provision of MHC molecules loaded with ligands, for example in solution or on a surface, for example a metal surface, in particular a gold surface, (b) addition of a substance to be tested, and (c) measurement of the dissociation of the ligands originally located on the MHC molecules. The measurement can be carried out, for example, as mentioned above, by kinetic measurement, or alternatively by concentration measurement of the dissociated ligands and/or of the ligands remaining on the MHC molecules. The concentration measurement can be carried out by appropriate spectroscopic methods (e.g. surface plasmon resonance, which is able to detect the difference between loaded and unloaded MHC molecule) or by, for example, spectroscopically detectable changes between the loaded and unloaded MHC molecules (e.g. changed fluorescence or absorption properties of the bound or non-bound ligand in the complex or of the complex). It is also possible to use microcalorimetric methods or other methods known to the person skilled in the art, in order to detect the changes brought about by the addition of the test substances in method step (b), compared with the initial state.

In the case of a procedure for determining suitable substances without a load (embodiment 2), the method is as follows: in a first method step (a), unloaded MHC molecules, for example in solution or on a surface, for example a metal surface, in particular a gold surface, are provided; in a method step (b1), a substance to be tested is added; and in a method step (b2), a ligand of the MHC molecule is added. Method steps (b1) and (b2) can also be carried out simultaneously, i.e. in such a case a mixture of ligand and test substance is added. In a final method step (c), the loading of the MHC molecules with the ligand is measured by the methods already mentioned above, for example by kinetic measurements, evaluation preferably being made by comparison with the experimental results obtained without addition of the test substance. Suitable test substances can in turn be specified as a result.

In a variant of the two above-mentioned embodiments of the method according to the invention for determining substances having the property of changing the load state of MHC molecules, two or more different test substances, typically in a mixture, can be added to a test batch. This permits a higher throughput of test substances. The one or more test substance(s) that has/have positive activity in a positive test batch must then be identified in a subsequent method step. In another variant, methods according to the invention can be carried out in the manner of a competition assay. To this end, embodiment 1, for example, is so modified that in method step (b) thereof, not only the test substance is added but, before, at the same time as or after the addition of the test substance, a second ligand for the MHC molecule is added to the test batch. Then, typically by means of the above-described methods, for example the concentration of the second ligand on the MHC molecules or the kinetics of the binding of the second ligand to the MHC molecule is determined. The activity of the test substance is therefore in turn derived from a comparison of the tests without addition of the test substance and after addition of the test substance.

The present invention relates further to the use according to the invention of the compounds of formulae I, IA, II, III or IV1 to IV3, alone or optionally together with one or more ligands, for example peptides, for the preparation of vaccines. In an alternative embodiment, compounds of formulae I, IA, II, III or IV1 to IV3 can be used together with ligands, for example peptides, proteins or antigen-containing cell extracts, as a peptide vaccine for the preparation of vaccines, in particular peptide vaccines. In another alternative embodiment, MHC molecules whose load state has been changed can be used for the preparation of vaccines.

The present invention also provides vaccines. In an embodiment, the vaccines of the present invention typically comprise compounds of formulae I, IA, II, III or IV1 to IV3 (catalysts), alone or optionally together with one or more ligands, for example peptides, especially antigenic peptides, in particular peptides of tumour antigens or of surface proteins of pathogens, for example peptides of viral or bacterial surface proteins. The compounds of formulae I, IA, II, III or IV1 to IV3 can thereby be incorporated in the form of adjuvants into a vaccine according to the invention. The adjuvant properties of the above-mentioned compounds are based on the ability of the above-mentioned compounds according to the invention to change the load state of the patient's own MHC molecules and thereby increase the activity of the ligands used for vaccination. In this respect, therefore, the above-mentioned compounds of formulae I, IA, II, III or IV1 to IV3 fulfil the typical adjuvant properties, which consist in further enhancing the immune response, for example to tumours or to pathogens, effected by the ligands, by, in the present case, increasing the relevant ligand load of a vaccine according to the invention to the MHC-expressing immune cells of the vaccinated patient. Further substances can also optionally be incorporated as adjuvants into a vaccine according to the invention; advantageously, one or more further adjuvants will be chosen depending on the immunogenity and other properties of the antigenic ligand in the vaccine according to the invention. In the case of weak immunogenity in particular, complete Freund's adjuvant can be used. Instead of or in combination with Freund's adjuvant, it is possible (in addition to the compounds of formulae I, IA, II, III or IV1 to IV3) to select adjuvants from, for example, TDM, MDP, muramyl dipeptide, alum solution, CpG oligonucleotides or pluronics. Finally, the antigenic ligand can also be coupled, for example chemically, to KLH (Keyhole Limpet Haemocyanin), a very immunogenic foreign protein, or alternatively KLH can be present in a vaccine according to the invention without chemical coupling to the ligand. Furthermore, it is also possible for cytokines, in particular interferons, for example IFN-gamma, or GM-CSF, M-CSF or G-CSF, to be present as adjuvants in the vaccine.

Such vaccines can be used in particular in the indications mentioned hereinbelow, especially in anti-cancer or anti-infection therapies, and can be administered to a patient in vitro or in vivo. For example, such a vaccine according to the invention can be administered directly to a patient intravenously or subcutaneously. The change in the load state of MHC molecules with ligands preferably takes place in vivo, i.e. the loading with ligands, the replacement of ligands or the decrease in ligands on MHC molecules typically takes place in the patient, by administration of the vaccine. Such vaccines according to the invention can likewise be administered by means of a dialysis method, in which case the change in the load state of MHC molecules likewise preferably takes place in vivo. As an alternative to loading in vivo, cells, body fluids, etc., for example from the lymph, can be taken from the patient, and a vaccine according to the invention can be added thereto in vitro. The change in the load state of the MHC molecules associated with these cells, body fluids, etc. likewise preferably takes place in vitro. After the change in the load state of the MHC molecules in vitro, the cells can be returned to the patient, for example by means of a dialysis method or by intravenous or subcutaneous injection.

In an alternative embodiment, vaccines can contain MHC molecules loaded with ligands or unloaded MHC molecules, the load state of which molecules has preferably been changed by a method according to the invention, optionally together with one or more ligands and/or with one of the compounds of formulae I, IA, II, III or IV1 to IV3. In a particular embodiment of this alternative, vaccines can contain compounds of formulae I, IA, II, III or IV1 to IV3 as well as, in addition, MHC molecules loaded with ligands or unloaded MHC molecules whose load state has been changed preferably by a method according to the invention. In another preferred embodiment of this alternative, vaccines can contain compounds of formulae I, IA, II, III or IV1 to IV3, ligands and, in addition, MHC molecules loaded with ligands or unloaded MHC molecules whose load state has been changed by a method according to the invention. There are preferably used for the vaccines according to the invention those MHC molecules whose load state has been changed by a method according to the invention. These are, for example, MHC molecules that have been loaded with peptides by a method according to the invention, especially with antigenic peptides, in particular peptides of a tumour antigen, or peptides that trigger an immune response under physiological conditions. The presence of ligands and/or catalysts together with the MHC molecules already contained in the vaccine allows the load density of the MHC molecules in the vaccine to be controlled by means of a corresponding adjustment of the equilibrium in the solution. This is of interest in particular when vaccines are not used immediately after being prepared but are first stored. Furthermore, by means of such a vaccine, an increased change in the load state of MHC molecules can optionally be achieved in vivo and/or in vitro by means of particular (e.g. desired) ligands.

Vaccines are typically formulated in liquid or solid form. In liquid vaccines, the concentration of the catalysts present is typically in a range as described above, i.e. in a concentration of from 0.001 to 500 mM, preferably in a concentration of from 0.001 to 250 mM, more preferably in a concentration of from 0.001 to 100 mM. The ligands used in the vaccines according to the invention are typically the ligands defined previously in this description. The MHC molecule present in the vaccines is typically used in unloaded form or is loaded prior to use with a ligand as defined above, more preferably with peptide antigens or with fragments of such antigens, preferably having a length of from 8 to 25 amino acids, more preferably having a length of from 8 to 15 amino acids. It is likewise preferred within the scope of the subject-matter according to the invention for the MHC molecule used for the preparation of a vaccine to be an MHC monomer or an MHC multimer as described above. The MHC monomer or MHC multimer is particularly preferably an MHC monomer or MHC multimer, or an MHC tetramer or pentamer, of MHC classes I or II as described above.

In addition to the above-described components of a vaccine, for example MHC molecules loaded with ligands by the methods according to the invention, or compounds according to the invention of formulae I, IA, II, III or IV1 to IV3, together with one or more ligands as defined in the present invention, the vaccines according to the present invention can contain a pharmaceutically acceptable carrier. The choice of a pharmaceutically acceptable carrier is determined in principle by the manner in which the vaccines according to the invention are administered. The vaccines prepared by a method according to the invention can be administered systemically. Routes for administration include transdermal, oral, parenteral, including subcutaneous or intravenous injections, topical and/or intranasal routes.

The present invention relates further to the use of the vaccines prepared by a method according to the invention for therapeutic purposes. To this end, in an embodiment of the present invention, cells or tissue, preferably dendritic cells, particularly preferably maturated and non-maturated dendritic cells, can be taken from a patient beforehand, as already described above, individually or in collections of a plurality of cells, and isolated. The isolation methods used thereby have been described hereinbefore. The MHC molecules, which are obtained by such a method together with endogenous cells of a patient, can subsequently be loaded with ligands, preferably by a method according to the invention, or the ligands already present on the MHC molecule can be replaced by desired ligands, or the number of ligands present can be decreased. Such desired ligands are preferably antigens, more preferably peptide antigens, protein antigens or tumour antigens, in particular from tumour cell lysates. In a further step, the MHC molecules so loaded are preferably returned to the patient together with the dendritic cells in a subsequent vaccination method, for example via reinjection or dialysis. The dendritic cells or MHC molecules so loaded by a method according to the invention then preferably trigger tumour-specific immune responses in the patient, which lead to rejection and destruction of the transformed tissue. In a preferred embodiment of this subject-matter according to the invention, the compounds according to the invention of formulae I, IA, II, III or IV1 to IV3, together with one or more ligands or the MHC molecules whose load state has been changed by a method according to the invention, can be administered to a patient, preferably in the form of a vaccine.

According to a further preferred object of the present invention, the vaccines according to the invention are used for the treatment of indications mentioned by way of example hereinbelow. By means of vaccines according to the present invention it is possible to treat, for example, those disorders or conditions which are associated with various pathologically excessive or absent immune responses. Such vaccines according to the invention are preferably used to trigger tumour-specific or pathogen-specific immune responses. According to another embodiment of this subject-matter according to the invention, such vaccines according to the present invention, which contain, for example, compounds according to the invention of formulae I, IA, II, III and IV1 to IV3, optionally together with one or more ligands, or optionally the MHC molecules loaded with ligands by a method according to the invention, preferably MHC multimers loaded with ligands, particularly preferably MHC multimers of class II loaded with ligands, in which the load state has been lowered or in which the ligands have been removed or replaced, can be used to attenuate aggressive immune reactions. In an embodiment that is likewise preferred, antigen-presenting cells (APC) are loaded with ligands in order to trigger, attenuate or suppress immune responses. In an even more preferred embodiment, the antigen-presenting cells are selected, for example, from endogenous or non-endogenous maturated and non-maturated dendritic cells, IDO+DC cells, B-cells or macrophages. In a further embodiment of the present invention, the MHC molecules loaded with ligands by a method according to the invention can be used for the treatment of cancer, preferably selected from colon carcinomas, melanomas, renal carcinomas, lymphomas, acute myeloid leukaemia (AML), acute lymphoid leukaemia (ALL), chronic myeloid leukaemia (CML), chronic lymphocytic leukaemia (CLL), gastrointestinal tumours, pulmonary carcinomas, gliomas, thyroid tumours, mammary tumours, prostate tumours, hepatomas, various virus-induced tumours such as, for example, papilloma virus-induced carcinomas (e.g. cervical carcinoma), adenocarcinomas, herpes virus-induced tumours (e.g. Burkitt's lymphoma, EBV-induced B-cell lymphoma), heptatitis B-induced tumours (hepatocell carcinomas), HTLV-1- and HTLV-2-induced lymphomas, acoustic neuroma, cervical cancer, lung cancer, pharyngeal cancer, anal carcinoma, glioblastoma, lymphoma, rectal carcinoma, astrocytoma, brain tumours, stomach cancer, retinoblastoma, basalioma, brain metastases, medulloblastomas, vaginal cancer, pancreatic cancer, testicular cancer, melanoma, thyroidal carcinoma, bladder cancer, Hodgkin's syndrome, meningiomas, Schneeberger disease, bronchial carcinoma, hypophysis tumour, Mycosis fungoides, oesophageal cancer, breast cancer, carcinoids, neurinoma, spinalioma, Burkitt's lymphoma, laryngeal cancer, renal cancer, thymoma, corpus carcinoma, bone cancer, non-Hodgkin's lymphomas, urethral cancer, CUP syndrome, head/neck tumours, oligodendroglioma, vulval cancer, intestinal cancer, colon carcinoma, oesophageal carcinoma, wart involvement, tumours of the small intestine, craniopharyngeomas, ovarian carcinoma, abdomen tumours, ovarian cancer, liver cancer, pancreatic carcinoma, cervical carcinoma, endometrial carcinoma, liver metastases, penile cancer, tongue cancer, gall bladder cancer, leukaemia, plasmocytoma, uterine cancer, lid tumour, prostate cancer, etc., or for the treatment of infectious diseases selected from influenza, malaria, SARS, yellow fever, AIDS, Lyme borreliosis, Leishmaniasis, anthrax, meningitis, viral infectious diseases such as AIDS, Condyloma acuminata, hollow warts, Dengue fever, three-day fever, Ebola virus, cold, early summer meningoencephalitis (FSME), flu, shingles, hepatitis, herpes simplex type I, herpes simplex type II, Herpes zoster, influenza, Japanese encephalitis, Lassa fever, Marburg virus, measles, foot-and-mouth disease, mononucleosis, mumps, Norwalk virus infection, Pfeiffer's glandular fever, smallpox, polio (childhood lameness), pseudo-croup, fifth disease, rabies, warts, West Nile fever, chickenpox, cytomegalic virus (CMV), bacterial infectious diseases such as abort (prostate inflammation), anthrax, appendicitis, borreliosis, botulism, Camphylobacter, Chlamydia trachomatis (inflammation of the urethra, conjunctivitis), cholera, diphtheria, donavanosis, epiglottitis, typhus fever, gas gangrene, gonorrhoea, rabbit fever, Helicobacter pylori, whooping cough, climatic bubo, osteomyelitis, Legionnaire's disease, leprosy, listeriosis, pneumonia, meningitis, bacterial meningitis, anthrax, otitis media, Mycoplasma hominis, neonatal sepsis (Chorioamnionitis), noma, paratyphus, plague, Reiter's syndrome, Rocky Mountain spotted fever, Salmonella paratyphus, Salmonella typhus, scarlet fever, syphilis, tetanus, tripper, tsutsugamushi disease, tuberculosis, typhus, vaginitis (colpitis), soft chancre and infectious diseases caused by parasites, protozoa or fungi, such as amoebiasis, bilharziosis, Chagas disease, Echinococcus, fish tapeworm, fish poisoning (Ciguatera), fox tapeworm, athlete's foot, canine tapeworm, candidosis, yeast fungus spots, scabies, cutaneous Leishmaniosis, lambliasis (giardiasis), lice, malaria, microscopy, onchocercosis (river blindness), fungal diseases, bovine tapeworm, schistosomiasis, sleeping sickness, porcine tapeworm, toxoplasmosis, trichomoniasis, trypanosomiasis (sleeping sickness), visceral Leishmaniosis, nappy dermatitis, miniature tapeworm, for the treatment of autoimmune diseases, such as, for example, autoimmune disorders, in particular type I autoimmune disorders or type II autoimmune disorders or type III autoimmune disorders or type IV autoimmune disorders, such as multiple sclerosis (MS), rheumatoid arthritis, diabetes, type I diabetes, systemic lupus erythematosus (SLE), chronic polyarthritis, Basedow's disease, autoimmune forms of chronic hepatitis, Colitis ulcerosa, type I allergic disorders, type II allergic disorders, type III allergic disorders, type IV allergic disorders, fibromyalgia, hair loss, Bechterew's disease, Crohn's disease, Myasthenia gravis, neurodermitis, Polymyalgia rheumatica, progressive systemic sclerosis (PSS), psoriasis, Reiter's syndrome, rheumatic arthritis, psoriasis, vasculitis, etc.

The present invention relates further to the use of MHC molecules loaded with ligands, which can be prepared by a method according to the invention, for the preparation of a pharmaceutical composition for the treatment of the above-mentioned indications.

The present invention further provides pharmaceutical compositions. The pharmaceutical compositions of the present invention typically comprise a safe and effective amount of the compounds according to the invention of formulae I, IA, II, III and IV1 to IV3, preferably as defined above, optionally together with one or more ligands as defined in the present invention, for example peptides, and optionally a pharmaceutically acceptable carrier, as well as further auxiliary substances and additives. Alternatively, pharmaceutical compositions preferably comprise a safe and effective amount of the MHC molecules whose load state has been changed with ligands by a method according to the invention, and optionally a pharmaceutically acceptable carrier, as well as further auxiliary substances and additives. As used here, “safe and effective amount” means an amount of a compound that is sufficient to significantly induce a positive modification of the condition to be treated, but that is sufficiently small to avoid serious side-effects (with a sensible ratio of advantage/risk), within the range of sensible medical discernment. A safe and effective amount of a compound will vary in connection with the particular condition to be treated, as well as the age and physical condition of the patient to be treated, the severity of the condition, the duration of the treatment, the nature of the accompanying therapy, of the particular pharmaceutically acceptable carrier used and similar factors, within the knowledge and experience of the accompanying doctor. The pharmaceutical compositions according to the invention can further be used for human and also for veterinary medical purposes.

In addition to the compounds according to the invention of formulae I, IA, II, III and IV1 to IV3, optionally together with one or more ligands, or alternatively in addition to the MHC molecules whose load state has been changed with ligands by a method according to the invention, the pharmaceutical compositions of the present invention can contain a pharmaceutically suitable carrier. The term “pharmaceutically acceptable carrier” used here preferably includes one or more compatible solid or liquid fillers, or diluents or encapsulating compounds, which are suitable for administration to a person. The term “compatible”, as used here, means that the constituents of the composition are capable of being mixed with the compound and with one another in such a manner that no interaction occurs which would substantially reduce the pharmaceutical effectiveness of the composition under conventional use conditions. Pharmaceutically acceptable carriers must, of course, exhibit sufficiently high purity and sufficiently low toxicity to render them suitable for administration to a person to be treated.

Some examples of compounds that can be used as pharmaceutically acceptable carriers or constituents thereof are sugars, such as, for example, lactose, glucose and sucrose; starches such as, for example, corn starch or potato starch; cellulose and its derivatives, such as, for example, sodium carboxymethylcellulose, ethylcellulose, cellulose acetate; powdered tragacanth; malt; gelatin; talc; solid lubricants, such as, for example, stearic acid, magnesium stearate; calcium sulfate; vegetable oils, such as, for example, peanut oil, cottonseed oil, sesame oil, olive oil, corn oil and oil from Theobroma; polyols, such as, for example, polypropylene glycol, glycerol, sorbitol, mannitol and polyethylene glycol; alginic acid; emulsifiers, such as, for example, the Tweens®; wetting agents, such as, for example, sodium lauryl sulfate; colouring agents; flavouring agents, pharmaceutical carriers; tablet-forming agents; stabilisers; antioxidants; preservatives; pyrogen-free water; isotonic saline and phosphate-buffered solutions.

The choice of a pharmaceutically acceptable carrier which is to be used together with compounds according to the invention of formulae I, IA, II, III and IV1 to IV3, optionally together with one or more ligands, or alternatively additionally together with MHC molecules whose load state has been changed with ligands by a method according to the invention, is determined in principle by the manner in which the MHC molecules loaded with ligands by a method according to the invention are administered. The MHC molecules loaded with ligands by a method according to the invention can be administered systemically. Routes for administration include transdermal, oral, parenteral, including subcutaneous or intravenous injections, topical and/or intranasal routes.

The suitable amount of the compounds according to the invention of formulae I, IA, II, III and IV1 to IV3 that are to be used, optionally together with one or more ligands, or alternatively of the MHC molecules whose load state has been changed with ligands by a method according to the invention, can be determined by routine experiments with animal models. Such models include, without implying any limitation, rabbit, sheep, mouse, rat, dog and non-human primate models.

Preferred unit dose forms for injection include sterile solutions of water, physiological saline or mixtures thereof. The pH of such solutions should be adjusted to about 7.4.

Suitable carriers for injection or for surgical implantation include hydrogels, devices for controlled or delayed release, polylactic acid and collagen matrices.

Suitable pharmaceutically acceptable carriers for topical application include those which are suitable for use in lotions, creams, gels and the like. If the compound is to be administered perorally, tablets, capsules and the like are the preferred unit dose form. The pharmaceutically acceptable carriers for the preparation of unit dose forms which can be used for oral administration are well known in the art. The choice thereof will depend on secondary considerations such as taste, costs and storability, which are not critical for the purposes of the present invention, and can be made without difficulty by a person skilled in the art.

The present invention likewise preferably relates to the use of compounds according to the invention of formulae I, IA, II, III and IV1 to IV3, optionally together with one or more ligands, or alternatively of MHC molecules whose load state has been changed with ligands by a method according to the invention, preferably MHC multimers, for screening methods and diagnostic methods for seeking and identifying new antigens, in particular tumour antigens, pathoantigens and autoantigens, and for detecting specific T-cells, for example autoreactive T-cells or cytotoxic T-cells, or for monitoring a specific T-cell response. Typically, to this end, in a first step there is provided a composition containing compounds according to the invention of formulae I, IA, II, III and IV1 to IV3, optionally together with one or more ligands, or alternatively containing MHC molecules whose load state has been changed with ligands by a method according to the invention. MHC molecules are MHC molecules as defined above. The compositions used here can correspond to the compositions provided in the methods for changing the load state of MHC molecules. The MHC molecules used in the present case can be loaded with ligands or unloaded. The ligands preferably correspond to the ligands defined hereinbefore in the description and are chosen specifically depending on the underlying object. Preferably, the ligands loaded onto MHC molecules are those which lead to an immune response also under physiological conditions. The ligands used and/or identified in the screening method or diagnostic method are preferably ligands as defined above of MHC molecules, more preferably ligands of tumour- or pathogen-specific antigens as well as antigens of autoreactive T-cells. For example, by the targeted binding of antigens to MHC molecules by means of the screening or diagnostic method according to the invention, the formation of specific T-cell receptors or T-cells on that antigen in a patient can be checked. The immune response optionally obtained with the screening can then be brought into direct association with an indication. Typically, by a method according to the invention, ligands are loaded onto the MHC molecule in such an amount that the amount of ligands on the MHC molecules is sufficient to stimulate an immune response, preferably a T-cell response. In a further step, the MHC molecule can be bound to a solid phase or to a matrix. A solid phase or a matrix within the scope of the present method is preferably any surface to which MHC molecules can be bound directly, via a linker, via labelling or via their affinity. The MHC molecules can be labelled as described above for the binding to a solid phase. For the binding to a solid phase or to a matrix, suitable buffers can be added to the composition. Suitable buffers are known to a person skilled in the art. Suitable buffers within the scope of the present method are, for example, PBS buffer, BSA-PBS buffer (PBS buffer with bovine serum albumin (BSA)), FCS-PBS buffer (PBS buffer with foetal calf serum (FCS)), phosphate buffer, TRIS-HCl buffer, tris-phosphate buffer, or further suitable buffers. The concentrations and amounts of the buffers to be used preferably correspond to those mentioned hereinbefore in the description. In a further step, a substance to be investigated, for example a body fluid or a homogenised tissue, preferably blood or tissue, optionally in one of the buffers described hereinbefore, can be added. The physiological binding partners contained in the added substance conventionally bind to the immobilised MHC molecules optionally loaded with ligands by a method according to the invention. After binding of the physiological binding partners that are present to the MHC molecules, the solid phase or matrix is conventionally washed with a buffer, preferably with one of the buffers described hereinbefore. In a particularly preferred embodiment, the physiological binding partner is preferably a T-cell, particularly preferably a T-cell that possesses a high affinity for an antigen, yet more preferably a T-cell having a high affinity for a tumour- or pathogen-specific antigen. Likewise particularly preferably, the immune response produced in a screening method or to be detected in a diagnostic method is an antigen-specific T-cell response. In a final step, the physiological binding partners, bound to the MHC molecules, of the MHC molecules optionally loaded with ligands can be identified and isolated by a biophysical or biochemical detection or isolation method as described above, preferably by means of FACS or MACS. According to an embodiment that is likewise preferred, T-cells can be isolated antigen-specifically by means of MACS in such method using MHC class II-carrying magnetic beads. The steps mentioned in the above-mentioned screening method or diagnostic method can be changed in any desired sequence. Screening methods or diagnostic methods according to the present invention preferably include in vitro T-cell assays, particularly preferably proliferation assays, ELISPOTS, ELISA methods, chromium release assays, high-throughput screening methods (HTS), etc.

A particular embodiment of the screening or diagnostic method according to the invention relates to a method for seeking and identifying new tumour antigens, for detecting specific cytotoxic T-cells or for monitoring a specific T-cell response, comprising the following steps:
a) providing a composition containing MHC molecules whose load state has been changed with ligands by a method according to the invention; and
b) determining the interaction of MHC molecules whose load state has been changed with ligands by a method according to the invention, with a physiological binding partner of the MHC molecules by means of a biochemical or biophysical detection method; and
c) optionally identifying and isolating the physiological binding partner of the MHC molecules whose load state has been changed with ligands by a method according to the invention, by means of a biochemical or biophysical detection or isolation method.

In a further embodiment of the present invention, in a screening method or diagnostic method, the MHC molecules whose load state has been changed with ligands by a method according to the invention, preferably MHC multimers, can be used for the antigen-specific identification of (e.g. autoreactive) T-cells, preferably of tumour-specific, pathogen-specific or autoreactive T-cells. There are used as ligands preferably those antigens which are associated with one of the indications mentioned hereinbefore. Such a diagnostic method is typically carried out ex vivo, for example by adding samples from the patient, for example samples of body fluids, in particular blood or lymph samples, to MHC molecules loaded by means of the catalysts according to the invention with ligands (for example derived from a pathogen antigen or a tumour antigen, for example a fragment thereof). In a subsequent step, the binding of cells in the patient's sample, for example (auto)reactive T-cells, to the loaded, preferably tetrameric, MHC molecules is detected, for example by means of suitable detection methods, in particular by labelling of the MHC molecules and/or ligands, for example by fluorescent labelling or other spectroscopically detectable compounds. The detection can be carried out in particular by means of FACS analysis.

### DESCRIPTION OF THE FIGURES

FIG. 1: shows the catalysis of the loading of soluble HLA-DR1 molecules by phenol and adamantyl compounds. Panel A) shows structural formulae of p-chlorophenol (pCP), 2-(1-adamantyl)-ethanol (AdEtOH) and 3-(1-adamantyl)-5-carbohydrazide pyrazole (AdCaPy), which were used in the experiments according to the invention. Panel B) shows the loading of empty soluble HLA-DR1 molecules with the HA306-318 peptide. The loading was determined using biotinylated peptide in an ELISA. The height of the bars in the bar diagram shown in the left-hand panel represents the number of peptide/MHC complexes formed after 60 minutes. Black bars represent the uncatalysed loading reaction, where (+) represents spontaneous loading and (−) represents the background signal in the absence of the peptide. The height of the grey bars indicates the number of complexes formed in the catalysed reactions, which were carried out in the presence of 250 μM pCP, AdEtOH or AdCaPy. The corresponding dose-activity curves are shown in the right-hand figure. As will clearly be seen from this figure, all three catalysts are capable in principle of accelerating the reaction. Compared with the pCP curve, however, the corresponding curves for the adamantyl compounds are displaced to the left by more than a power of ten, i.e. they are 10 times more active than pCP.

FIG. 2: shows the catalysis of the loading of HLA-DR molecules on the cell surface by means of dose-activity curves of the loading of fibroblast cells with the HA306-318 peptide. To this end, the cells were incubated with the peptide and also with p-chlorophenol (pCP), 2-(1-adamantyl)-ethanol (AdEtOH) and 3-(1-adamantyl)-5-carbohydrazide pyrazole (AdCaPy). By using a biotinylated peptide, it was then possible, after staining of the cells with fluorescent streptavidin, to detect the amount of bound peptide on L57.23 and L243.6, which express HLA-DR1 (DRB1*0101) or HLA-DR4 (DRB1*0401), two MHC class II molecules which are able to present the HLA306-318 peptide. The absence of any staining of the L929 cells indicates that all the catalysts selectively enhance the binding to the MHC molecules. A comparison of the dose-activity curves confirms the result with the soluble MHC molecules (see also FIG. 1). Here too, the tested adamantyl compounds are found to be substantially more active than pCP and accelerate the loading of the cells even at a concentration markedly less than a tenth of the corresponding pCP concentration.

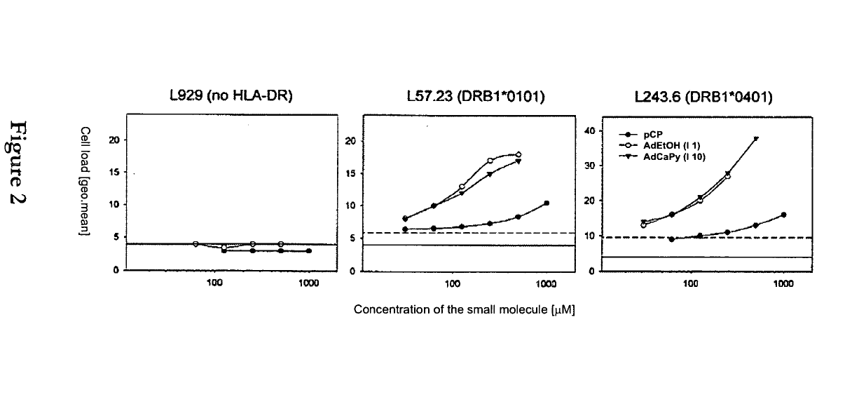

FIG. 3: shows the enhancement of the T-cell response by the catalysis of the loading of HLA-expressing cells by means of the dose-activity curves of the immune response of two different HLA-DR1-restricted T-cells. Either HA306-318 (left-hand panel) or CO260-273 (right-hand panel) was used as peptide antigen. In both cases, the immune response of the T-cells is markedly enhanced by the compounds AdEtOH or AdCaPy used in the method according to the invention. A comparison of the dose-activities shows that the adamantyl compounds are also about 10 to 100 times more effective than pCP in respect of the T-cell response.

FIG. 4: shows the allele-specific effect of the catalysis by adamantyl compounds by a comparison of the dose-activity curves of a HLA-DR4-restricted (left-hand panel) and a HLA-DR2-restricted T-cell response (right-hand panel). HLA-DR4 (DRB1*0401)- and HLADR2 (DRB1*1501)-expressing cells were loaded, as described under Comparison Test 3.4, with HA306-318 or MPB86-100 in the presence of the catalysts and then used in the T-cell assay. While the dose-activity curves of the HLA-DR4-restricted 8475/94 T-cells correspond to those of the HL-DR1-restricted T-cells described hereinbefore in Comparison Test 3.3, a marked difference is to be seen in the case of the curves of the HLA-DR2-restricted 08073 T-cells. In contrast to pCP, which can have an enhancing effect on all the HLA-DR molecules investigated, the adamantyl compounds are evidently active on HLA-DR1 (DRB1*0101) and HLA-DR4 (DRB1*0401), but not on HLADR2 (DRB1*1501). The activity of the adamantyl compound is therefore evidently allele-specific.

FIG. 5: shows a comparison of the dose-activity curves of the catalysis of the binding of MBP86-100 to the HLA-DR2 wild-type molecule (left-hand fig.) and to the mutated HLA-DR2 molecule, in which the valine residue at position 86 of the β-chain has been replaced by glycine (right-hand fig.). While adamantane ethanol is unable to effect catalysis on the unmutated HLA-DR2 molecule of the fibroblast cells, as on the MGAR cells described in Comparison Test 3.4, the V->G substitution has the effect of making the mutated HLA-DR2 molecule receptive to the adamantyl-mediated catalysis again. Because the glycine/valine dimorphism determines the depth of the binding pocket P1, where β86G correlates with a deep pocket, adamantyl compounds evidently mediate their catalytic activity by their binding to a deep P1 pocket.

FIG. 6: shows dose-activity curves of the catalysis of a HLA-DR1 (DRB1*O101)-restricted T-cell response. The experiment was carried out analogously to the description relating to Comparison Test 3.3, the peptide antigen HA306-318 being loaded onto 721.221 cells in order thereby to stimulate EvHA/X5 T-cells. The dose-activity curves show that all the listed adamantyl compounds possess catalytic activity. The different chemical nature of the side chains shows that the activity is mediated substantially by the adamantyl grouping. Because the position of the dose-activity curves is evidently also determined by the side chain, however, at least a modulating activity can be attributed thereto, however.

FIG. 7: shows continuous-flow cytomety measurements of HLA-DR1-restricted human T-cells (PD2) which have been stained with fluorescent-labelled peptide-loaded HLA-DR1 tetramers. The following tetramers were used: HA tetramers, HLA-DR1 tetramers, which were obtained from the manufacturer (Proimmune Ltd.) already loaded with the T-cell antigen HA306-318, and IC tetramers, which carry the non-relevant peptide IC106-120. Panel A) shows continuous-flow cytometry measurements produced by loading IC tetramers with HA306-318 in the presence of adamantyl compounds (AdEtOH). As control, loading with a non-relevant peptide (ABL) was also carried out. Analysis by continuous-flow cytometry after staining of the cells with the complexes revealed that the signal delivered by the PD2 T-cells stained by means of adamantylethanol-loaded tetramers was of a similar height to that delivered by the pre-prepared HA tetramers. Panel B) shows the ligand replacement in the presence and in the absence of adamantylethanol. As will be seen from the bar diagram, in this experiment too, the HA306-318-specific PD2 T-cells are stained with the tetramers catalysed by adamantylethanol. By contrast, no appreciable staining is observed with the complexes in which loading was attempted without catalyst. The complexes formed by means of adamantylethanol additionally exhibit the required antigen specificity, because A 10 T-cells, unlike the PD2, are not stained.

FIG. 8 shows dose-activity curves of further compounds which likewise act via the deep P1 pocket. The structures studied (2-(7,7-dichloro-6-methyl-bicyclo[4.1.0]heptan-3-yl)-propan-2-ol (#4230) and 6-methoxybenzofuran-3(2H)-one-oxime (#3651)) are shown in panel A). Despite very different chemical structures, #4230 at least exhibits a spatial structure which is very similar to that of the adamantyl group. Panel B) shows the activity and allele specificity with the HLA-DR1-restricted HA306-318-specific EvHA/X5 T-cells (left-hand fig.) and the HLA-DR2-restricted MBP86-100-specific 08073 T-cells. On HLA-DR1, #4230 exhibits a catalytic activity that corresponds approximately to that of adamantylethanol. #3651, on the other hand, is weaker but still exhibits higher activity than pCP. On HLA-DR2, both compounds exhibit no activity, for which reason they, similarly to the adamantyl compounds, evidently mediate their catalytic activity by binding to the deep P1 pocket.

FIG. 9 shows dose-activity curves for various catalytically active phenol/aniline compounds. In panel A), structural formulae for some catalytically active phenol and aniline compounds are shown. The catalytic activity of the active compounds of this compound class that have been identified hitherto is in most cases, frequently also markedly, below that of the adamantyl compounds. The mechanism of catalysis is evidently similar to that of the adamantyl compounds, however, because a receptive conformation is initiated here too. B) The catalytic activity of the example substances is demonstrated here by the accelerated loading of soluble HLA-DR1 molecules with HA306-318. A comparison of the dose-activity curves with pCP shows that all the compounds lie within a very similar activity range, DCC and pHDP in particular exhibiting slightly increased activity.

FIG. 10 shows dose-activity curves of the catalysis of HLA-DR loading by pCP and adamantylethanol. The data were determined by means of T-cell assays as described in Comparison Test 3.3 and show the activity of the two catalysts on HLA-DR molecules with a deep or flat P1 pocket {□86G and □86V} as well as on the mouse MHC class II molecules H2-E^{k} and H2-A^{k}. The broken lines show the strength of the uncatalysed T-cell response. As already described in Comparison Test 3.5, the activity of adamantylethanol compared with pCP is greater but is directed towards those HLA-DR molecules which possess the deep P1 pocket, but the activity spectrum of pCP is less specific. There is no dependence on the □86 dimorphism, nor have any other allele-specific limitations of the activity of pCP on HLA-DR molecules been observed hitherto. pCP has no effect on any of the H2-A and H2-E molecules studied hitherto, which are homologues of the human HLA-DQ and HLA-DR molecules.

FIG. 11 shows dose-activity curves of various catalytically active thiophene compounds. Panel A) shows structural formulae of some catalytically active thiophene compounds. The catalytic activity of the active compounds of this compound class that have been identified hitherto is in most cases slightly stronger than that of pCP. The activity of three example compounds is shown here by means of the dose/activity curves of the stimulation of HLA-DR1-restricted EvHA/X5 T-cells. The experiment was carried out as described under Comparison Test 3.3. Panel B) shows the comparative dose-activity curves of pCP, AdEtOH and ATC. As in the case of the active phenol/aniline compounds, the activity here too is evidently independent of the β86 dimorphism of the HAL-DR molecules. As shown in Comparison Test 3.11, the peptide loading both of fibroblasts that express either HLA-DR1 (DRB1*0101; β86G) or HLA-DR2 (DRB1*1501; β86V) is catalysed by the compound ATC. Accordingly, the depth of the P1 pocket evidently plays no part in the case of the thiophene compounds.

FIG. 12 shows the catalytic activity of several adamantyl compounds used according to the invention which come under the generic formulae I and IA. The relative enhancement of the rate of loading is plotted against the concentrations of the catalytic adamantyl compounds used (see Example 3.12). It will clearly be seen that the tested compounds are able to bring about a marked enhancement of the rate of loading, which is dose-dependent. DMSO was used as control. The adamantyl compounds used are shown in the right-hand column of FIG. 12 (with decreasing activity from top to bottom). Their structure is to be found in Table 1.

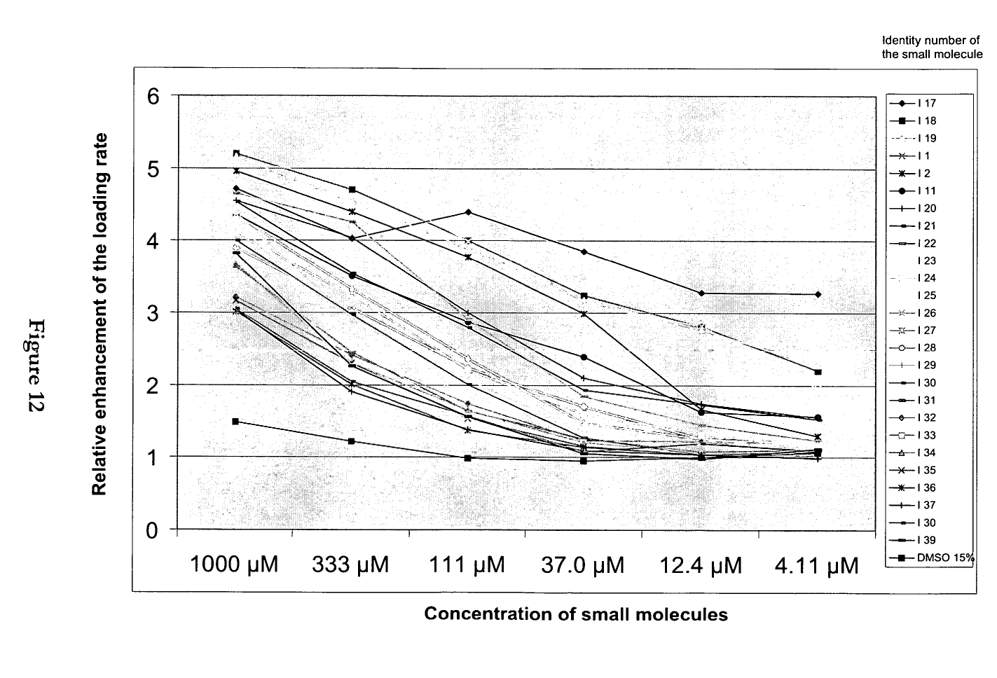

FIG. 13 shows the catalytic activity of several compounds used according to the invention which come under the generic formulae II, III or IV1 to IV3. The relative enhancement of the rate of loading is plotted against the concentrations of the catalytic compounds used (see Example 3.12). It will clearly be seen that the tested compounds are able to bring about a marked enhancement of the rate of loading, which is dose-dependent. DMSO was used as control. The compounds used in the experiments are shown in the right-hand column of FIG. 12 (with decreasing activity from top to bottom). Their structure is to be found in Tables 2 to 4.

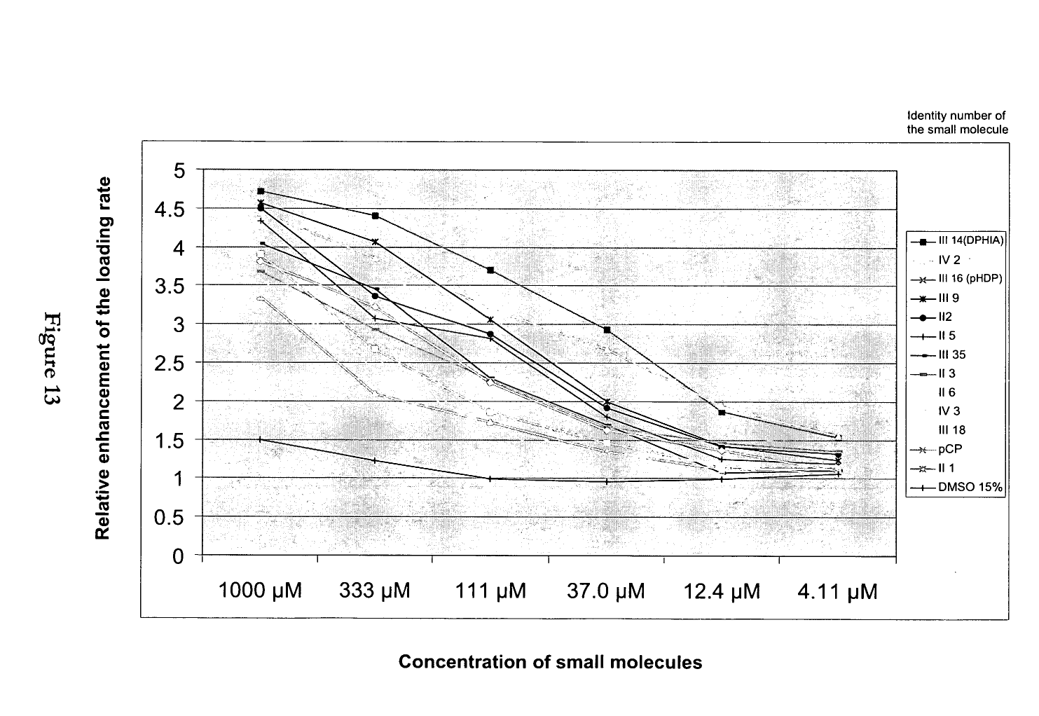

FIG. 14 shows the catalysis of the loading of dendritic cells with antigens for use in adoptive immunotherapies. In the absence of AdEtOH I 1, a compound of generic formulae I or IA, peptide is loaded onto the surface of the DCs to only a small extent (left-hand fig. of FIG. 14A); each point represents an individual cell). A markedly stronger peptide signal is measured, however, when the loading is carried out in the presence of 250 μM AdEtOH (right-hand fig. of FIG. 14A). The enhancement of the antigen-specific T-cell response is also correspondingly high (FIG. 14B). DCs loaded with the antigen in the presence of AdEtOH trigger up to 15 times stronger T-cell responses than DCs loaded without AdEtOH. The level of enhancement shows the expected dependence on the amount of AdEtOH used and is expressed in a marked displacement of the dose/activity curve of the peptide antigen (FIG. 14C). A comparison of the peptide loading with (●) and without (o) AdEtOH is shown. With 250 μM AdEtOH, less than 10 ng/ml of HA306-318 peptide already trigger a T-cell response, which is achieved in the absence of the catalyst only at a concentration of 100 ng/ml of peptide.

FIG. 14 shows that the efficiency of this approach can be increased substantially if the ex vivo antigen loading is enhanced by catalytic compounds such as AdEtOH. Because these MHC class II-expressing cells (DCs) are particularly important in triggering antigen-specific T-cell responses, they are preferably used inter alia in immune therapies. For example, in adoptive tumour immune therapies, DCs from cancer patients are (a) isolated, (b) loaded ex vivo with tumour antigens and then (c) reinjected again in order to trigger tumour-specific immune responses in vivo. Such ex vivo methods of therapy can also be carried out according to the invention, it being possible for compounds of formulae I, IA, II, III or IV1 to IV3 to be used in method step (b). Such a method can—as disclosed in the present application—also be carried out with antigens other than tumour-specific antigens.

FIG. 15 shows the effect of the use of AdEtOH (I 1) as a vaccine additive or adjuvant for enhancing the immune response in vaccination experiments. As the analysis of this experiment makes clear, markedly more T-cells which can react against the ABL domain were evidently activated in the case of the vaccination in which AdEtOH was used as additive. The addition of AdEtOH accordingly resulted in a considerable increase in the efficiency of the vaccination. The number of spots is plotted against vaccination with or without adjuvant (AdEtOH). Different concentrations of antigen were used. Each spot corresponds to the activation of a T-cell.

In the case of vaccination with peptide antigens or with larger antigenic polypeptide fragments, one of the main problems is the efficient transfer of the antigens to the MHC molecules. The efficiency is additionally reduced by the proteases present in the serum, which degrade the free antigens within a relatively short time. An acceleration of the antigen transfer, as effected by the methods according to the invention, accordingly has a positive effect in the vaccination in two respects on the enhancement of the immune response. This can be achieved, for example, by adding catalysts such as, for example, AdEtOH as additive or adjuvant to the vaccine.

FIG. 16 shows the use of the MHC binding assay for identifying immunotoxic compounds. To this end, two compounds which can be used in methods according to the invention were employed (3-N-phenylaminopropanediol, PAP, (III 36) (o) and sulfamethoxazole (III 37) (∇) in comparison with the pCP known from the prior art). This assay was carried in vitro. As will be seen from the figure, both compounds, in contrast to most other compounds, are able to accelerate the loading of MHC molecules with peptide ligands. Although the activity is markedly lower than that of pCP, it is still clearly detectable with the assay shown here. It is thereby demonstrated that the use of methods according to the invention, such as, for example, the MHC loading assay, offers the possibility of identifying at least some of these compounds in vitro by means of their catalytic activity. In particular, compounds whose immunotoxic activity is presumably attributable to a direct influencing of the load state of the MHC can thereby be traced in corresponding screening methods according to the invention. This is illustrated here by means of two examples, 3-N-phenylaminopropanediol (PAP) and sulfamethoxazole (SMX). PAP is an aniline derivative which is associated with the formation of an autoimmune syndrome (toxic oil syndrome) (Gelpi E., et al., Environ Health Perspect. 110:457), while SMX can trigger an allergy by binding directly to the MHC class II molecule (Burkhart C., et al. Clin Exp Allergy. 32:1635).

### EXAMPLES

The following non-limiting examples describe the present invention and do not limit the invention in any way. The examples provide the person skilled in the art with guidance for the use of the compounds and methods of the invention. In any case, other compounds within the invention can be replaced by those of the example compound indicated hereinbelow with similar results. The experienced practitioner will understand that the examples constitute guidance and can be varied on the basis of the different compounds to be used.

### 1. In Vitro Methods

1.1. Analysis of Synthetic Catalysts In Vitro

1.1.1 Recombinant Soluble HLA-DR Complexes

Soluble MHC class II molecules HLA-DR1 (DRA1*0101, DRB1*0101), HLA-DR2 (DRA1*0101, DRB1*1501) and HLA-DR4 (DRA1*0101, DRB1*0401) were produced in S2 insect cells. These had been stably transfixed with vectors that code for shortened α- and □-chains without the cytoplasmatic and transmembrane portion of the MHC molecule following a metallothionine promoter. The HLA-DR-producing cells were cultivated in a shaking culture at 26° C. in serum-free HyQ-SFX insect medium (HyClone). Induction of HLA-DR production was carried out at a cell density of 6-8×10⁶/ml by addition of 1 mM CuSO₄. Production was then carried out for a period of 5 days.

1.2.2 Purification of Soluble HLA-DR Complexes from Cell Culture Media

The cell supernatants of the HLA-DR-producing cells were passed in a cycle for several days over three series-connected 25 ml affinity columns by means of a peristaltic pump with a flow rate of 1.6 ml/minute. The first column contained pure protein A agarose and the second column contained protein G agarose. The third column contained protein A agarose to which LB3.1 anti-HLA-DR antibody had been coupled. All three columns were then first washed with 500 ml of PBS-0.02% sodium azide and the antibody-coupled column was then washed on its own with 200 ml. This column was then equilibrated in a BiologicHR Chromatography System with 50 ml of 10 mM NaH₂PO₄, and bound HLA-DR was eluted with 50 mM CAPS (pH 11.5). The elution was monitored by measuring the absorption at 280 nm, and the eluate was captured during a peak in 1 ml of 600 mM NaH₂PO₄. Finally, the column was neutralised by washing with 50 ml of 300 mM NaH₂PO₄. The protein concentration in the captured eluate was determined by means of protein determination according to Bradford.

1.2.3 MHC Class II Loading Experiments

In order to monitor catalysis of the loading of HLA-DR molecules by compounds of formulae I, IA, II, III and IV1 to IV3, empty, soluble DR molecules were incubated with biotinylated, high-affinity peptide, and these were then detected in a sandwich ELISA for the MHC complexes.

To this end, about 0.5 μg of HLA-DR on ice was mixed with 1 μg of high-affinity, biotinylated peptide (HA-biot. (biot. HA306-318) in HLA-DR1&DR4 loading experiments and MBP-biot. (MBP86-100) in HLA-DR2 loading experiments) and with the concentration of catalyst substance indicated in each test. All the batches were then adjusted to a final volume of 10 μl with 2% BSA-PO₄³⁻ buffer (in the case of HLA-DR1 only with PO₄³⁻ buffer). The reaction was started by incubation at 37° C. in a thermocycler. After 40 minutes, the incubation was terminated and the loading reaction was stopped by storage on ice and addition of 50 μl of ice-cold 1% BSA-PBS.

The detection of bound peptide was carried out by means of a sandwich ELISA. To this end, 96-well Maxisorp plates were coated overnight with 80 μl/well of α-HLA-DR antibody (clone L243 (1 mg/ml) diluted 1:500 in 100 mM NaHCO₃ solution) and then blocked at 37° C. for at least one hour with 200 μl/well of 2% BSA-PBS. Between these steps, the plate was washed twice with PBS-0.05% Tween with the aid of a PW plate-washing device (Tecan Industries) and, before filling with fresh solution, all remaining liquid residues were removed by tapping on Scott®Natura paper towels (Hakle-Kimberly Deutschland GmbH). After blocking of non-specific binding sites, the plate was washed three times and filled with 50 μl/well of 1% BSA-PBS. 2×25 μl per reaction batch were transferred to the plate as double values and were carefully mixed thereon (total volume per well: 75 μl). From each filled series, a further 25 μl/well were then again titrated on the plate in a series of two-stage dilutions (final volume per well: 50 μl). The plates so filled were then stored for 2 hours at 4° C. under stationary conditions. Each plate was then washed six times as described above and filled with 80 μl/well of Eu³⁺-streptavidin staining solution (europium-streptavidin stock solution diluted 1:10,000 with 1% BSA-PBS) and incubated for 30 minutes at room temperature (RT). After washing for a further eight times, 100 μl/well of fluorescence-activated solution (enhancer) were then added and the signal was measured by means of a Victor²-multilabelcounter (Wallac Oy) at an excitation wavelength of 340 nm and an emission wavelength of 615 nm in time-resolving mode.

1.2.4 Analysis of the Substance Library

The compounds of formulae I, IA, II, III and IV1 to IV3 to be tested were supplied in 384-well plates in the form of a 160 mM starting library, dissolved in DMSO, from which an aliquot library had to be prepared. By manually removing 2 μl/well and diluting with 38 μl/well of DMSO, a 8 mM stock library was prepared, which was used for all further tests. In order to analyse the compounds of formulae I, IA, II, III and IV1 to IV3 that were present for their effect on MHC class II molecules, a high-throughput method based on a HLA-DR1 loading test was used. To this end, two 384-well Nunc-Maxisorp plates (ELISA plates) per library plate were coated overnight at 4° C. with 60 μl/well of monoclonal anti-HLA-DR antibody (L243 stock solution (1 mg/ml) diluted 1:500 in 100 mM NaHCO₃). The plates were then washed three times by immersion in PBS-0.05% Tween solution and removal of air bubbles by tapping the plate against the wall of the washing vessel. Remaining washing solution was removed by beating the plates on paper towels, and 80 μl/well of 2% BSA-PBS were introduced. Non-specific binding was to be prevented by incubation at 37° C. for at least one hour. During that time, a 384-well NUNC polystyrene plate (analysis plate) per library plate was filled with 22 μl/well of DR1 analysis solution (about 12 μg/ml of HLA-DR1 in PO₄³⁻ buffer) and stored under cool conditions. Then, with the aid of a 384-well pipetting robot, 1 μl/well was transferred from the library plate to the analysis plate (final volume 23 μl/well) and the tips of the robot were washed. This was effected by drawing up and ejecting 20 μl of solution five times in succession in a DMSO, a water and an ultrasonic continuous-flow bath filled with distilled water. After ejection of any liquid that had remained in the tips, 2 μl/well were transferred from a 384-well plate filled with HA-biot. solution (1 mg/ml HA-biot. dissolved in PBS) to the analysis plate and were there mixed carefully with the HLA-DR mixture containing compounds of formulae I, IA, II, III and IV1 to IV3 by adding and removing 15 μl/well by means of a pipette (final volume 25 μl/well). The analysis plate so filled was then incubated for 40 minutes at 37° C. and the tips of the robot were washed as described above (instead of DMSO, a further water bath was used here!). Shortly before the end of this period, the two ELISA plates were washed five times as described, remaining washing solution was removed, and 30 μl/well of 1% BSA-PBS solution were introduced by means of the robot. After the 40-minute incubation, 40 μl/well of 1% BSA-PBS were additionally introduced into the analysis plate by means of the pipetting robot and were mixed with the reaction solution by introducing and removing 30 μl/well five times by means of a pipette (final volume in the analysis plate about 65 μl/well). From this BSA-PBS reaction mixture, the robot then transferred 2×30 μl/well into the two ELISA plates and mixed them with the BSA-PBS solution already present by again introducing and removing 30 μl/well by means of a pipette (final volume in the ELISA plates 60 μl/well). The ELISA plates so filled were then stored for two hours at 4° C., and the tips of the robot were washed in three water baths by drawing up and ejecting 40 μl ten times. The two ELISA plates were then washed six times in the manner already described and filled manually with 60 μl/well of Eu³⁺ staining solution. In order to ensure adequate labelling of the bound biotinylated peptide, the plates were then stored under stationary conditions for at least 30 minutes at RT. Excess staining solution was then removed by washing eight times with PBS-0.05% Tween, and 80 μl/well of fluorescence-activated solution (enhancer) were added manually by means of a pipette. Detection of the signal was then carried out as described in the preceding section.

1.2.5 Validation of Catalysts Identified in the Library

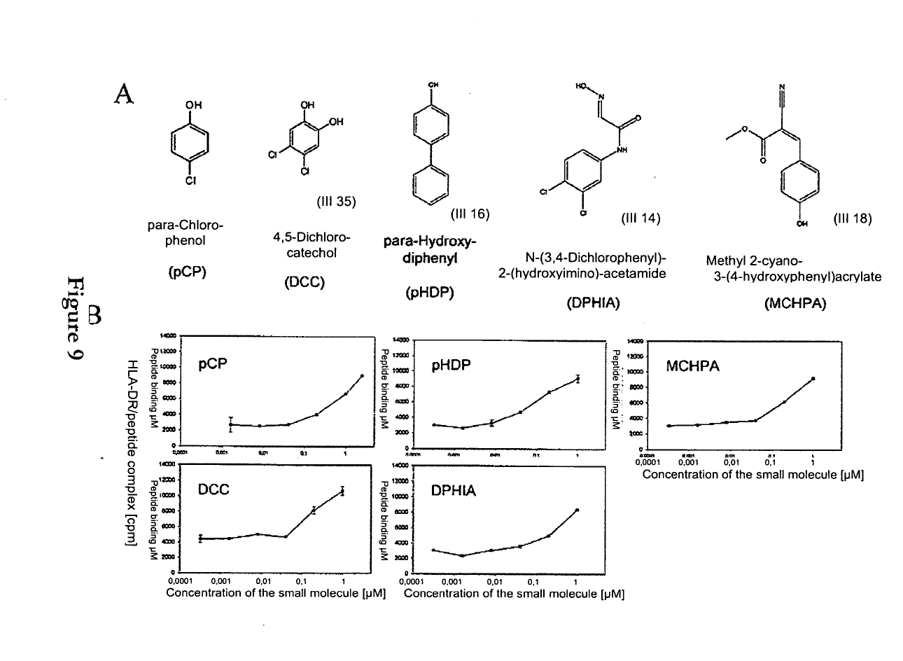

1.2 μl of the respective substance from the library plate were mixed with 4.8 μl of PO₄³⁻ buffer, and 1 μl thereof was transferred to a fresh Eppendorf vessel and mixed therein with 5 μl of PO₄³⁻ buffer again. In a further dilution step, a further 1 μl was then removed from this second dilution stage and mixed with 7 μl of PO₄³⁻ buffer. 0.5 μg of HLA-DR1 and 1 μg of HA-biot. were then added to all three dilution stages on ice, and the final volume was adjusted to 10 μl with PO₄³⁻ buffer. As a result, a 1:10, a 1:50 and a 1:300 dilution of the substance from the library were obtained. As control, the procedure described for the library substances was carried out with DMSO and a 10 mM stock solution on 1,2-dichloro-4,5-dihydroxybenzene/dichlorocatechol (see e.g. FIG. 9). Further analysis was carried out as described under MHC class II loading experiments.

1.3 Study of Natural Potential Catalyst Sources

1.3.1 Obtention and Extraction of Human Plasma

Blood samples were taken from patients and introduced into test tubes coated with HEPES. The freshly taken blood was immediately centrifuged for 15 minutes at 1500 rpm and the plasma was removed.

Extraction of the plasma was carried out substantially as described by Gamache et al. (1998, Proc Soc Exp Biol Med 217:274-280). In detail, 1.6 ml of EtOH were added to 0.4 ml of human plasma, and mixing was carried out for 5 minutes in a MS2 Minishaker by vortexing at the highest level. Precipitated protein was separated off by centrifugation for 10 minutes at 13,200 g in a 5804 R centrifuge from Eppendorf, and the supernatant was taken up in a fresh Eppendorf vessel. The extract so formed was then concentrated in a SPD111V SpeedVac under reduced pressure and at about 40° C. for 2 hours, to a final volume of about 100 μl. 50 μl of this concentrate were then used directly in the HPLC analysis.

1.3.2 HPLC Analysis

Analytical high-pressure liquid chromatography (HPLC) was carried out in a Pharmacia Biotech Smart™System equipped with a C2/C18 SC2.1/10 RP (reverse phase) chromatography column. Substances present were detected by means of a UV detector which at the same time recorded the absorption at 214 nm, 260 nm and 280 nm at RT. The mobile phase consisted of 0.096% trifluoroacetic acid (TFA) in H₂O or of 0.104% TFA in acetonitrile. Eluates were collected by time-dependent fractionation with a volume of 1 ml. The solvent was then removed completely under reduced pressure at 40° C. in a SPD111V SpeedVac (Savant Instruments Inc.) and the pellet was resuspended in 5 μl of DMSO. Further investigation of the fractions was carried out by HLA-DR1 loading experiments.

1.4 Toxicity Analyses

In order to determine the toxicity of the found substances, from 50,000 to 100,000 cells (L57.23) in 100 μl of DMEM were mixed with 50 μl of the catalysts in the indicated concentrations (dissolved in DMEM at a maximum concentration of 3% DMSO) and incubated for four hours at 37° C. and 10% CO₂ in a humid incubator. As control, only 50 μl of DMEM were added to cells. The cells were then centrifuged off for 5 minutes at 1300 rpm, and the supernatant medium was discarded carefully. The cell pellets were then detached by brief vortexing of the entire plate at a low level, resuspended with 100 μl/well of 5% FCS-PBS and centrifuged again for 5 minutes at 1300 rpm. The supernatant was then discarded again and the cell pellets were dissolved in 100 μl/well of propidium iodide staining solution (25 pg/ml). The cell suspension was then transferred to 5 ml Falcon® round-bottomed test tubes, which had previously been filled with 300 μl of 5% FCS-PBS, and then analysed using a BD FACSCalibur™System continuous-flow cytometer.

1.5 Loading of MHC Class II Molecules on the Surface of Cells

The catalytic properties of found substances on the loading of MHC class II molecules located on cells was determined in cell loading experiments. To this end, from 50,000 to 100,000 cells in 50 μl of DMEM were mixed with 50 μl of 24 μM HA-biot. dissolved in DMEM and 50 μl of catalyst solution (different concentrations dissolved in DMEM with a maximum DMSO content of 3%) and incubated for 4 hours at 37° C. and 10% CO₂. As controls, only 50 μl of peptide solution and 50 μl of DMEM were added to cells in several batches. At the end of the incubation time, the cells were centrifuged off for 5 minutes at 1300 rpm, and supernatant medium was removed. The cell pellets were then detached by brief vortexing of the plate, resuspended in 100 μl/well of 5% FCS-PBS and centrifuged again for 5 minutes at 1300 rpm. After removal of the supernatant, the cell pellets were again detached and taken up in 50 μl/well of streptavidin-phycoerythrin solution (SA-PE stock solution (1 mg/ml) diluted 1:200 with 2% FCS-PBS). Staining was carried out for 30 minutes at 4° C. with the exclusion of light, in order to avoid excessive decomposition of the fluorescent dyes. The stained cells were then centrifuged off again as already described, washed with 2% FCS-PBS and taken up in 100 μl/well of 2% FCS-PBS and transferred to 5 ml Falcon® round-bottomed test tubes, which had previously been filled with 300 μl of 2% FCS-PBS. The cells were then analysed using a continuous-flow cytometer. The expression of MHC molecules on the surface of the cells was checked in control batches by staining with α-HLA-DR antibody (labelled with PE, stock solution (1 mg/ml) diluted 1:75 with 2% FCS-PBS) or as isotype control with IgG2a mouse antibody (labelled with PE, stock solution (1 mg/ml) diluted 1:75 with 2% FCS-PBS) instead of with SA-PE (streptavidin-PE).

1.6 Continuous-Flow Cytometry

Analyses by continuous-flow cytometry were carried out on a BD FACSCalibur™System from Becton Dickinson. For staining there were used phycoerythrin-labelled streptavidin (CALTAG Laboratories), α-HLA-DR antibody and IgG2a mouse antibody (BD-Biosciences), both labelled with phycoerythrin as well as propidium iodide (Sigma-Aldrich-GmbH). All other materials necessary for the measurement were obtained from BD Biosciences, Bedford, USA.

1.7 Analysis of the Substance Library

For the identification of novel substances or classes of substance that are capable of influencing the interactions between MHC class II molecules and their ligands, similarly to HLA-DM but at physiological pH values, a 20,000-component small molecule library (Chemical Diversity Labs, Inc.) was analysed, by means of a high-throughput analysis method, for compounds that catalyse the loading of empty, soluble HLA-DR1 complexes with high efficiency. As reference substance there was used 1,2-dichloro-4,5-dihydroxy-benzene/dichlorocatechol (see e.g. FIG. 9), a substance that still exhibited activity at low concentrations.

1.8.1 High-Throughput Analysis of the 20,000-Component Library

Analysis of the library present in 384-well plates was carried out by means of a Quadra-384 pipetting robot. Because all the wells of the library plates were filled with substance, an extra plate was prepared as control, which plate was filled predominantly with DMSO and, in some wells, with pCP and 1,2-dichloro-4,5-dihydroxybenzene/dichlorocatechol (see e.g. FIG. 9) in different concentrations. This control plate was then included in the analysis for active substances in parallel with the actual library plates. Analysis of the library is based on a high-throughput analysis method described above. Most substances are present in a relatively narrow range, while individual substances extend beyond this background loading. Comparison with the values achieved for the control plate provided a threshold value of 15,000 counted events, because that value is achieved by 1,2-dichloro-4,5-dihydroxybenzene/dichlorocatechol (see e.g. FIG. 9) at a final concentration of 0.4 mM (which corresponds approximately to the concentration of the compounds used from the library). All substances that were able to catalyse a higher load were classified as more catalytically active than 1,2-dichloro-4,5-dihydroxybenzene/dichlorocatechol (see e.g. FIG. 9) and were investigated further in subsequent experiments.

1.8.2 Validation of the Compounds Identified in the Library

The results obtained from the analysis of the library were first confirmed for all substances evaluated as being catalytically active. In order to expand the results achieved with this experiment, the catalysts were thereby titrated and used in the dilution stages 1:10, 1:50 and 1:300. 1,2-Dichloro-4,5-dihydroxybenzene/dichlorocatechol (see e.g FIG. 9) and DMSO served as reference. The standard used was that all compounds are evaluated as positive that delivered a substantially stronger signal than 1,2-dichloro-4,5-dihydroxy-benzene/dichlorocatechol (see e.g. FIG. 9) in the first stage and then declined, or that catalysed a load that was at least equally as high or higher over the course of the three dilutions.

The investigations found substances whose catalytic activity was classified either as equally as high as that of 1,2-dichloro-4,5-dihydroxybenzene/dichlorocatechol (see e.g. FIG. 9) or even as higher. The structural formulae of the compounds so found by way of example are shown in Table 5.

TABLE 5
Structural formulae of catalysts identified by way of example
from the library. The graphics were prepared by means of the program
ISIS ™/Draw2.4 (MDL Information Systems, Inc., USA). The indicated
IUPAC names were determined by the accompanying
PlugIn AutoNom Standard.

| | |
|---|---|
| | I1 |
| 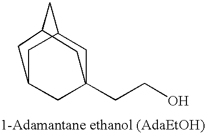 | |
| | I2 |
| 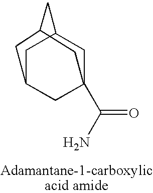 | |
| | I3 |
| 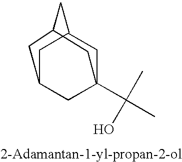 | |
| | I4 |
| 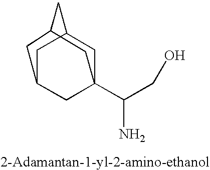 | |
| | I5 |
| 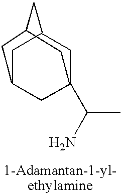 | |
| | I6 |
| 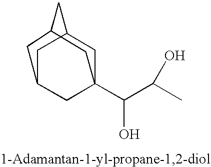 | |
| | I7 |
| 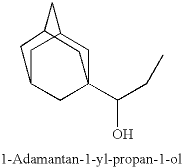 | |
| | I8 |
| 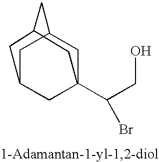 | |
| | I9 |
| 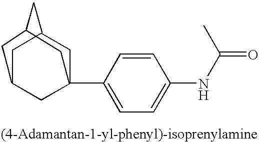 | |
| | I10 |
| 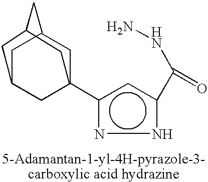 | |
| | I11 |
| 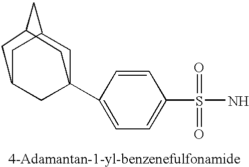 | |
| | I12 |
| 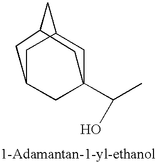 | |
| | I13 |
| 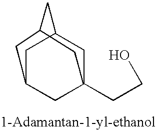 | |
| | I14 |
| 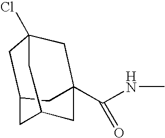 | |
| | I15 |
| 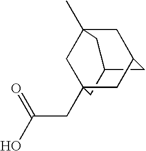 | |
| | I16 |
| 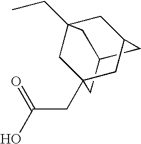 | |
| | II1 |
| 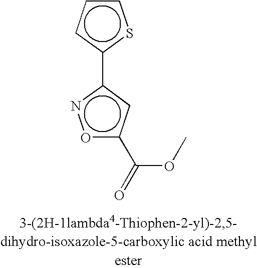 | |
| | II2 |
| 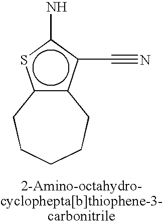 | |
| | II3 |
| 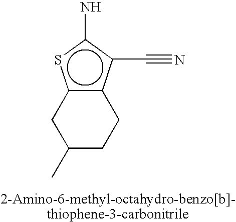 | |
| | II4 |
| 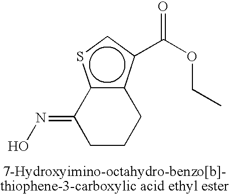 | |

| | |
|---|---|
| | III1 |
| 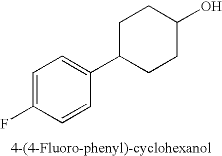 | |
| | III2 |
| 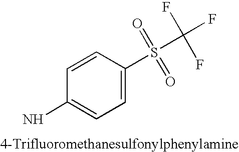 | |
| | III3 |
| 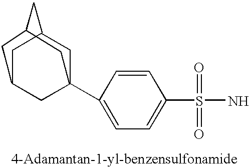 | |
| | III4 |
| 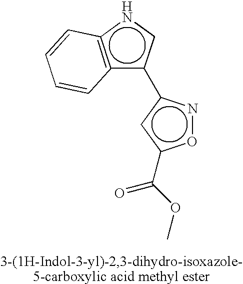 | |
| | III5 |
| 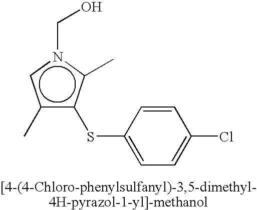 | |
| | III6 |
| 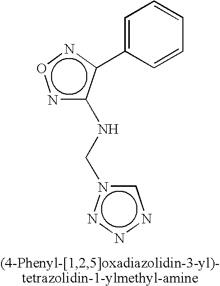 | |
| | III7 |
| 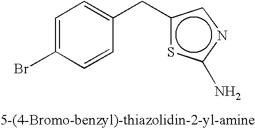 | |
| | III8 |
| 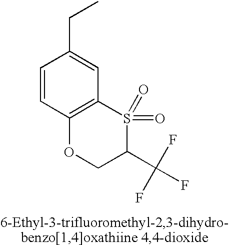 | |
| | III9 |
| 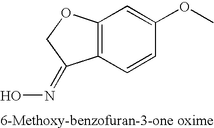 | |
| | III10 |
| 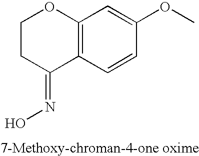 | |
| | II11 |
| 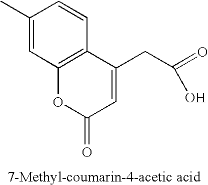 | |
| | II12 |
| 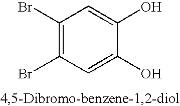 | |
| | III13 |
| 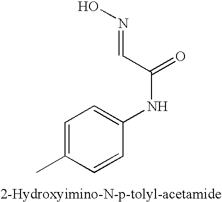 | |
| | III14 |
| 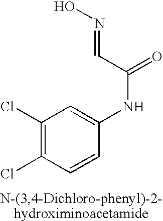 | |
| | III15 |
| 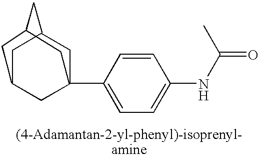 | |
| | III16 |
| 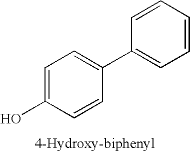 | |
| | III17 |
| 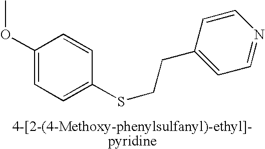 | |
| | III18 |
| 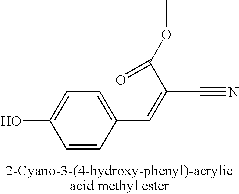 | |
| | III19 |
| 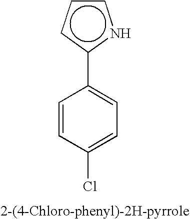 | |
| | III20 |
| 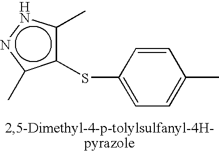 | |
| | III21 |
| 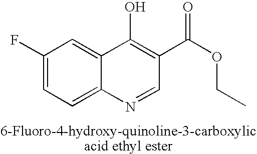 | |
| | III22 |
| 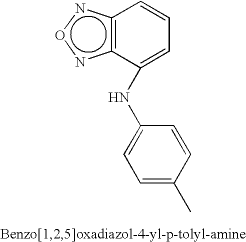 | |
| | III23 |
| 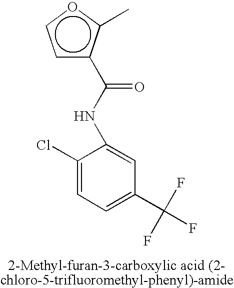 | |
| | III24 |
| 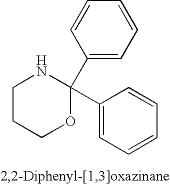 | |
| | III25 |
| 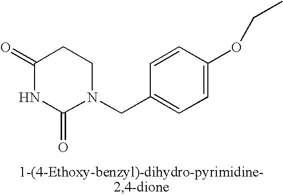 | |
| | III26 |
| 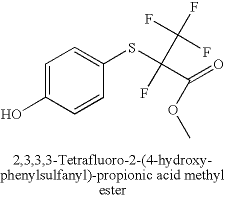 | |
| | III27 |
| 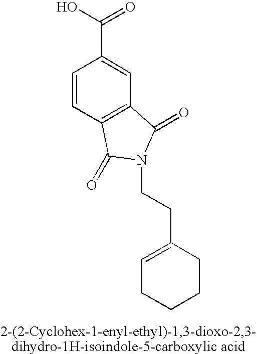 | |
| | III28 |
| 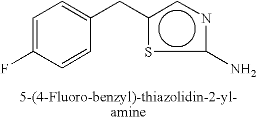 | |
| | III29 |
| 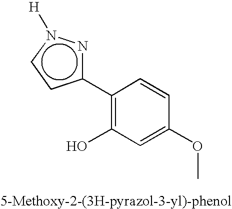 | |
| | III30 |
| 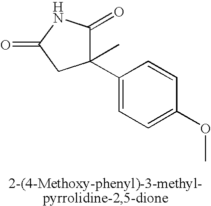 | |
| | III31 |
| 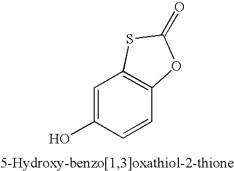 | |
| | III32 |
| 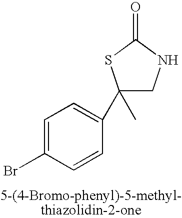 | |
| | III33 |
| 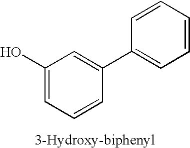 | |
| | III34 |
| 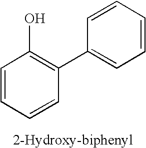 | |
| | IV1 |
| 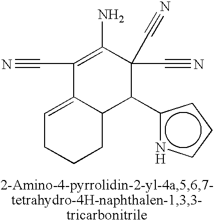 | |
| | IV2 |
| 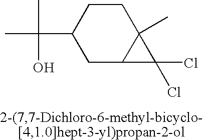 | |
| | IV3 |
| 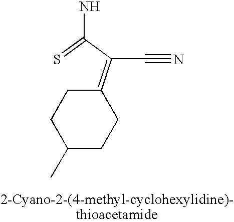 | |

In order to check the system of analysis, the particular compounds of formulae I, IA, II, III and IV1 to IV3 were tested in a HLA-DR1 loading batch for their activity. The compounds homologous to adamantane ethanol (I1), whose position in the library could also be determined, are shown in Table 6. As will be seen, differences in activity occur. The loading of the substituents evidently plays a part above all, because the acid-amide-substituted form of adamantane (I2) again exhibits increased catalytic activity. The slightly reduced activity of compound I3 might be attributable to steric effects. Therefore, the adamantyl structure is evidently critical for the catalytic activity, while the side chain can be attributed with modulating properties.

TABLE 6
Analysis of homologous compounds using the example of adamantane
ethanol. Structural formulae of the catalyst adamantane ethanol identified
by the analysis and of six homologous compounds contained
in the library.

| | |
|---|---|
| | I1 |
| 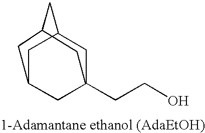 | |
| | I2 |
| 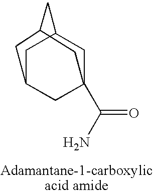 | |
| | I3 |
| 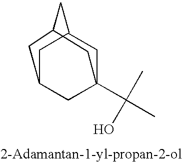 | |
| | I4 |
| 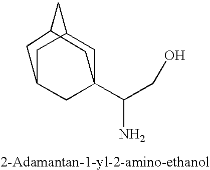 | |
| | I5 |
| 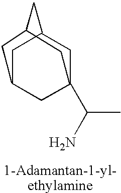 | |
| | I6 |
| 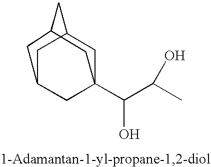 | |
| | I7 |
| 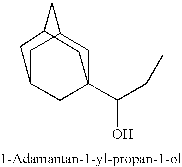 | |

1.8.3 Additive Effects

The observation that the identified compounds of formulae I, IA, II, III or IV1 to IV3 have different effects on the individual allelic variants might lead to the conclusion that the tested compounds attack different catalytic centres on the MHC complex. The great structural diversity of the found substances could also support this assumption. Should this actually be the case, then the activities of the individual catalysts might be added together or even potentiated when the catalysts are incubated together. The following experiment was therefore carried out. One catalyst in a fixed concentration of 0.05 mM was introduced into each of a number of batches for loading HLA-DR1 with HA-biot. The same concentration of a second catalyst was then added to each batch. As controls, 1×1% DMSO was added and, as negative control, a reaction mixture was prepared with 2×1% DMSO. The reaction mixtures were then incubated for 40 minutes as already described and the load was measured in a sandwich ELISA.

The result obtained in this test is a relative increase in the load by about 10% (+DMSO). If the amount of 2-HBP is doubled (+2-HBP), this value increases as expected because, as has already been shown, the effect is concentration-dependent. The addition of 0.05 mM AdaEtOH increases the load to almost 50%, however, and accordingly indicates a drastic increase in the catalytic effect.

### 2. Tests at Cell Level

2.1 Analysis of the Activity on MHC-Expressing Cells

In order to demonstrate a possible physiological relevance of such compounds, the activity of the previously identified compounds on cells was analysed. To this end, the influence of AdaEtOH, DPHIA and 4-HBP on MHC class II-expressing cell lines has been studied hereinbelow.

2.1.1 Toxicity

In addition to the requirement that the substances identified in the library should exhibit increased catalytic activity, a reduced or equivalent toxicity as compared with pCP was required as a second criterion for assessment. Although AdaEtOH showed toxic activity, it exhibits substantially greater catalytic activity in the ELISA. This means that it might possibly be used in concentrations that are reduced by a factor sufficient to rule out toxic effects. The use of concentrations of AdaEtOH in the lower μM range, as could be detected in the ELISA, would offer suitable requirements therefor. The same is also true of DPHIA, which exhibits even lower toxic effects than AdaEtOH or 4-HBP and delivered similarly good results as AdaEtOH in the ELISA.

2.1.2 Loading of MHC Complexes on the Membrane

On the basis of the results obtained from the preceding experiments, cell loading experiments with L57.23 (HLA-DR1, DRB1*0101) and L243.6 cells (HLA-DR4, DRB1*0401) were prepared in the following. The loading of the cells with 8 μM HA-biot. was thereby catalysed by AdaEtOH, DPHIA and 1,2-dichloro-4,5-dihydroxybenzene/dichlorocatechol (see e.g. FIG. 9) in the concentrations 0.1 mM, 0.05 mM and 0.025 mM. Loading with pCP in the concentrations 1 mM, 0.5 mM and 0.25 mM was used as control. In order to adjust the system, the expression of the MHC complexes on the cell surface was again used. The result of the loadings for pCP and AdaEtOH shows that AdaEtOH catalyses an almost equally high loading of the membrane-located HLA-DR1 complexes compared with pCP at ten times lower concentrations. The loading of HLA-DR4 complexes even gives an increased loading compared to pCP with a tenth of the concentration. Accordingly, a drastic improvement in the catalytic activity as compared with pCP could be achieved both with soluble MHC molecules and in MHC-expressing cells.

### 3. Comparison Tests

3.1 Catalysis of the Loading of Soluble HLR-DR1 Molecules by Adamantyl Compounds

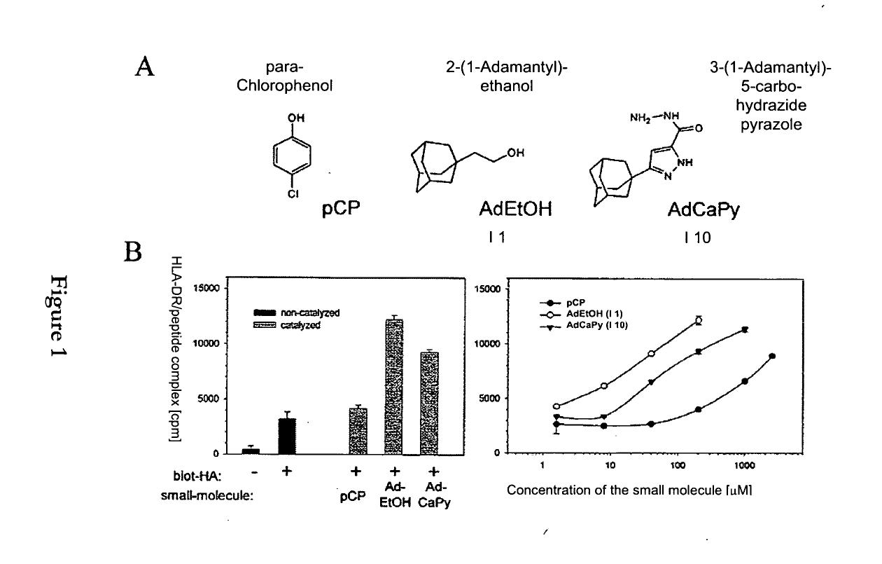

In the present experiment, p-chlorophenol (pCP), 2-(1-adamantyl)-ethanol (AdEtOH) and 3-(1-adamantyl)-5-carbohydrazide pyrazole (AdCaPy) were used to catalyse the loading of empty soluble HLA-DR1 molecules with the HA306-318 peptide. The reactions were carried out in the presence of 250 μM pCP, AdEtOH or AdCaPy. The resulting load was then determined in an ELISA using biotinylated peptide (see FIG. 1). As is clear from FIG. 1, all three catalysts are capable in principle of accelerating the reaction. Compared with the pCP curve, however, the corresponding curves of the adamantyl compounds are displaced to the left by more than a power of ten, i.e. they are at least 10 times more active than pCP.

3.2 Catalysis of the Loading of HLA-DR Molecules on the Cell Surface

In the present test, the catalysis of the loading of HLA-DR molecules on the cell surface was studied, and dose-activity curves for the loading of fibroblast cells with the HA306-381 peptide were drawn up. To this end, the cells were incubated for 4 hours with the peptide and with titrated amounts of pCP, AdEtOH or AdCaPy at 37° C. in culture medium. By using a biotinylated peptide, it was then possible, after staining of the cells with fluorescent streptavidin, to detect the amount of bound peptide on L57.23 and on L243.6, which express HLA-DR1 (DRB1*0101) and HLA-DR4 (DRB1*0401), respectively, two MHC class II molecules which are able to present the HLA306-318 peptide. The absence of any staining of the L929 cells indicates that all the catalysts selectively enhance the binding to the surface MHC molecules. A comparison of the dose-activity curves confirms the result with the soluble MHC molecules (see also FIG. 1). Here too, the tested adamantyl compounds are found to be substantially more active than pCP and accelerate the loading of the cells even at a concentration markedly below a tenth of the corresponding pCP concentration.

3.3 Enhancement of the T-Cell Response by Catalysis of the Loading of HLA-Expressing Cells

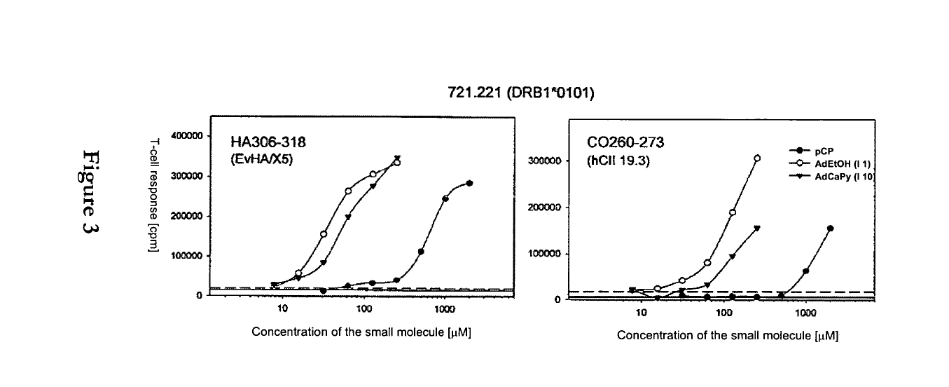

In the present comparison test, the enhancement of the T-cell response by catalysis of the loading of HLA-expressing cells was investigated. To this end, the dose-activity curves of the immune response of two different HLA-DR1-restricted T-cells were produced and, to this end, HLA-DR-expressing 721.221 cells were incubated for 4 hours as described in Comparison Test 3.2 with titrated amounts of pCP, AdEtOH or AdCaPy. Either HA306-318 (see also FIG. 3, left-hand panel) or CO260-273 (see also FIG. 3, right-hand panel) was used as peptide antigen. After the loading, the cells were washed and used to stimulate EvHA/X5 and hCII19.3 cells, respectively. In both cases, the immune response of the T-cells is markedly enhanced by the compounds AdEtOH or AdCaPy used in the method according to the invention. A comparison of the dose-activities shows that the adamantyl compounds are also about 10 to 1000 times more effective than pCP in respect of the T-cell response.

3.4 Allele-Specific Activity of the Catalysis by Adamantyl Compounds

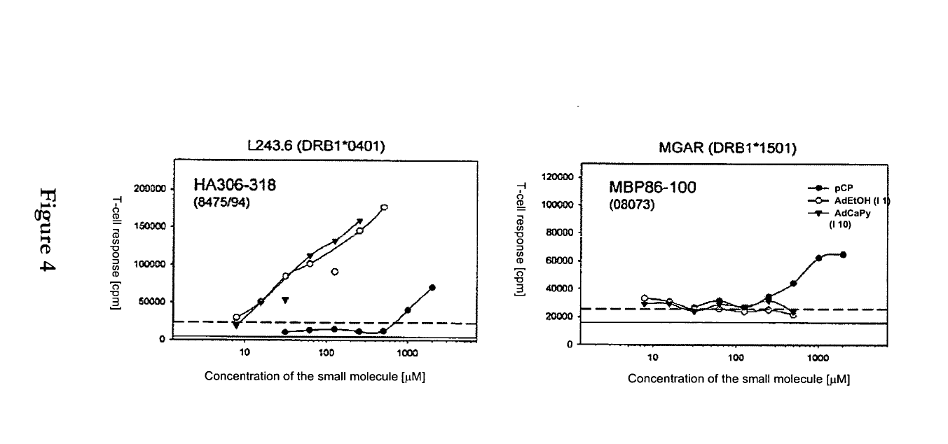

In order to investigate the allele-specific activity of the catalysis by adamantyl compounds, HLA-DR4 (DRB1*0401)- and HLADR2 (DRB1*1501)-expressing cells were loaded with HA306-318 or MPB86-100 in the presence of the catalysts and then used in the T-cell assay. For evaluation, dose-activity curves of a HLA-DR4-restricted (FIG. 4, left-hand panel) and a HLA-DR2-restricted T-cell response (FIG. 4, right-hand panel) were prepared. While the dose-activity curves of the HLA-DR4-restricted 8475/94 T-cells correspond to those of the HL-DR1-restricted T-cells described hereinbefore in Comparison Experiment 3.3, a marked difference is to be seen in the case of the curves of the HLA-DR2-restricted 08073 T-cells. In contrast to pCP, which can have an enhancing effect on all the HLA-DR molecules investigated, the adamantyl compounds are evidently allele-specific.

3.5 Investigation of the Causes of Allele-Specific Activity

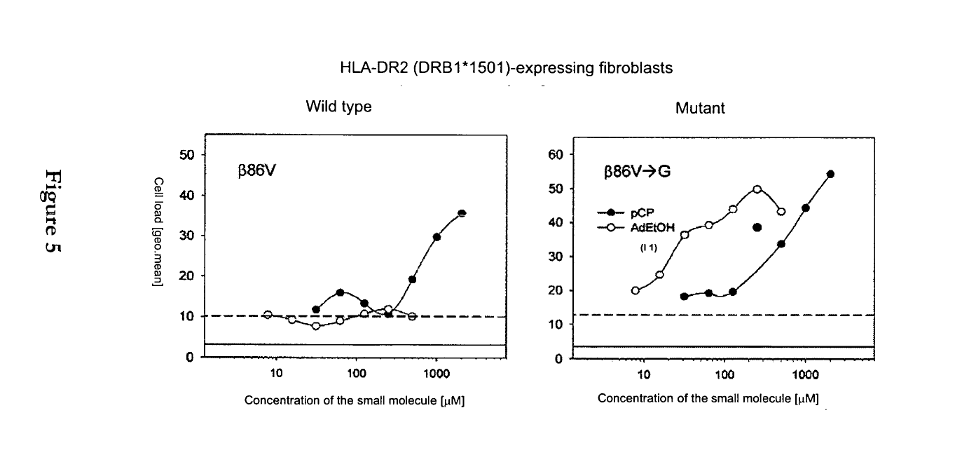

The allele-specific activity of the adamantyl compounds is evidently based on the interaction with the “deep” P1 pocket. Because the allele-specific activity of the adamantyl compounds evidently correlates with a dimorphism at position 86 of the HLA-DR β-chain, this assumption was investigated by carrying out a corresponding mutation in the HLA-DR2 (DRB1*1501) molecule, followed by expression in fibroblast cells. The results are shown in FIG. 5. FIG. 5 shows a comparison of the dose-activity curves of the catalysis of the binding of MBP86-100 to the HLA-DR2 wild-type molecule (see Figure, left-hand fig.) and to the mutated HLA-DR2 molecule, in which the valine residue at position 86 has been replaced by glycine (see Figure, right-hand fig.). While adamantane ethanol is unable to effect catalysis on the unmutated HLA-DR2 molecule of the fibroblast cells, as on the MGAR cells described in FIG. 4, the V→G substitution has the effect of making the mutated HLA-DR2 molecule receptive to the adamantyl-mediated catalysis again. Because it is known that the glycine/valine dimorphism determines the depth of the binding pocket P1, where 86G correlates with a deep pocket, adamantyl compounds accordingly evidently mediate their catalytic activity by their binding to a deep P1 pocket.

3.6 Catalysis of a HLA-DR1 (DRB1*O101)-Restricted T-Cell Response

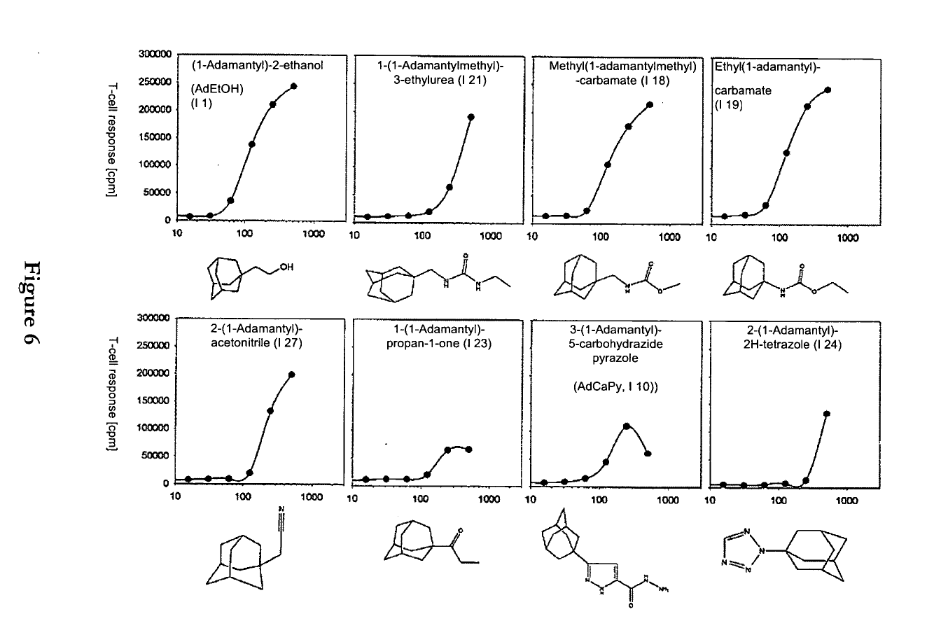

In order to investigate the catalysis of a HLA-DR1 (DRB1*0101)-restricted T-cell response by various catalytically active adamantyl compounds, dose-activity curves of the catalysis of a HLA-DR1 (DRB1*0101)-restricted T-cell response were prepared. The experiment was carried out analogously to Comparison Experiment 3.3, the peptide antigen HA306-318 being loaded onto 721.221 cells in order thereby to stimulate EvHA/X5 T-cells. The results are shown in FIG. 6. The dose-activity curves show that all the listed adamantyl compounds possess catalytic activity. The in some cases very different chemical nature of the side chains suggests that the activity is mediated substantially by the adamantyl grouping. Because the position of the dose-activity curves is evidently also determined by the side chain, a modulating activity can be attributed thereto.

3.7 Loading of MHC Class II Tetramers by Means of Adamantyl Compounds

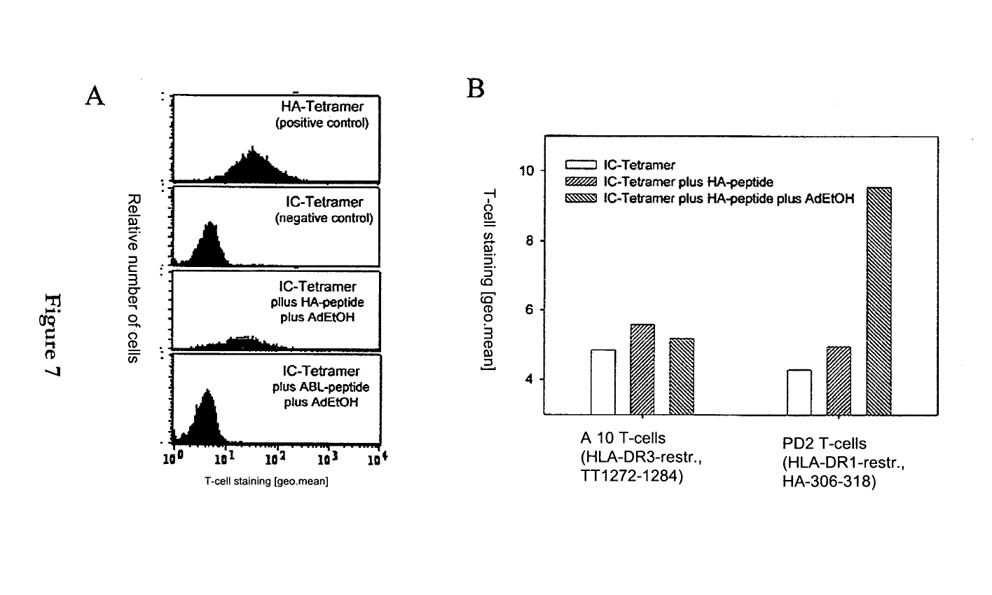

In the present comparison test, the loading of MHC class II tetramers by means of adamantyl compounds was investigated in detail. To this end, the following tetramers were used: HA tetramers, HLA-DR1 tetramers, which were obtained from the manufacturer (Proimmune Ltd.) already loaded with the T-cell antigen HA306-318, and IC tetramers, which carry the non-relevant peptide IC 106-120.
A) In order to demonstrate that adamantyl compounds are able to catalyse the loading of tetramers, IC tetramers were incubated overnight with AdEtOH and HA306-318. Loading with a non-relevant peptide (ABL) was additionally carried out as control. After staining of the cells with the complexes, analysis by continuous-flow cytometry showed that the signal delivered by the PD2 T-cells, which were stained by means of adamantylethanol-loaded tetramers, was of a similar height to that delivered by the pre-prepared HA tetramers.
B) In order to ensure that the replacement of the IC peptide by the HA peptide is actually attributable to the catalysis by adamantylethanol, the ligand replacement was carried out in a second experiment in the presence and also in the absence of adamantylethanol. The complexes formed thereby, like the untreated IC tetramer, were then used again to stain the PD2 cells, and also as negative control to stain HLA-DRw52a (DRB3*0101)-restricted TT1272-1284-specific A10 T-cells. As will be seen in the bar diagram in FIG. 7, in this experiment too the HA306-318-specific PD2 cells are stained with those tetramers catalysed by adamantylethanol. By contrast, no appreciable staining is observed with the complexes in which loading was attempted without catalyst. The complexes formed by means of adamantylethanol additionally exhibit the required antigen specificity, because A 10 T-cells, in contrast to PD2, are not stained.

The results from Comparison Test 3.7 are shown in FIG. 7. FIG. 7 shows the results of measurements by means of continuous-flow cytometry of HLA-DR1-restricted human T-cells (PD2) which have been stained with fluorescent-labelled peptide-loaded HLA-DR1 tetramers.

3.8 Study of Compounds that Likewise Act Via the Deep P1 Pocket

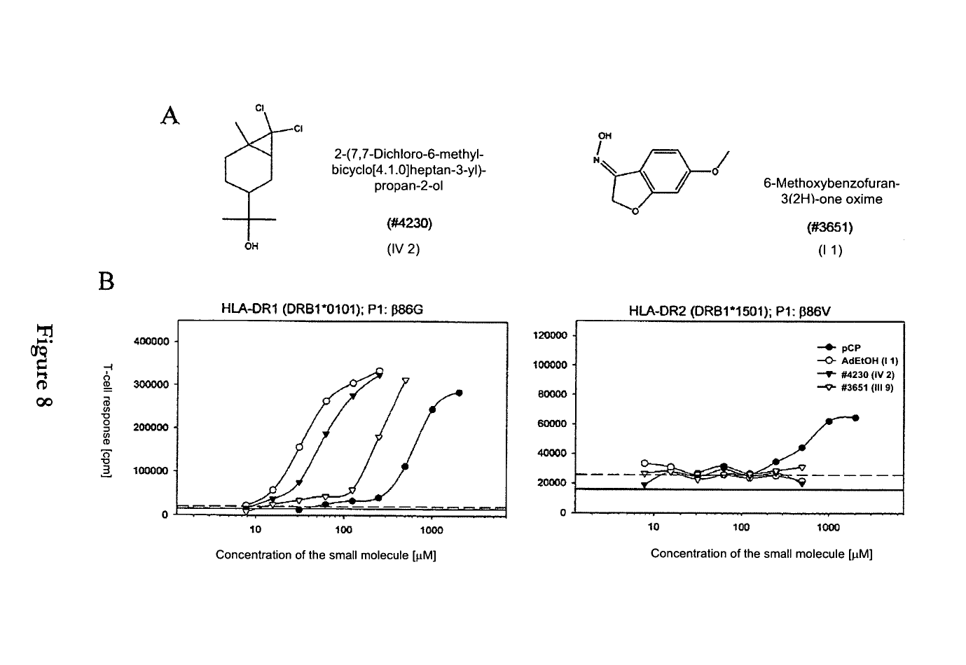

In addition to the adamantyl compounds, further compounds that exhibit the same allele specificity as the adamantyl compounds have been identified by the above-described methods, 2-(7,7-dichloro-6-methyl-bicyclo[4.1.0]heptan-3-yl)-propan-2-ol (#4230) and 6-methoxybenzofuran-3(2H)-one oxime (#3651). Despite a very different chemical structure, #4230 at least exhibits a spatial structure which is very similar to that of the adamantyl group. In the case of these compounds determined in addition, the activity and allele specificity were tested with the HLA-DR1-restricted HA306-318-specific EvHA/X5 T-cells and the HLA-DR2-restricted MBP86-100-specific 08073 T-cells. On HLA-DR1, #4230 exhibits a catalytic activity that corresponds approximately to that of adamantylethanol. #3651, on the other hand, is weaker but still exhibits higher activity than pCP. On HLA-DR2, both compounds exhibit no activity, for which reason they, similarly to the adamantyl compounds, presumably mediate their catalytic activity by the binding to the deep P1 pocket. The results of Comparison Test 3.8 are shown in FIG. 8.

3.9 Study of the Activity of Various Catalytically Active Phenol/Aniline Compounds

In the present comparison test, the catalytic activity of the example substances was demonstrated by means of the accelerated loading of soluble HLA-DR1 molecules with HA306-318. A comparison of the dose/activity curves shows that all the compounds lie within a very similar activity range, DCC and pHDP in particular exhibiting slightly increased activity. The results are shown in FIG. 9.

3.10 Catalysis of HLA-DR Loading

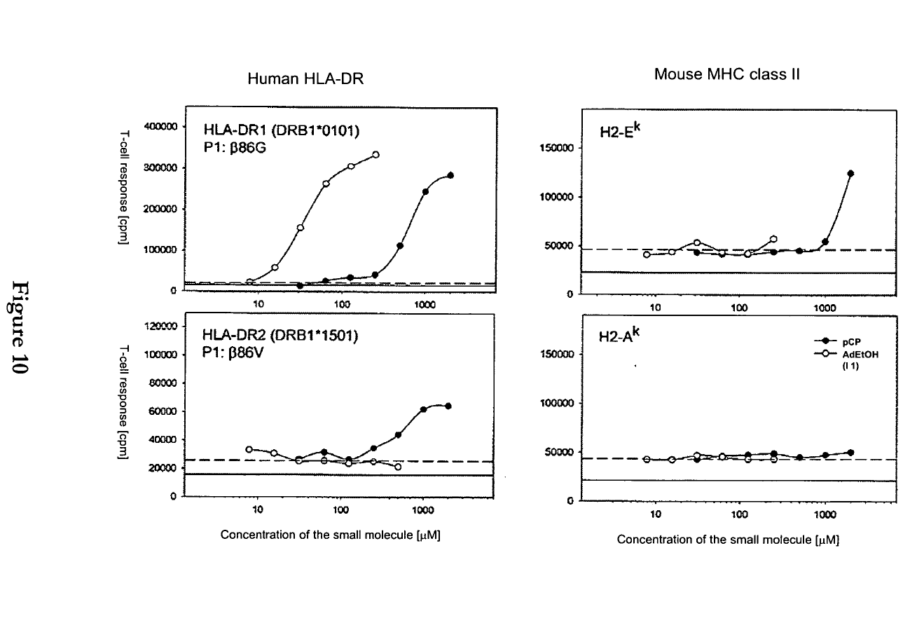

In the following, the catalysis of HLA loading by pCP was determined for the purposes of comparison. To this end, dose-activity curves of adamantylethanol (AdEtOH) and of pCP were determined by means of T-cell assays, as described in Comparison Test 3.3. These dose-activity curves show the activity of the two catalysts on HLA-DR molecules with a deep or flat P1 pocket {β86G and β86V} as well as on the mouse MHC class II molecules H2-Ek and H2-Ak. By means of the present test it was possible to show that the catalysis of HLA-DR binding is independent of the depth of the P1 pocket. The results are shown in FIG. 10.

3.11 Catalytic Activity of Thiophene Compounds

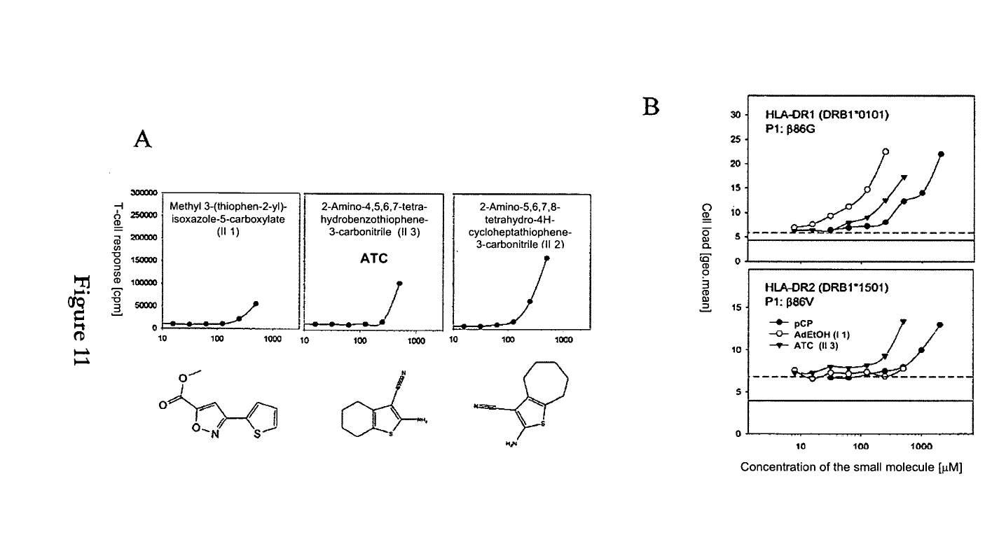

In order to investigate the catalytic activity of various catalytically active thiophene compounds, the activity of three example compounds was determined by way of example by means of the dose/activity curves of the stimulation of HLA-DR1-restricted EvHA/X5 T-cells. The test was carried out as described under Comparison Test 3.3. The results are shown in FIG. 11. As in the case of the active phenol/aniline compounds, the activity is here too evidently independent of the β86 dimorphism of the HLA-DR molecules. As shown in this example, the peptide loading both of fibroblasts that express either HLA-DR1 (DRB1*0101; β86G) or HLA-DR2 (DRB1*1501; β86V) is therefore catalysed by the compound ATC. Accordingly, the depth of the P1 pocket evidently plays no part in the case of the thiophene compounds either. The catalytic activity of the active compounds of this compound class identified hitherto is stronger than that of pCP.

3.12 Catalytic Activity of Specific Catalytically Active Compounds

The experimental data were acquired in an MHC loading assay with soluble HLA-DR1 (DRB1*0101) prepared by recombinant methods. To this end, the MHC molecules were incubated for about one hour with biotinylated HA306-318 in the presence of titrated amounts of small molecules (in various concentrations). The amount of peptide/MHC complexes formed within this time was then determined by means of an ELISA assay (capture antibody: anti-HLA-DR; detection: fluorescent-labelled streptavidin). The activities of the individual compounds were grouped on the basis of the dose/activity curves obtained thereby.

3.13 Measurement of the Enhancement of the DC-Mediated T-Cell Response by AdEtOH

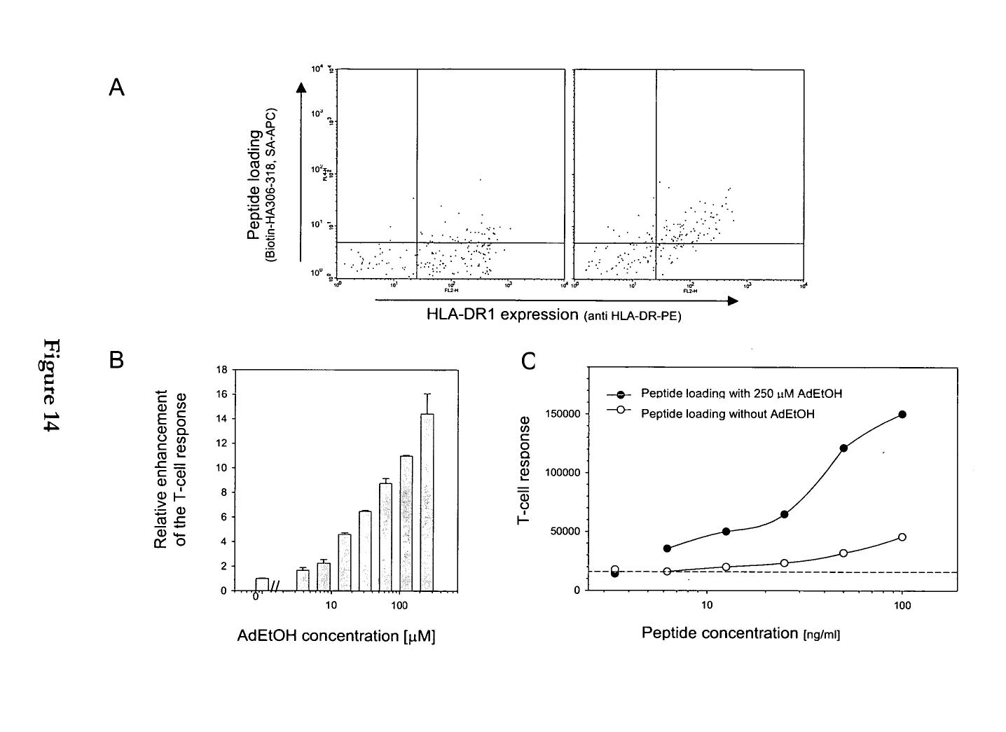

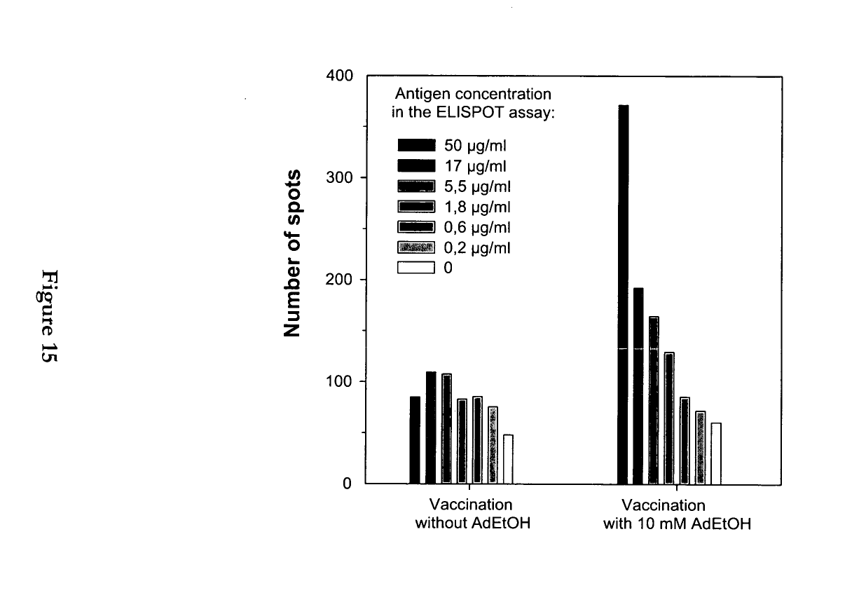

Dendritic cells (ICs) are one of the most important sub-groups of “professional” antigen-presenting cells. Therefore, in the present example, DCs from HLA-DR1 (DRB1*0101)-transgenic mice were used, the DCs being loaded for 4 hours with biotinylated HA306-318 peptide. Then, in order to determine the peptide load, the cells were stained with streptavidin-APC (SA-APC) and also with anti-HLA-DR and analysed by continuous-flow cytometry (FIG. 14A).

3.14 Use of Vaccination Additives and Use for Immunisation

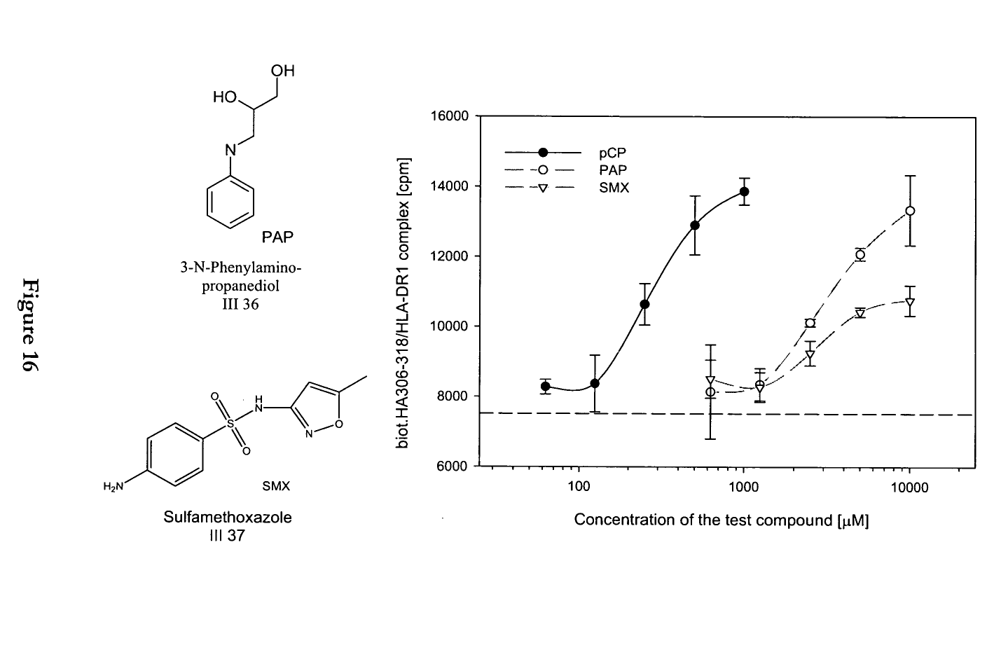

In this example, HLA-DR1 (DRB1*0101) transgenic mice were immunised with 10 mg of a domain of the ABL protein prepared by recombinant methods. The immunisation was carried out by subcutaneous administration of an emulsion of the antigen in complete Freund's adjuvant, which in the case of one group was additionally supplemented with 10 mM AdEtOH. The immune response triggered by the vaccination was then checked 12 days later by means of an ELISPOT assay. To this end, in each case about 10⁶ lymph node cells/well were transferred to culture plates coated with membrane filters and restimulated with titrated amounts of the antigen. Activation of a cell leads to the release of cytokines, such as, for example, IFN-g, which can be detected on the membrane by means of corresponding antibodies (see FIG. 15).

3.15 Use of a Method of MHC Loading for Identifying Immunotoxic Compounds

To this end, soluble HLA-DR1 (DRB1*0101) prepared by recombinant methods was incubated for 4 hours with biotinylated HA306-318 peptide in the presence of the amounts of the test substances indicated (in FIG. 16), and then the peptide/MHC complexes that formed were detected by means of ELISA (capture antibody: anti-HLA-DR; detection with fluorescent- or enzyme-labelled streptavidin). The results are shown in FIG. 16 or in the corresponding description relating to the figure.

### 4. Advantages of the Invention

Within the scope of this invention, methods for changing the load state of MHC molecules with ligands using compounds of formulae I, IA, II, III and IV1 to IV3 have been developed, which methods, compared with the use of the already known catalyst pCP for example for adamantane ethanol (AdaEtOH), dichlorophenylhydroxyiminoacetamide (DPHIA) and 4-hydroxybiphenyl (4-HBP), catalyse an increase in the load at concentrations above 0.025 mM. It is also possible within the scope of this invention to achieve a decrease in the load of MHC molecules with ligands, optionally until the ligands have been removed completely, and, alternatively, to replace ligands of MHC molecules using the compounds of formulae I, IA, II, III and IV1 to IV3. The compounds of formulae I, IA, II, III and IV1 to IV3 (catalysts) used in the methods according to the invention lead to a change in the conformation of the MHC molecules from a closed, non-receptive conformation to an open, receptive conformation, which for the first time permits the loading or replacement of the ligands of the MHC molecules. In comparison with pCP, a marked improvement has been achieved by means of the catalysts used according to the invention in the concentration of catalytically active compounds required to change the conformation. The catalysts used according to the invention accordingly permit higher activities, which permit the use of such compounds in animal experiments.

Furthermore, by means of the compounds of formulae I, IA, II, III and IV1 to IV3 used within the scope of this invention, efficient possibilities for triggering tumour-specific, pathogen-specific or autoreactive immune responses have been provided for the first time, as well as possibilities for the treatment of disorders or conditions associated with various pathologically excessive or absent immune reactions. Furthermore, by the provision of a vaccine or of a pharmaceutical composition, the treatment of cancer, infectious diseases, autoimmune disorders, or the attenuation of aggressive immune reactions, is possible.

1. Method for changing the load state of MHC molecules with ligands, comprising the following steps:
a) providing a composition containing MHC molecules; and
b) adding a catalyst selected from a compound of formula I or IA having the following structure: 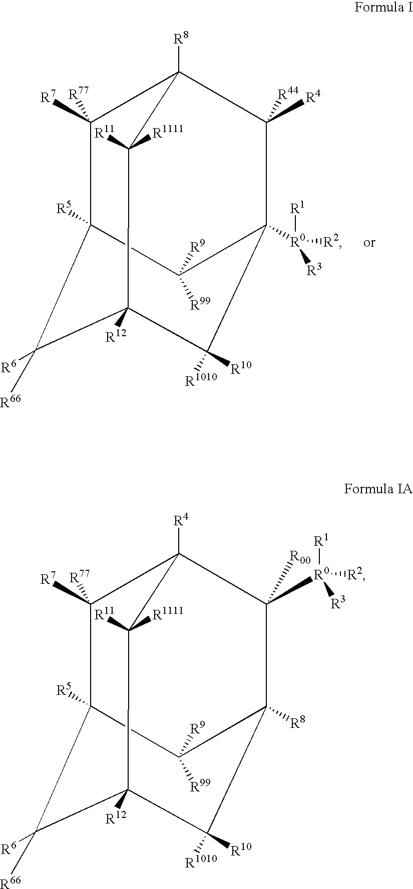
wherein:
R⁰, R⁰⁰, R¹, R², R³, R⁴, R⁵, R⁶, R⁷, R⁸, R⁹, R¹⁰, R¹¹, R¹², R⁴⁴, R⁶⁶, R⁷⁷, R⁹⁹, R¹⁰¹⁰ and R¹¹¹¹ can be a bond or are selected independently of one another from a group consisting of:
H, O, S, N,
OH, OR¹³,
SH, SO, SO₂, SO₂R¹³, SO₃, HSO₃, SR¹³, SR¹³R¹⁴, S(CH₂)ₙR¹³, S(CHₙ)R¹³; S(CH₂)ₙ(CH)ₙR¹³, S(CH₂)ₙ(CH)ₙR¹³,
NH, NH₂, NHNH₂, NHR¹³, NR¹³R¹⁴, NO, NO₂, NOH, NOR¹³,
X, CX₃, CHX₂, CH₂X, CR¹³X₂, CR₂¹³X, CR₃¹³, wherein X=halogen,
CN, CO, COR¹³, COOH, COOR¹³,
CH₃, (CH₂)ₙCH₃; (CH)ₙCH₃; (CH₂)ₙR¹³, (CH)ₙR¹³, (CH)ₙ(CH₂)ₙCH₃; (CH₂)ₙ(CH)ₙCH₃; (CH)ₙ(CH₂)ₙR¹³; (CH₂)ₙ(CH)ₙR¹³; C(R¹³)C(R¹⁴)CH₃, C(R¹³)(CH₂)ₙR¹⁴, (CH₂)ₙR¹³, (CH)ₙ(OH)R¹³; (CH₂)ₙ(OH)R¹³; (CH)ₙ(OH)CH₃; (CH₂)ₙ(OH)CH₃; OCH₃, O(CH₂)ₙCH₃, O(CH)ₙCH₃, O(CH₂)ₙR¹³, O(CH)ₙR¹³, O(CH)ₙ(CH₂)ₙR¹³, O(CH₂)ₙ(CH)ₙR¹³, (CH₂)ₙOCH₃, (CH)ₙOCH₃, (CH₂)ₙOR¹³, (CH)ₙOR¹³, (CH)ₙ(CH₂)ₙOR¹³, (CH₂)ₙ(CH)ₙOR¹³, (CH)ₙ(OH)CH₃; (CH₂)ₙ(OH)CH₃; (CH)ₙ(OH)R¹³; (CH₂)ₙ(OH)R¹³; (CH₂)ₙCH₂X; (CH)ₙCH₂X; (CH₂)ₙCH₂X; (CH₂)ₙX, (CH)ₙX, (CH)ₙ(CH₂)ₙCH₂X; (CH₂)ₙ(CH)ₙCH₂X; (CH)ₙ(CH₂)ₙX; (CH₂)ₙ(CH)ₙX; OCH₂X, O(CH₂)ₙCH₂X, O(CH)ₙCH₂X, O(CH₂)ₙX, O(CH)ₙX, O(CH)ₙ(CH₂)ₙX, O(CH₂)ₙ(CH)ₙX, (CH₂)ₙOCH₃, (CH)ₙOCH₂X, (CH₂)ₙNHR¹³, (CH₂)ₙNHOR¹³, (CH₂)ₙNHCOR¹³, (CH₂)ₙN(R¹³)CO, N(R¹³)(CH₂)ₙR¹⁴, N(R¹³)(CH)ₙR¹⁴, N(R¹³)(CH)ₙ(CH₂)ₙR¹⁴, N(R¹³)(CH₂)ₙ(CH)ₙR¹⁴, N(R¹³)COR¹⁴, N(R¹³)COOR¹⁴, CONH₂, CONHCH₃, C₃H₆OH, C(NH₂)(CH₂)ₙ(OH), OCONH(CH₂)ₙCH₃; OCONH(CH)ₙCH₃; OCONH(CH)ₙ(CH₂)ₙCH₃; OCONH(CH₂)ₙ(CH)ₙCH₃; (CH)ₙOR¹³, (CH₂)ₙOR¹³, C₆N₂H₅, C₆H₄(NHCOCH₃), C₆H₄SO₂NH, (CNNHC(CONHNH₂)CH₂), and C₆N₂H₇,
wherein n=from 1 to 30, and
R¹³ and R¹⁴ are selected independently of one another from a group consisting of
H, O, S, N,
OH, OR¹⁵,
SH, SO, SO₂, SO₃, HSO₃, SR¹⁵, SR¹⁵R¹⁶, S(CH₂)ₙR¹⁵, S(CHₙ)R¹⁵; S(CH₂)ₙ(CH)ₙR¹⁵, S(CH₂)ₙ(CH)ₙR¹⁵,
NH, NH₂, NHNH₂, NHR¹⁵, NR¹⁵R¹⁶, NO, NO₂, NOH, NOR¹⁵,
X, CX₃, CHX₂, CH₂X, CR¹⁵X₂, CR₂¹⁵X, CR₃¹⁵, wherein X=halogen,
CN, CO, COR¹⁵, COOH, COR¹⁵, COOR¹⁵,
CH₃, (CH₂)ₙCH₃; (CH)ₙCH₃; (CH₂)ₙR¹⁵, (CH)ₙR¹⁵, (CH)ₙ(CH₂)ₙCH₃; (CH₂)ₙ(CH)ₙCH₃; (CH)ₙ(CH₂)ₙR¹⁵; (CH₂)ₙ(CH)ₙR¹⁵; OCH₃, O(CH₂)ₙCH₃, O(CH)ₙCH₃, O(CH₂)ₙR¹⁵, O(CH)NR¹⁵, O(CH)ₙ(CH₂)ₙR¹⁵, O(CH₂)ₙ(CH)ₙR¹⁵, (CH₂)ₙOCH₃, (CH)ₙOCH₃, (CH₂)ₙOR¹⁵, (CH)NOR¹⁵, (CH)ₙ(CH₂)ₙOR¹⁵, (CH₂)ₙ(CH)ₙOR¹⁵, (CH)ₙ(OH)CH₃; (CH₂)ₙ(OH)CH₃; (CH)ₙ(OH)R¹⁵; (CH₂)ₙ(OH)R¹⁵; (CH₂)ₙCH₂X; (CH)ₙCH₂X; (CH₂)ₙCH₂X; (CH₂)ₙX, (CH)ₙX, (CH)ₙ(CH₂)ₙCH₂X; (CH₂)ₙ(CH)ₙCH₂X; (CH)ₙ(CH₂)ₙX; (CH₂)ₙ(CH)ₙX; OCH₂X, O(CH₂)ₙCH₂X, O(CH)ₙCH₂X, O(CH₂)ₙX, O(CH)ₙX, O(CH)ₙ(CH₂)ₙX, O(CH₂)ₙ(CH)ₙX, (CH₂)ₙOCH₃, (CH)ₙOCH₂X, (CH₂)ₙNHR¹⁵, (CH₂)ₙNHOR¹⁵, (CH₂)ₙNHCOR¹⁵, NR¹⁵(CH₂)ₙR¹⁶, NR¹⁵(CH)ₙR¹⁶, NR¹⁵(CH)ₙ(CH₂)ₙR¹⁶, NR¹⁵(CH₂)ₙ(CH)ₙR¹⁶, OCONH(CH₂)ₙCH₃; OCONH(CH)ₙCH₃; OCONH(CH)ₙ(CH₂)ₙCH₃; OCONH(CH₂)ₙ(CH)ₙCH₃; (CH)ₙOR¹⁵, (CH₂)ₙOR¹³, C₆N₂H₅, C₆H₄(NHCOCH₃), C₆H₄SO₂NH, (CNNHC(CONHNH₂)CH₂), adamantane, triazole, tetrazole, pyrazole, and oxazole;
wherein n=from 1 to 30, and
R¹⁵ and R¹⁶ are selected independently of one another from a group consisting of
H, O, S, N,
OH,
SH, SO, SO₂, SO₃, HSO₃,
NH, NH₂, NHNH₂, NO, NO₂, NHNH₂, NOH,
X, CX₃, CHX₂, CH₂X, wherein X=halogen,
CN, CO, COOH,
CH₃, (CH₂)ₙCH₃; (CH)ₙCH₃; (CH)ₙ(CH₂)ₙCH₃; (CH₂)ₙ(CH)ₙCH₃; OCH₃, O(CH₂)ₙCH₃, O(CH)ₙCH₃, (CH₂)ₙOCH₃, (CH)ₙOCH₃, (CH)ₙ(OH)CH₃; (CH₂)ₙ(OH)CH₃; (CH₂)ₙCH₂X; (CH)ₙCH₂X; (CH₂)ₙCH₂X; (CH₂)ₙX, (CH)ₙX,
(CH)ₙ(CH₂)ₙCH₂X; (CH₂)ₙ(CH)ₙCH₂X; (CH)ₙ(CH₂)ₙX; (CH₂)ₙ(CH)ₙX; OCH₂X, O(CH₂)ₙCH₂X, O(CH)ₙCH₂X, O(CH₂)ₙX, O(CH)ₙX, O(CH)ₙ(CH₂)ₙX, O(CH₂)ₙ(CH)ₙX, (CH₂)ₙOCH₃, (CH)ₙOCH₂X, OCONH(CH₂)ₙCH₃; OCONH(CH)ₙCH₃; OCONH(CH)ₙ(CH₂)ₙCH₃; OCONH(CH₂)ₙ(CH)ₙCH₃; C₆N₂H₅, C₆H₄(NHCOCH₃), C₆H₄SO₂NH, (CNNHC(CONHNH₂)CH₂), adamantane, triazole, tetrazole, pyrazole, and oxazole;
and/or
R⁰, R⁰⁰, R¹, R², R³, R⁴, R⁵, R⁶, R⁷, R⁸, R⁹, R¹⁰, R¹¹, R¹², R⁴⁴, R⁶⁶, R⁷⁷, R⁹⁹, R¹⁰¹⁰ and R¹¹¹¹ are selected independently of one another from a group consisting of a branched or unbranched C₁-C₃₀-alkyl, C₁-C₃₀-alkenyl, C₁-C₃₀-heteroalkyl, C₁-C₃₀-heteroalkenyl, C₁-C₃₀-alkoxy, C₁-C₃₀-alkenoxy, C₁-C₃₀, C₃-C₈-cycloalkyl, C₃-C₈-cycloalkenyl, C₅-C₃₀-aryl, C₅-C₃₀-heteroaryl, arylalkyl, arylalkenyl, C₅₋₂₀-aryloxy, heteroarylalkyl, heteroarylalkenyl, heterocycloalkyl, heterocycloalkenyl, carboxamido, acylamino, amidino, heteroaryloxy residue, adamantane, triazole, tetrazole, pyrazole, toluene, aniline, benzaldehyde, anisole, benzonitrile, phenol, acetophenone, benzoic acid, xylene, styrene, naphthalene, anthracene, phenanthrene, naphthalene, anthracene, phenanthrene, benzpyrene, pyridine, pyrimidine, purine, pyrrolidine, tetrahydrofuran, tetrahydrothiophene, tetrahydropyran, piperidine, pyrrole, furan, thiophene, pyridine, quinoline, indole, pyrimidine, pyrazine, purine, imidazole, pteridine, acridine, chromane, chromene, coumarin (chromen-2-one), and oxazole,
c) changing the load state of the MHC molecules; and
d) isolating the MHC molecules whose load state has been changed.

2. Method for changing the load state of MHC molecules with ligands, comprising the following steps:
a) providing a composition containing MHC molecules; and
b) adding a catalyst selected from a compound of formula II having the following structure: 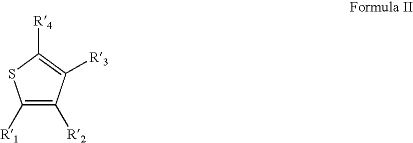
wherein:
R^{1′}, R^{2′}, R^{3′} and R^{4′} can be a bond or are selected independently of one another from a group consisting of:
H, O, S, N,
OH, OR^{13′}, SH, SO, SO₂, SO₂R^{13′}, SO₃, HSO₃, SR^{13′}, SR^{13′}R^{14′},
X, CX₃, CHX₂, CH₂X, CR^{13′}X₂, CR₂^{13′}X, CR₃^{13′} wherein X=halogen,
CN, CO, COOH, COOR¹³,
NH, NH₂, NHR^{13′}, NR^{13′}R^{14′}, NO, NO₂, NOH, NOR^{13′},
CH₃, (CH₂)ₙCH₃, (CH)ₙCH₃, (CH₂)ₙR^{13′}, (CH)ₙR^{13′}, OCH₃, O(CH₂)ₙ, O(CH₂)ₙCH₃, O(CH₂)ₙR^{13′}, (CH₂)ₙOH, (CH)ₙOH, (CH₂)ₙ(CH)ₙCH₃, (CH)ₙ(CH₂)ₙCH₃, (CH₂)ₙ(CH)ₙR^{13′}, (CH)ₙ(CH₂)ₙR^{13′}, —(C₃HNO)—CHX₂, (C₃HNO)—COOR^{13′}, —(C₃HNO)—CHR^{13′}R^{14′},
wherein n=from 1 to 30, and
R^{13′} and R^{14′} are selected independently of one another from a group consisting of
H, O, S, N,
OH, OR^{15′}, SH, SO, SO₂, SO₃, HSO₃, SR^{15′}, SR^{15′}R^{16′},
X, CX₃, CHX₂, CH₂X, CR^{15′}X₂, CR₂^{15′}X, CR₃^{15′} wherein X=halogen,
CN, CO, COOH, COOR^{15′},
NH, NH₂, NHR^{15′}, NR^{15′}R^{16′}, NO, NO₂, NOH, NOR^{15′},
CH₃, (CH₂)ₙCH₃, (CH)ₙCH₃, (CH₂)ₙR^{15′}, (CH)ₙR^{15′}, OCH₃, O(CH₂)ₙ, O(CH₂)ₙCH₃, O(CH₂)ₙR^{15′}, (CH₂)ₙOH, (CH)ₙOH, (CH₂)ₙ(CH)ₙCH₃, (CH)ₙ(CH₂)ₙCH₃, (CH₂)ₙ(CH)ₙR^{15′}, (CH)ₙ(CH₂)ₙR^{15′}, —(C₃HNO)—CHX₂, —(C₃HNO)—CHR^{15′}R^{16′},
wherein n=from 1 to 30,
R^{15′} and R^{16′} are selected independently of one another from a group consisting of
H, O, S, N,
OH, SH, SO, SO₂, SO₃, HSO₃,
X, CX₃, CHX₂, CH₂X, wherein X=halogen,
CN, CO, COOH,
NH, NH₂, NO, NO₂, NOH,
CH₃, (CH₂)ₙCH₃, (CH)ₙCH₃, OCH₃, O(CH₂)ₙ, O(CH₂)ₙCH₃, (CH₂)ₙOH, (CH)ₙOH, (CH₂)ₙ(CH)ₙCH₃, (CH)ₙ(CH₂)ₙCH₃, —(C₃HNO)—CHX₂, wherein n=from 1 to 30,
and/or
R^{1′}, R^{2′}, R^{3′} and R^{4′} are selected independently of one another from a group consisting of a branched or unbranched C₁-C₃₀-alkyl, C₁-C₃₀-alkenyl, C₁-C₃₀-heteroalkyl, C₁-C₃₀-heteroalkenyl, C₁-C₃₀-alkoxy, C₁-C₃₀-alkenoxy, C₁-C₃₀-acyl, C₃-C₈-cycloalkyl, C₃-C₈-cycloalkenyl, C₅-C₃₀-aryl, C₅-C₃₀-heteroaryl, arylalkyl, arylalkenyl, C₅₋₃₀-aryloxy, heteroarylalkyl, heteroarylalkenyl, heterocycloalkyl, heterocycloalkenyl, carboxamido, acylamino, amidino, adamantyl residue, heteroaryloxy residue, toluene, aniline, benzaldehyde, anisole, benzonitrile, phenol, acetophenone, benzoic acid, xylene, styrene, naphthalene, anthracene, phenanthrene, naphthalene, anthracene, phenanthrene, benzpyrene, pyridine, pyrimidine, purine, pyrrolidine, tetrahydrofuran, tetrahydrothiophene, tetrahydropyran, piperidine, pyrrole, furan, thiophene, pyridine, quinoline, indole, pyrimidine, pyrazine, purine, imidazole, pteridine, acridine, chromane, chromene, and coumarin (chromen-2-one), adamantane, pyrazole, diazole, tetrazole, triazole,
and/or
R^{1′} and R^{2′} together can form a bridged structure selected from a branched or unbranched C₁-C₈-alkyl, C₁-C₈-alkenyl, C₁-C₈-heteroalkyl, C₁-C₈-heteroalkenyl, C₁-C₈-alkoxy, C₁-C₈-alkenoxy, C₁-C₈-acyl, C₃-C₈-cycloalkyl, C₃-C₈-cycloalkenyl, C₅-C₈-aryl, C₅-C₈-heteroaryl, arylalkyl, arylalkenyl, C₅₋₈-aryloxy, heteroarylalkyl, heteroarylalkenyl, heterocycloalkyl, heterocycloalkenyl, carboxamido, acylamino, amidino, adamantyl residue, heteroaryloxy residue, toluene, aniline, benzaldehyde, anisole, benzonitrile, phenol, acetophenone, benzoic acid, xylene, styrene, naphthalene, anthracene, phenanthrene, naphthalene, anthracene, phenanthrene, benzpyrene, pyridine, pyrimidine, purine, pyrrolidine, tetrahydrofuran, tetrahydrothiophene, tetrahydropyran, piperidine, pyrrole, furan, thiophene, pyridine, quinoline, indole, pyrimidine, pyrazine, purine, imidazole, pteridine, acridine, chromane, chromene, and coumarin (chromen-2-one), adamantane, pyrazole, diazole, tetrazole, triazole, wherein one or two substituents selected from R^{1′} and R^{2′} as described hereinbefore can occur independently of one another at each individual atom of the bridged structure, preferably from 1 to 12 atoms,
c) changing the load state of the MI-IC molecules; and
d) isolating the MHC molecules whose load state has been changed.

3. Method for changing the load state of MHC molecules with ligands, comprising the following steps:
a) providing a composition containing MHC molecules; and
b) adding a catalyst selected from a compound of formula III having the following structure: 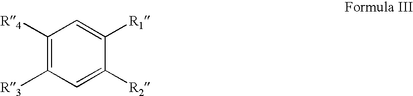
wherein:
R^{1″} and R^{2″} can be a bond or are selected independently of one another from a group consisting of:
H, O, S, N,
OH, OR^{13″}, SH, SO, SO₂, SO₂R^{13″}, SO₃, HSO₃, SR^{13″}, SR^{13″}R^{14″}, S(CH₂)ₙ(CH₄N);
X, CX₃, CHX₂, CH₂X, CR^{13″}X₂, CR₂^{13″}X wherein X=halogen,
CN, CO, COOH, COOCH₃, COOR¹³,
NH, NH₂, NHR^{13″}, NR^{13″}R^{14″}, NR^{13″}(CO)R^{14″}; NO, NO₂, NOH, CHNOH, NOR^{13″}, CH₃, (CH₂)ₙ, (CH₂)ₙCH₃, (CH₂)ₙR^{13″}, (CH)ₙCR^{13″}R^{14″}, OCH₃, O(CH₂)ₙ, O(CH₂)ₙCH₃; O(CH₂)ₙR^{13″}, (CH₂)ₙOH, C₄H₂O(CH₃); (C₃H₂NO)(R^{13″}), (O(CH₂)ₙCH(R^{13″})S(O₂)); (C(CH₃)(CH₂)ₙNHC(O)S), ((CH₂)ₙN(CH₂)ₙC(R^{13″})S), (CHC(R^{13″})N(R^{14″})NC(R^{13″}), NR^{13″}(CH₂)ₙR^{14″}, and (C₂H₃N₂O(NR^{13″}R^{14″}), wherein n=from 1 to 30, and
R^{13″} and R^{14″} are selected independently of one another from a group consisting of
H, O, S, N,
OH, OR^{15″}, SH, SO, SO₂, SO₃, HSO₃, SR^{15″}, SR^{15″}R^{15″}, SC(CX₃)XCOOR^{15″},
X, CX₃, CHX₂, CH₂X, CR^{15″}X₂, CR₂^{15″}X wherein X=halogen,
CN, CO, COOH, COOCH₃, COOR^{15″},
NH, NH₂, NHR^{13″}, NR^{15″}R^{16″}, NO, NO₂, NOH, NOR^{15″},
CH₃, (CH₂)ₙ, (CH₂)ₙCH₃, (CH₂)ₙR^{15″}, OCH₃, O(CH₂)ₙ, O(CH₂)ₙCH₃; O(CH₂)ₙR^{15″}, (CH₂)ₙOH, C₆H₄CH₃, C₆H₉, C₃H₅N₂O₂, (C₃H₂NS)(R^{15″}), and (N(R^{15″}C₃HNO(R^{16″})), CH(R^{15″})(CH₂)ₙR^{16″},
wherein n=from 1 to 30, and
R^{15″} and R^{16″} are selected independently of one another from a group consisting of
H, O, S, N,
OH, SH, SO, SO₂, SO₃, HSO₃,
X, CX₃, CHX₂, CH₂X, wherein X=halogen,
CN, CO, COOH, COOCH₃,
NH, NH₂, NO, NO₂, NOH,
CH₃, (CH₂)ₙ, (CH₂)ₙCH₃, OCH₃, O(CH₂)ₙ, O(CH₂)ₙCH₃; and (CH₂)ₙOH,
wherein n=from 1 to 30,
and/or
R^{1″} and R^{2″} are selected independently of one another from a group consisting of a branched or unbranched C₁-C₃₀-alkyl, C₁-C₃₀-alkenyl, C₁-C₃₀-heteroalkyl, C₁-C₃₀-heteroalkenyl, C₁-C₃₀-alkoxy, C₁-C₃₀-alkenoxy, C₁-C₃₀-acyl, C₃-C₈-cycloalkyl, C₃-C₈-cycloalkenyl, C₅-C₃₀-aryl, C₅-C₃₀-heteroaryl, arylalkyl, arylalkenyl, C₅₋₃₀-aryloxy, heteroarylalkyl, heteroarylalkenyl, heterocycloalkyl, heterocycloalkenyl, carboxamido, acylamino, amidino, adamantyl or heteroaryloxy residue; toluene, aniline, benzaldehyde, anisole, benzonitrile, phenol, acetophenone, benzoic acid, xylene, styrene, naphthalene, anthracene, phenanthrene, naphthalene, anthracene, phenanthrene, benzpyrene, pyridine, pyrimidine, purine, pyrrolidine, tetrahydrofuran, tetrahydrothiophene, tetrahydropyran, piperidine, pyrrole, furan, thiophene, pyridine, quinoline, indole, pyrimidine, pyrazine, purine, imidazole, pteridine, acridine, chromane, chromene, and coumarin (chromen-2-one), diazole, tetrazole, pyrazole, C₃H₂S₂O, saturated or unsaturated C₆₋₈-lactone, and succinimide,
and/or
R^{3″} and R^{4″} are as defined for R^{1″} and R^{2″} or can be a bond or are selected independently of one another from a group consisting of:
H, O, S, N,
SH, SO, SO₂, SO₃, HSO₃, SR^{13″}, SR^{13″}R^{14″},
X, in particular Br, CX₃, CHX₂, CH₂X, CR^{13″}X₂, CR₂^{13″}X, CR₃^{13″} wherein X=halogen,
CN, CO, COOH, COOR^{13″},
NH, NH₂, NHR^{13″}, NR^{13″}R^{14″}, NO, NO₂, NOH, NOR^{13″},
CH₃, (CH₂)ₙ, (CH₂)ₙCH₃, (CH₂)ₙR^{13″}, OCH₃, O(CH₂)ₙ, O(CH₂)ₙCH₃; O(CH₂)ₙR^{13″}, (CH₂)ₙOH, C₆H₁₀OH, SO₂CF₃, S(CCH(CH₃)N(OH)NC(CH₃)), (CNONC)NHCH₂(N₄CH), NHC(O)(C₄H₂O(CH₃)), CH₂(C₂N₂H₅(CO)₂), SCFCF₃COOCH₃, SCH₂(C₂NSH(NH₂)), C₃N₂H₃, C(CH₃)C(O)NHC(O)CH₂, C(CH₃)CH₂NHC(O)S, C₆H₅, NHC(O)CHNOH, S(CH₂)₂(C₅H₄N), CHC(CN)(COOCH₃), C₃H₄N, S(C(CH₃)NHNC(CH₃)), NH(C₆H₃N₂O), C₆H₄S(O)₂NH, C₆H₄NHC(O)CH₃, NHC(O)CHNOH, and adamantyl;
wherein n=from 1 to 30, and
R^{13″} and R^{14″} are selected independently of one another from
H, O, S, N,
SH, SO, SO₂, SO₃, HSO₃, SR^{15″}, SR^{15″}R^{16″},
X, in particular Br, CX₃, CHX₂, CH₂X, CR^{15″}X₂, CR₂^{15″}X, CR₃^{15″} wherein X=halogen,
CN, CO, COOH, COOR^{15″},
NH, NH₂, NHR^{15″}, NR^{15″}R^{16″}, NO, NO₂, NOH, NOR^{15″},
CH₃, (CH₂)ₙCH₃, (CH₂)ₙR^{15″}, OCH₃, O(CH₂)ₙ, O(CH₂)ₙCH₃; O(CH₂)ₙR^{15″}, (CH₂)ₙOH, C₆H₁₀OH, SO₂CF₃, S(CCH(CH₃)N(OH)NC(CH₃)), (CNONC)NHCH₂(N₄CH), NHC(O)(C₄H₂O(CH₃)), CH₂(C₂N₂H₅(CO)₂), SCFCF₃COOCH₃, SCH₂(C₂NSH(NH₂)), C₃N₂H₃, C(CH₃)C(O)NHC(O)CH₂, C(CH₃)CH₂NHC(O)S, C₆H₅, NHC(O)CHNOH, S(CH₂)₂(C₅H₄N), CHC(CN)(COOCH₃), C₃H₄N, S(C(CH₃)NHNC(CH₃)), NH(C₆H₃N₂O), C₆H₄S(O)₂NH, C₆H₄NHC(O)CH₃, NHC(O)CHNOH, adamantyl;
wherein n=from 1 to 30, and
R^{15″} and R^{16″} are selected independently of one another from a group consisting of
H, O, S, N,
SH, SO, SO₂, SO₃, HSO₃,
X, in particular Br, CX₃, CHX₂, CH₂X, wherein X=halogen,
CN, CO, COOH, COOCH₃,
NH, NH₂, NO, NO₂, NOH,
CH₃, (CH₂)ₙCH₃, OCH₃, O(CH₂)ₙ, O(CH₂)ₙCH₃; (CH₂)ₙOH, C₆H₁₀OH, SO₂CF₃, S(CCH(CH₃)N(OH)NC(CH₃)), (CNONC)NHCH₂(N₄CH), NHC(O)(C₄H₂O(CH₃)), CH₂(C₂N₂H₅(CO)₂), SCFCF₃COOCH₃, SCH₂(C₂NSH(NH₂)), C₃N₂H₃, C(CH₃)C(O)NHC(O)CH₂, C(CH₃)CH₂NHC(O)S, C₆H₅, NHC(O)CHNOH, S(CH₂)₂(C₅H₄N), CHC(CN)(COOCH₃), C₃H₄N, S(C(CH₃)NHNC(CH₃)), NH(C₆H₃N₂O), C₆H₄S(O)₂NH, C₆H₄NHC(O)CH₃, NHC(O)CHNOH, and adamantyl;
wherein n=from 1 to 30,
and/or
R^{3″} and R^{4″} can be selected independently of one another from a group consisting of a branched or unbranched C₁-C₃₀-alkyl, C₁-C₃₀-alkenyl, C₁-C₃₀-heteroalkyl, C₁-C₃₀-heteroalkenyl, C₁-C₃₀-alkoxy, C₁-C₃₀-alkenoxy, C₁-C₃₀-acyl, C₃-C₈-cycloalkyl, C₃-C₈-cycloalkenyl, C₅-C₃₀-aryl, C₅-C₃₀-heteroaryl, arylalkyl, arylalkenyl, C₅₋₃₀-aryloxy, heteroarylalkyl, heteroarylalkenyl, heterocycloalkyl, heterocycloalkenyl, carboxamido residue, acylamino residue, amidino residue, adamantyl residue, heteroaryloxy residue, toluene, aniline, benzaldehyde, anisole, benzonitrile, phenol, acetophenone, benzoic acid, xylene, styrene, naphthalene, anthracene, phenanthrene, naphthalene, anthracene, phenanthrene, benzpyrene, pyridine, pyrimidine, purine, pyrrolidine, tetrahydrofuran, tetrahydrothiophene, tetrahydropyran, piperidine, pyrrole, furan, thiophene, pyridine, quinoline, indole, pyrimidine, pyrazine, purine, imidazole, pteridine, acridine, chromane, chromene, and coumarin (chromen-2-one), diazole, tetrazole, pyrazole, C₃H₂S₂O, saturated or unsaturated C₆₋₈-lactone, and succinimide;
and/or
R^{3″} and R^{4″} together can form a bridged structure selected from a branched or unbranched C₁-C₈-alkyl, C₁-C₈-alkenyl, C₁-C₈-heteroalkyl, C₁-C₈-heteroalkenyl, C₁-C₈-alkoxy, C₁-C₈-alkenoxy, C₁-C₈-acyl, C₃-C₈-cycloalkyl,
C₃-C₈-cycloalkenyl, C₅-C₈-aryl, C₅-C₈-heteroaryl, arylalkyl, arylalkenyl, C₅₋₈-aryloxy, heteroarylalkyl, heteroarylalkenyl, heterocycloalkyl, heterocycloalkenyl, carboxamido, acylamino, amidino, adamantyl residue, heteroaryloxy residue, toluene, aniline, benzaldehyde, anisole, benzonitrile, phenol, acetophenone, benzoic acid, xylene, styrene, naphthalene, anthracene, phenanthrene, naphthalene, anthracene, phenanthrene, benzpyrene, pyridine, pyrimidine, purine, pyrrolidine, tetrahydrofuran, tetrahydrothiophene, tetrahydropyran, piperidine, pyrrole, furan, thiophene, pyridine, quinoline, indole, pyrimidine, pyrazine, purine, imidazole, pteridine, acridine, chromane, chromene, and coumarin (chromen-2-one), diazole, tetrazole, pyrazole, C₃H₂S₂O, saturated or unsaturated C₆₋₈-lactone, and succinimide, wherein one or two substituents selected from R^{1″} and R^{2″} can occur independently of one another at each individual atom of the bridged structure, preferably from 1 to 12 atoms,
c) changing the load state of the MHC molecules; and
d) isolating the MHC molecules whose load state has been changed.

4. Method according to claim 1, characterised in that
R⁰, R⁰⁰, R¹, R², R³, R⁴, R⁵, R⁶, R⁷, R⁸, R⁹, R¹⁰, R¹¹, R¹², R⁴⁴, R⁶⁶, R⁷⁷, R⁹⁹, R¹⁰¹⁰ and R¹¹¹¹ can be selected together or independently of one another from a group consisting of:
H, O, S, N,
OH, OR¹³,
SH, SO, SO₂, SO₂R¹³, SO₃, HSO₃, SR¹³, SR¹³R¹⁴, S(CH₂)ₙR¹³, S(CHₙ)R¹³; S(CH₂)ₙ(CH)ₙR¹³, S(CH₂)ₙ(CH)ₙR¹³,
NH, NH₂, NHNH₂, NHR¹³, NR¹³R¹⁴, NO, NO₂, NOH, NOR¹³,
X, CX₃, CHX₂, CH₂X, CR¹³X₂, CR₂¹³X, CR₃¹³, wherein X=halogen,
CN, CO, COR¹³, COOH, COOR¹³,
CH₃, (CH₂)ₙCH₃; (CH)ₙCH₃; (CH₂)ₙR¹³, (CH)ₙR¹³, (CH)ₙ(CH₂)ₙCH₃; (CH₂)ₙ(CH)ₙCH₃; (CH)ₙ(CH₂)ₙR¹³; (CH₂)ₙ(CH)ₙR¹³; C(R¹³)C(R¹⁴)CH₃, C(R¹³)(CH₂)ₙR¹⁴, (CH₂)ₙR¹³, (CH)ₙ(OH)R¹³; (CH₂)ₙ(OH)R¹³; (CH)ₙ(OH)CH₃; (CH₂)ₙ(OH)CH₃; OCH₃,
O(CH₂)ₙCH₃, O(CH)ₙCH₃, O(CH₂)ₙR¹³, O(CH)ₙR¹³, O(CH)ₙ(CH₂)ₙR¹³, O(CH₂)ₙ(CH)ₙR¹³, (CH₂)ₙOCH₃, (CH)ₙOCH₃, (CH₂)ₙOR¹³, (CH)ₙOR¹³, (CH)ₙ(CH₂)ₙOR¹³, (CH₂)ₙ(CH)ₙOR¹³, (CH)ₙ(OH)CH₃; (CH₂)ₙ(OH)CH₃; (CH)ₙ(OH)R¹³; (CH₂)ₙ(OH)R¹³; (CH₂)ₙCH₂X; (CH)ₙCH₂X; (CH₂)ₙCH₂X; (CH₂)ₙX, (CH)ₙX, (CH)ₙ(CH₂)ₙCH₂X; (CH₂)ₙ(CH)ₙCH₂X; (CH)ₙ(CH₂)ₙX; (CH₂)ₙ(CH)ₙX; OCH₂X, O(CH₂)ₙCH₂X, O(CH)ₙCH₂X, O(CH₂)ₙX, O(CH)ₙX, O(CH)ₙ(CH₂)ₙX, O(CH₂)ₙ(CH)ₙX, (CH₂)ₙOCH₃, (CH)ₙOCH₂X, (CH₂)ₙNHR¹³, (CH₂)ₙNHOR¹³, (CH₂)ₙNHCOR¹³, (CH₂)ₙN(R¹³)CO, N(R¹³)(CH₂)ₙR¹⁴, N(R¹³)(CH)ₙR¹⁴, N(R¹³)(CH)ₙ(CH₂)ₙR¹⁴, N(R¹³)(CH₂)ₙ(CH)ₙR¹⁴, N(R¹³)COR¹⁴, N(R¹³)COOR¹⁴, CONH₂, CONHCH₃, C₃H₆OH, C(NH₂)(CH₂)ₙ(OH), OCONH(CH₂)ₙCH₃; OCONH(CH)ₙCH₃; OCONH(CH)ₙ(CH₂)ₙCH₃; OCONH(CH₂)ₙ(CH)ₙCH₃; (CH)ₙOR¹³, (CH₂)ₙOR¹³, C₆N₂H₅, C₆H₄(NHCOCH₃), C₆H₄SO₂NH, and (CNNHC(CONHNH₂)CH₂), C₆N₂H₇,
wherein n=from 1 to 10, and
R¹³ and R¹⁴ are selected independently of one another from a group consisting of
H, O, S, N,
OH, OR¹⁵,
SH, SO, SO₂, SO₃, HSO₃, SR¹⁵, SR¹⁵R¹⁶, S(CH₂)ₙR¹⁵, S(CHₙ)R¹⁵; S(CH₂)ₙ(CH)ₙR¹⁵, S(CH₂)ₙ(CH)ₙR¹⁵,
NH, NH₂, NHNH₂, NHR¹⁵, NR¹⁵R¹⁶, NO, NO₂, NOH, NOR¹⁵,
X, CX₃, CHX₂, CH₂X, CR¹⁵X₂, CR₂¹⁵X, CR₃¹⁵, wherein X=halogen,
CN, CO, COR¹⁵, COOH, COR¹⁵, COOR¹⁵,
CH₃, (CH₂)ₙCH₃; (CH)ₙCH₃; (CH₂)ₙR¹⁵, (CH)ₙR¹⁵, (CH)ₙ(CH₂)ₙCH₃; (CH₂)ₙ(CH)ₙCH₃; (CH)ₙ(CH₂)ₙR¹⁵; (CH₂)ₙ(CH)ₙR¹⁵; OCH₃, O(CH₂)ₙCH₃, O(CH)ₙCH₃, O(CH₂)ₙR¹⁵, O(CH)ₙR¹⁵, O(CH)ₙ(CH₂)ₙR¹⁵, O(CH₂)ₙ(CH)ₙR¹⁵, (CH₂)ₙOCH₃, (CH)ₙOCH₃, (CH₂)ₙOR¹⁵, (CH)ₙOR¹⁵, (CH)ₙ(CH₂)ₙOR¹⁵, (CH₂)ₙ(CH)ₙOR¹⁵, (CH)ₙ(OH)CH₃; (CH₂)ₙ(OH)CH₃; (CH)ₙ(OH)R¹⁵; (CH₂)ₙ(OH)R¹⁵;
(CH₂)ₙCH₂X; (CH)ₙCH₂X; (CH₂)ₙCH₂X; (CH₂)ₙX, (CH)ₙX, (CH)ₙ(CH₂)ₙCH₂X; (CH₂)ₙ(CH)ₙCH₂X; (CH)ₙ(CH₂)ₙX; (CH₂)ₙ(CH)ₙX; OCH₂X, O(CH₂)ₙCH₂X, O(CH)ₙCH₂X, O(CH₂)ₙX, O(CH)ₙX, O(CH)ₙ(CH₂)ₙX, O(CH₂)ₙ(CH)ₙX, (CH₂)ₙOCH₃, (CH)ₙOCH₂X, (CH₂)ₙNHR¹⁵, (CH₂)ₙNHOR¹⁵, (CH₂)ₙNHCOR¹⁵, NR¹⁵(CH₂)ₙR¹⁶, NR¹⁵(CH)ₙR¹⁶, NR¹⁵(CH)ₙ(CH₂)ₙR¹⁶, NR¹⁵(CH₂)ₙ(CH)ₙR¹⁶, OCONH(CH₂)ₙCH₃; OCONH(CH)ₙCH₃; OCONH(CH)ₙ(CH₂)ₙCH₃; OCONH(CH₂)ₙ(CH)ₙCH₃; (CH)ₙOR¹⁵, (CH₂)ₙOR¹³, C₆N₂H₅, C₆H₄(NHCOCH₃), C₆H₄SO₂NH, (CNNHC(CONHNH₂)CH₂), adamantane, triazole, tetrazole, pyrazole, and oxazole;
wherein n=from 1 to 10, and
R¹⁵ and R¹⁶ are selected independently of one another from a group consisting of
H, O, S, N,
OH,
SH, SO, SO₂, SO₃, HSO₃,
NH, NH₂, NHNH₂, NO, NO₂, NHNH₂, NOH,
X, CX₃, CHX₂, CH₂X, wherein X=halogen,
CN, CO, COOH,
CH₃, (CH₂)ₙCH₃; (CH)ₙCH₃; (CH)ₙ(CH₂)ₙCH₃; (CH₂)ₙ(CH)ₙCH₃; OCH₃, O(CH₂)ₙCH₃, O(CH)ₙCH₃, (CH₂)ₙOCH₃, (CH)ₙOCH₃, (CH)ₙ(OH)CH₃; (CH₂)ₙ(OH)CH₃; (CH₂)ₙCH₂X; (CH)ₙCH₂X; (CH₂)ₙCH₂X; (CH₂)ₙX, (CH)ₙX, (CH)ₙ(CH₂)ₙCH₂X; (CH₂)ₙ(CH)ₙCH₂X; (CH)ₙ(CH₂)ₙX; (CH₂)ₙ(CH)ₙX; OCH₂X, O(CH₂)ₙCH₂X, O(CH)ₙCH₂X, O(CH₂)ₙX, O(CH)ₙX, O(CH)ₙ(CH₂)ₙX, O(CH₂)ₙ(CH)ₙX, (CH₂)ₙOCH₃, (CH)ₙOCH₂X, OCONH(CH₂)ₙCH₃; OCONH(CH)ₙCH₃; OCONH(CH)ₙ(CH₂)ₙCH₃; OCONH(CH₂)ₙ(CH)ₙCH₃; C₆N₂H₅, C₆H₄(NHCOCH₃), C₆H₄SO₂NH, (CNNHC(CONHNH₂)CH₂), adamantane, triazole, tetrazole, pyrazole, and oxazole.

5. Method according to claim 4, characterised in that the compound of formula I is selected from one of the following structures:

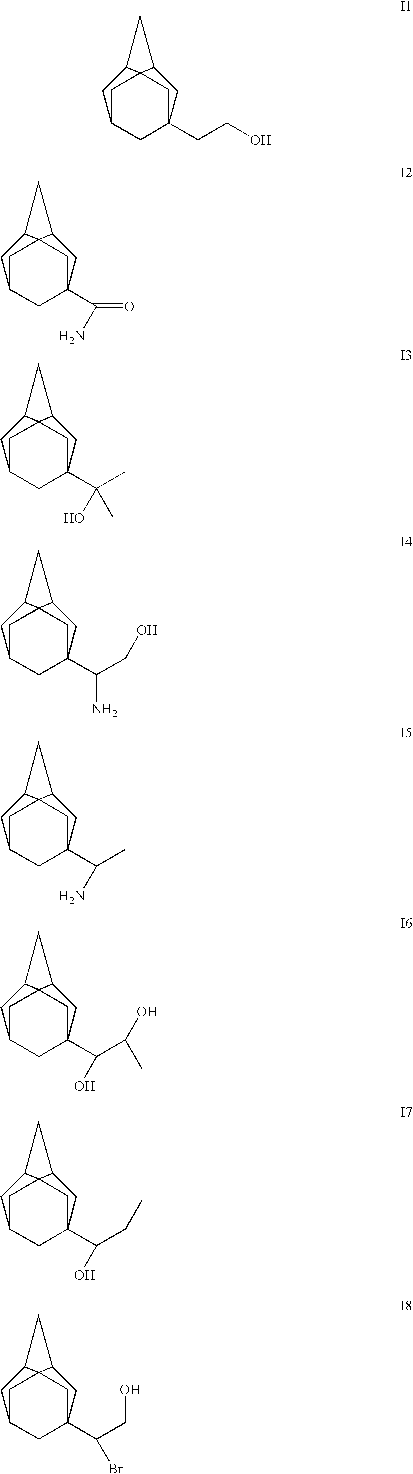

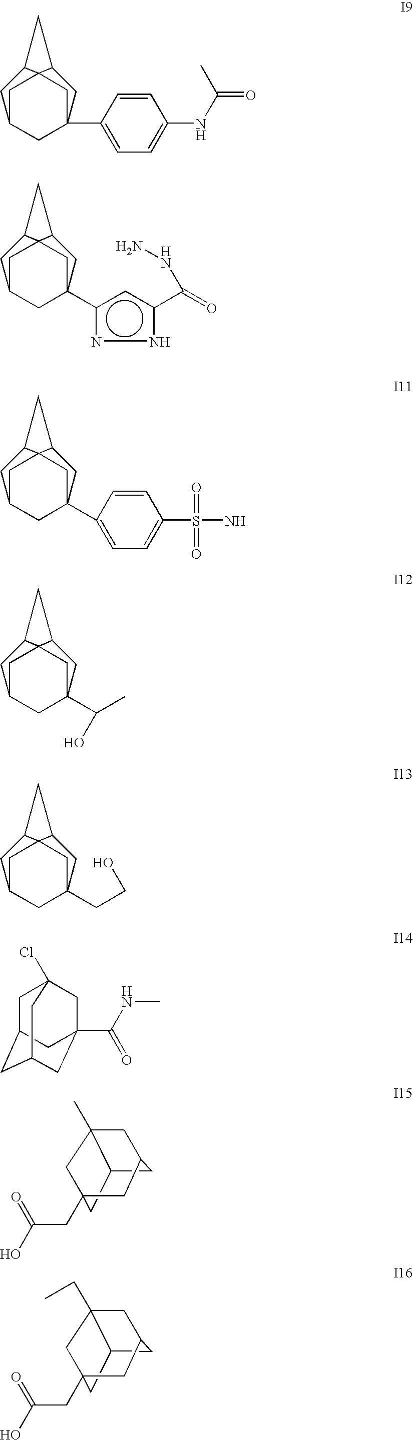

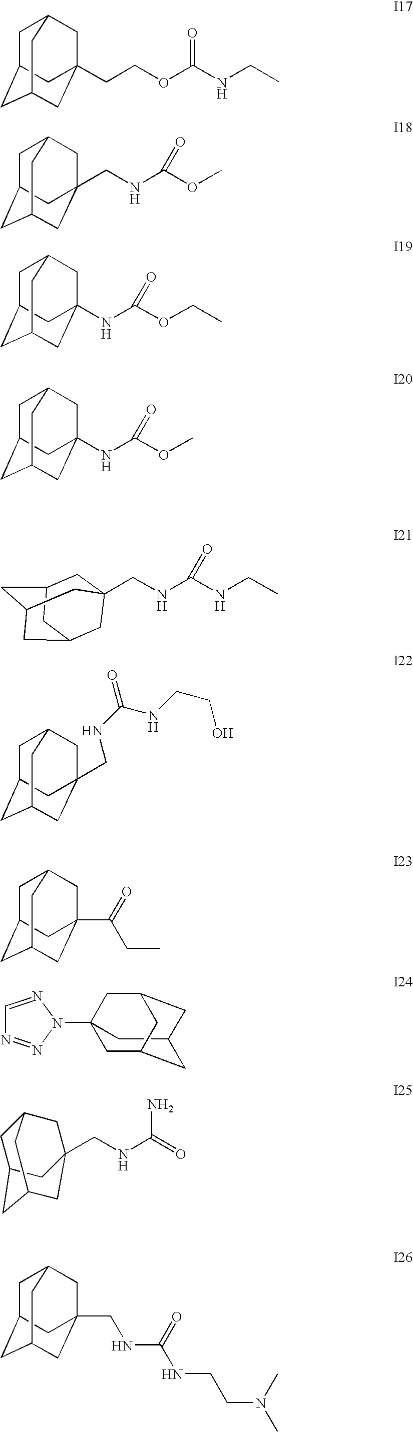

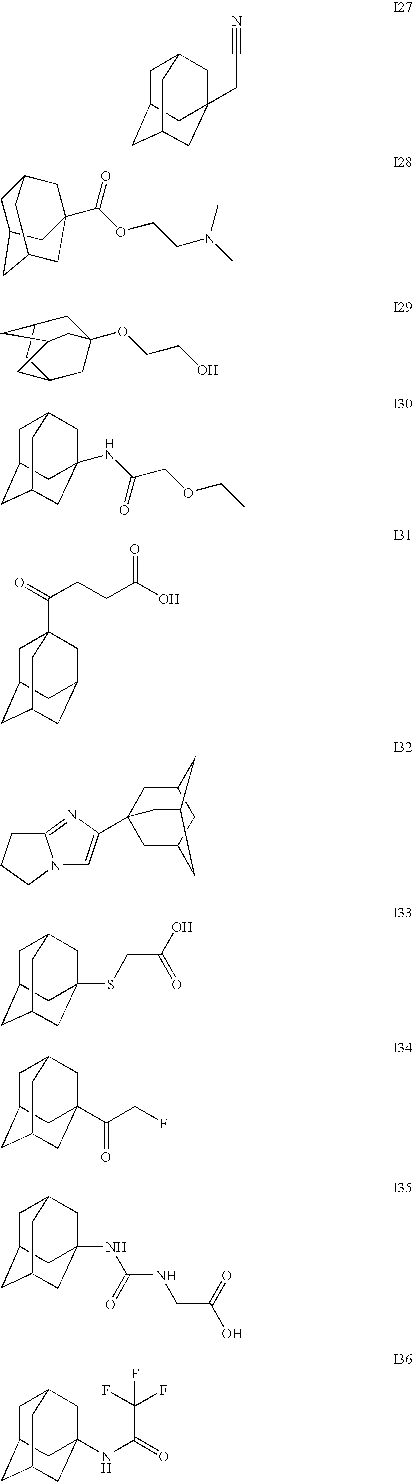

6. Method according to claim 2, characterised in that
R^{1′}, R^{2′}, R^{3′} or R^{4′} can be a bond or are selected together or independently of one another from a group consisting of:
H, O, S, N,
OH, OR^{13′}, SH, SO, SO₂, SO₂R^{13′}, SO₃, HSO₃, SR^{13′}, SR^{13′}R^{14′},
X, CX₃, CHX₂, CH₂X, CR^{13′}X₂, CR₂^{13′}X, CR₃^{13′} wherein X=halogen,
CN, CO, COOH, COOR^{13′},
NH, NH₂, NHR^{13′}, NR^{13′}R^{14′}, NO, NO₂, NOH, NOR^{13′},
CH₃, (CH₂)ₙCH₃, (CH)ₙCH₃, (CH₂)ₙR^{13′}, (CH)ₙR^{13′}, OCH₃, O(CH₂)ₙ, O(CH₂)ₙCH₃, O(CH₂)ₙR^{13′}, (CH₂)ₙOH, (CH)ₙOH, (CH₂)ₙ(CH)ₙCH₃, (CH)ₙ(CH₂)ₙCH₃, (CH₂)ₙ(CH)ₙR^{13′}, (CH)ₙ(CH₂)ₙR^{13′}, —(C₃HNO)—CHX₂, (C₃HNO)—COOR^{13′}, —(C₃HNO)—CHR^{13′}R^{14′},
wherein n=from 1 to 10, and
R^{13′} and R^{14′} are selected independently of one another from a group consisting of
H, O, S, N,
OH, OR^{15′}, SH, SO, SO₂, SO₃, HSO₃, SR^{15′}, SR^{15′}R^{16′},
X, CX₃, CHX₂, CH₂X, CR^{15′}X₂, CR₂^{15′}X, CR₃^{15′} wherein X=halogen,
CN, CO, COOH, COOR^{15′},
NH, NH₂, NHR^{15′}, NR^{15′}R^{16′}, NO, NO₂, NOH, NOR^{15′},
CH₃, (CH₂)ₙCH₃, (CH)ₙCH₃, (CH₂)ₙR^{15′}, (CH)ₙR^{15′}, OCH₃, O(CH₂)ₙ, O(CH₂)ₙCH₃, O(CH₂)ₙR^{15′}, (CH₂)ₙOH, (CH)ₙOH, (CH₂)ₙ(CH)ₙCH₃, (CH)ₙ(CH₂)ₙCH₃, (CH₂)ₙ(CH)ₙR^{15′}, (CH)ₙ(CH₂)ₙR^{15′}, —(C₃HNO)—CHX₂, —(C₃HNO)—CHR^{15′}R^{16′},
wherein n=from 1 to 10, and
R^{15′} and R^{16′} are selected independently of one another from a group consisting of
H, O, S, N,
OH, SH, SO, SO₂, SO₃, HSO₃,
X, CX₃, CHX₂, CH₂X, wherein X=halogen,
CN, CO, COOH,
NH, NH₂, NO, NO₂, NOH,
CH₃, (CH₂)ₙCH₃, (CH)ₙCH₃, OCH₃, O(CH₂)ₙ, O(CH₂)ₙCH₃, (CH₂)ₙOH, (CH)ₙOH, (CH₂)ₙ(CH)ₙCH₃, (CH)ₙ(CH₂)ₙCH₃, —(C₃HNO)—CHX₂, wherein n=from 1 to 10,
and/or
R^{1′} and R^{2′} together can form a bridged structure selected from a branched or unbranched C₁-C₈-alkyl, C₁-C₈-alkenyl, C₁-C₈-heteroalkyl, C₁-C₈-heteroalkenyl, C₁-C₈-alkoxy, C₁-C₈-alkenoxy, C₁-C₈-acyl, C₃-C₈-cycloalkyl, C₃-C₈-cycloalkenyl, C₅-C₈-aryl, C₅-C₈-heteroaryl, arylalkyl, arylalkenyl, C₅₋₈-aryloxy, heteroarylalkyl, heteroarylalkenyl, heterocycloalkyl, heterocycloalkenyl, carboxamido, acylamino, amidino, adamantyl residue, heteroaryloxy residue, toluene, aniline, benzaldehyde, anisole, benzonitrile, phenol, acetophenone, benzoic acid, xylene, styrene, naphthalene, anthracene, phenanthrene, naphthalene, anthracene, phenanthrene, benzpyrene, pyridine, pyrimidine, purine, pyrrolidine, tetrahydrofuran, tetrahydrothiophene, tetrahydropyran, piperidine, pyrrole, furan, thiophene, pyridine, quinoline, indole, pyrimidine, pyrazine, purine, imidazole, pteridine, acridine, chromane, chromene, and coumarin (chromen-2-one),
adamantane, pyrazole, diazole, tetrazole, triazole, wherein one or two substituents selected from R^{1′} and R^{2′} as defined hereinbefore can occur independently of one another at each individual atom of the bridged structure, preferably from 1 to 12 atoms.

7. Method according to claim 6, characterised in that the compound of formula II is selected from one of the following structures:

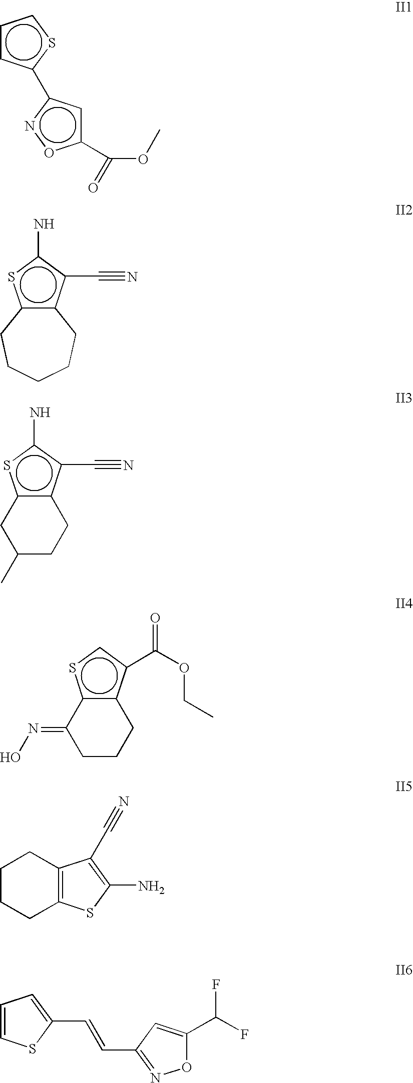

8. Method according to claim 3, characterised in that
R^{1″} and R^{2″} can be a bond or are selected independently of one another from a group consisting of:
H, O, S, N,
OH, OR^{13″}, SH, SO, SO₂, SO₂R^{13″}, SO₃, HSO₃, SR^{13″}, SR^{13″}R^{14″}, S(CH₂)ₙ(CH₄N);
X, CX₃, CHX₂, CH₂X, CR^{13″}X₂, CR₂^{13″}X wherein X=halogen,
CN, CO, COOH, COOCH₃, COOR^{13″},
NH, NH₂, NHR^{13″}, NR^{13″}R^{14″}, NR^{13″}(CO)R^{14″}; NO, NO₂, NOH, CHNOH, NOR^{13″},
CH₃, (CH₂)ₙ, (CH₂)ₙCH₃, (CH₂)ₙR^{13″}, (CH)ₙCR^{13″}R^{14″}, OCH₃, O(CH₂)ₙ, O(CH₂)ₙCH₃; O(CH₂)ₙR^{13″}, (CH₂)ₙOH, C₄H₂O(CH₃); (C₃H₂NO)R^{13″}), (O(CH₂)ₙCH(R^{13″})S(O₂)); (C(CH₃)(CH₂)ₙNHC(O)S), ((CH2)ₙN(CH₂)ₙC(R^{13″})S), (CHC(R^{13″})N(R^{14″})NC(R^{13″}), NR^{13″}(CH₂)ₙR^{14″}, and (C₂H₃N₂O(NR^{13″}R^{14″})),
wherein n=from 1 to 10, and
R^{13″} and R^{14″} are selected independently of one another from a group consisting of
H, O, S, N,
OH, OR^{15″}, SH, SO, SO₂, SO₃, HSO₃, SR^{15″}, SR^{15″}R^{15″}, SC(CX₃)XCOOR^{15″},
X, CX₃, CHX₂, CH₂X, CR^{15″}X₂, CR₂^{15″}X wherein X=halogen,
CN, CO, COOH, COOCH₃, COOR^{15″},
NH, NH₂, NHR^{13″}, NR^{15″}R^{16″}, NO, NO₂, NOH, NOR^{15″},
CH₃, (CH₂)ₙ, (CH₂)ₙCH₃, (CH₂)ₙR^{15″}, OCH₃, O(CH₂)ₙ, O(CH₂)ₙCH₃; O(CH₂)ₙR^{15″}, (CH₂)ₙOH, C₆H₄CH₃, C₆H₉, C₃H₅N₂O₂, (C₃H₂NS)(R^{15″}), and (N(R^{15″}C₃HNO(R^{16″})), CH(R^{15″})(CH₂)ₙR^{16″},
wherein n=from 1 to 10, and
R^{15″} and R^{16″} are selected independently of one another from a group consisting of
H, O, S, N,
OH, SH, SO, SO₂, SO₃, HSO₃,
X, CX₃, CHX₂, CH₂X, wherein X=halogen,
CN, CO, COOH, COOCH₃,
NH, NH₂, NO, NO₂, NOH,
CH₃, (CH₂)ₙ, (CH₂)ₙCH₃, OCH₃, O(CH₂)ₙ, O(CH₂)ₙCH₃; and (CH₂)ₙOH, wherein n=from 1 to 10,
and/or
R^{1″} and R^{2″} are selected independently of one another from a group consisting of a branched or unbranched C₁-C₃₀-alkyl, C₁-C₃₀-alkenyl, C₁-C₃₀-heteroalkyl, C₁-C₃₀-heteroalkenyl, C₁-C₃₀-alkoxy, C₁-C₃₀-alkenoxy, C₁-C₃₀-acyl, C₃-C₈-cycloalkyl, C₃-C₈-cycloalkenyl, C₅-C₃₀-aryl, C₅-C₃₀-heteroaryl, arylalkyl, arylalkenyl, C₅₋₃₀-aryloxy, heteroarylalkyl, heteroarylalkenyl, heterocycloalkyl, heterocycloalkenyl, carboxamido, acylamino, amidino, adamantyl or heteroaryloxy residue; toluene, aniline, benzaldehyde, anisole, benzonitrile, phenol, acetophenone, benzoic acid, xylene, styrene, naphthalene, anthracene, phenanthrene, naphthalene, anthracene, phenanthrene, benzpyrene, pyridine, pyrimidine, purine, pyrrolidine, tetrahydrofuran, tetrahydrothiophene, tetrahydropyran, piperidine, pyrrole, furan, thiophene, pyridine, quinoline, indole, pyrimidine, pyrazine, purine, imidazole, pteridine, acridine, chromane, chromene, and coumarin (chromen-2-one), diazole, tetrazole, pyrazole, C₃H₂S₂O, saturated or unsaturated C₆₋₈-lactone, and succinimide,
and/or
R^{3″} and R^{4″} are as defined for R^{1″} or R^{2″} or can be a bond or are selected independently of one another from a group consisting of:
H, O, S, N,
SH, SO, SO₂, SO₃, HSO₃, SR^{13″}, SR^{13″}R^{14″},
X, in particular Br, CX₃, CHX₂, CH₂X, CR^{13″}X₂, CR₂^{13″}X, CR₃^{13″} wherein X=halogen,
CN, CO, COOH, COOR^{13″},
NH, NH₂, NHR^{13″}, NR^{13″}R^{14″}, NO, NO₂, NOH, NOR^{13″},
CH₃, (CH₂)ₙ, (CH₂)ₙCH₃, (CH₂)ₙR^{13″}, OCH₃, O(CH₂)ₙ, O(CH₂)ₙCH₃; O(CH₂)ₙR^{13″}, (CH₂)ₙOH, C₆H₁₀OH, SO₂CF₃, S(CCH(CH₃)N(OH)NC(CH₃)), (CNONC)NHCH₂(N₄CH), NHC(O)(C₄H₂O(CH₃)), CH₂(C₂N₂H₅(CO)₂), SCFCF₃COOCH₃, SCH₂(C₂NSH(NH₂)), C₃N₂H₃, C(CH₃)C(O)NHC(O)CH₂, C(CH₃)CH₂NHC(O)S, C₆H₅, NHC(O)CHNOH, S(CH₂)₂(C₅H₄N), CHC(CN)(COOCH₃), C₃H₄N, S(C(CH₃)NHNC(CH₃)), NH(C₆H₃N₂O), C₆H₄S(O)₂NH, C₆H₄NHC(O)CH₃, NHC(O)CHNOH, and adamantyl; wherein n=from 1 to 10, and
R^{13″} and R^{14″} are selected independently of one another from
H, O, S, N,
SH, SO, SO₂, SO₃, HSO₃, SR^{15″}, SR^{15″}R^{16″},
X, in particular Br, CX₃, CHX₂, CH₂X, CR^{15″}X₂, CR₂^{15″}X, CR₃^{15″} wherein X=halogen,
CN, CO, COOH, COOR^{15″},
NH, NH₂, NHR^{15″}, NR^{15″}R^{16″}, NO, NO₂, NOH, NOR^{15″},
CH₃, (CH₂)ₙCH₃, (CH₂)ₙR^{15″}, OCH₃, O(CH₂)ₙ, O(CH₂)ₙCH₃; O(CH₂)ₙR^{15″}, (CH₂)ₙOH, C₆H₁₀OH, SO₂CF₃, S(CCH(CH₃)N(OH)NC(CH₃)), (CNONC)NHCH₂(N₄CH), NHC(O)(C₄H₂O(CH₃)), CH₂(C₂N₂H₅(CO)₂), SCFCF₃COOCH₃, SCH₂(C₂NSH(NH₂)), C₃N₂H₃, C(CH₃)C(O)NHC(O)CH₂, C(CH₃)CH₂NHC(O)S, C₆H₅, NHC(O)CHNOH, S(CH₂)₂(C₅H₄N), CHC(CN)(COOCH₃), C₃H₄N, S(C(CH₃)NHNC(CH₃)), NH(C₆H₃N₂O), C₆H₄S(O)₂NH, C₆H₄NHC(O)CH₃, NHC(O)CHNOH, adamantyl;
wherein n=from 1 to 10, and
R^{15″} and R^{16″} are selected independently of one another from a group consisting of
H, O, S, N,
SH, SO, SO₂, SO₃, HSO₃,
X, in particular Br, CX₃, CHX₂, CH₂X, wherein X=halogen,
CN, CO, COOH, COOCH₃,
NH, NH₂, NO, NO₂, NOH,
CH₃, (CH₂)ₙCH₃, OCH₃, O(CH₂)ₙ, O(CH₂)ₙCH₃; (CH₂)ₙOH, C₆H₁₀OH, SO₂CF₃, S(CCH(CH₃)N(OH)NC(CH₃)), (CNONC)NHCH₂(N₄CH), NHC(O)(C₄H₂O(CH₃)), CH₂(C₂N₂H₅(CO)₂), SCFCF₃COOCH₃, SCH₂(C₂NSH(NH₂)), C₃N₂H₃, C(CH₃)C(O)NHC(O)CH₂, C(CH₃)CH₂NHC(O)S, C₆H₅, NHC(O)CHNOH, S(CH₂)₂(C₅H₄N), CHC(CN)(COOCH₃), C₃H₄N, S(C(CH₃)NHNC(CH₃)), NH(C₆H₃N₂O), C₆H₄S(O)₂NH, C₆H₄NHC(O)CH₃, NHC(O)CHNOH, and adamantyl; wherein n=from 1 to 10,
and/or
R^{3″} and R^{4″} together can form a bridged structure selected from a branched or unbranched C₁-C₈-alkyl, C₁-C₈-alkenyl, C₁-C₈-heteroalkyl, C₁-C₈-heteroalkenyl, C₁-C₈-alkoxy, C₁-C₈-alkenoxy, C₁-C₈-acyl, C₃-C₈-cycloalkyl, C₃-C₈-cycloalkenyl, C₅-C₈-aryl, C₅-C₈-heteroaryl, arylalkyl, arylalkenyl, C₅₋₈-aryloxy, heteroarylalkyl, heteroarylalkenyl, heterocycloalkyl, heterocycloalkenyl, carboxamido, acylamino, amidino, adamantyl residue, heteroaryloxy residue, toluene, aniline, benzaldehyde, anisole, benzonitrile, phenol, acetophenone, benzoic acid, xylene, styrene, naphthalene, anthracene, phenanthrene, naphthalene, anthracene, phenanthrene, benzpyrene, pyridine, pyrimidine, purine, pyrrolidine, tetrahydrofuran, tetrahydrothiophene, tetrahydropyran, piperidine, pyrrole, furan, thiophene, pyridine, quinoline, indole, pyrimidine, pyrazine, purine, imidazole, pteridine, acridine, chromane, chromene, and coumarin (chromen-2-one), diazole, tetrazole, pyrazole, C₃H₂S₂O, saturated and unsaturated C₆₋₈-lactone, and succinimide, wherein one or two substituents selected from R^{1″} and R^{2″} can occur independently of one another at each individual atom of the bridged structure, preferably from 1 to 12 atoms.

9. Method according to claim 8, characterised in that the compound of formula III is selected from one of the following structures:

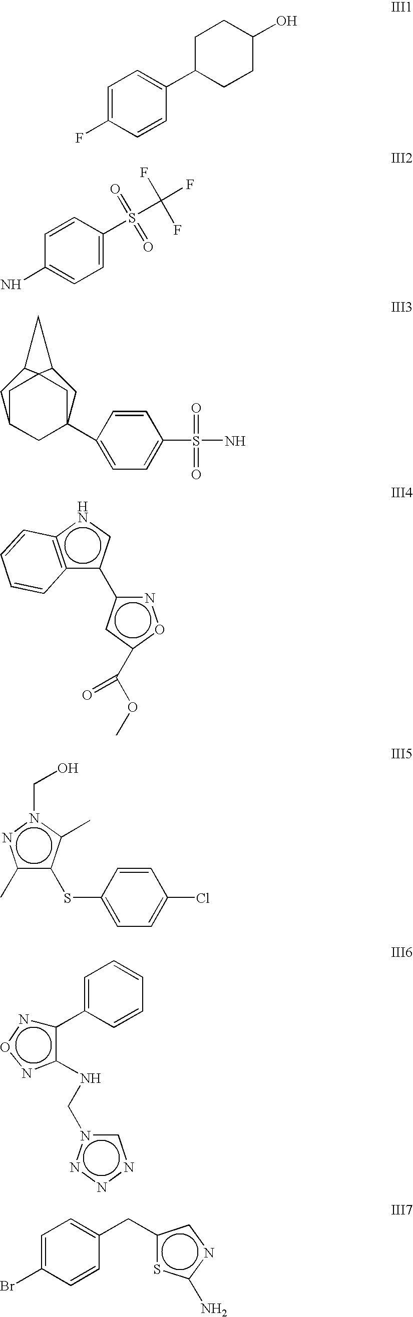

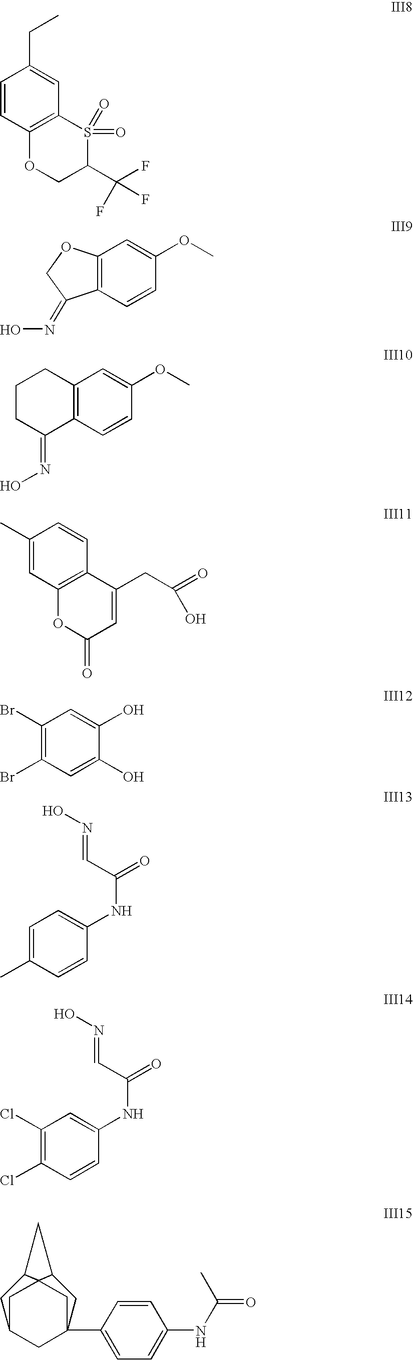

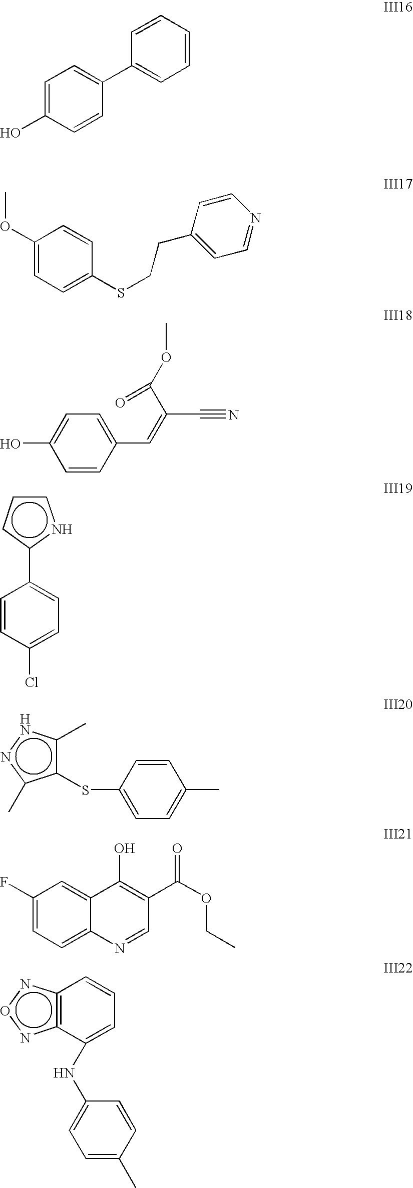

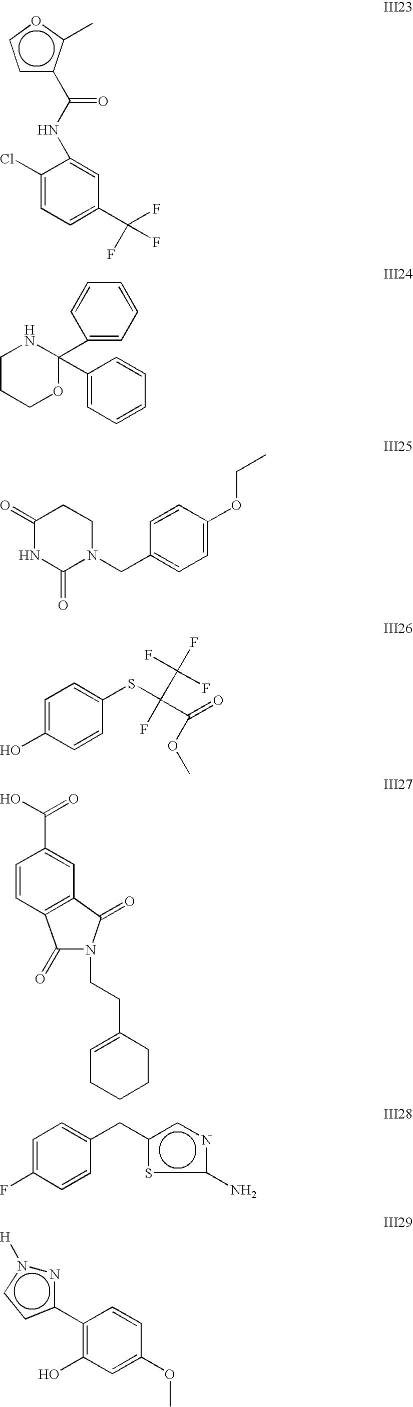

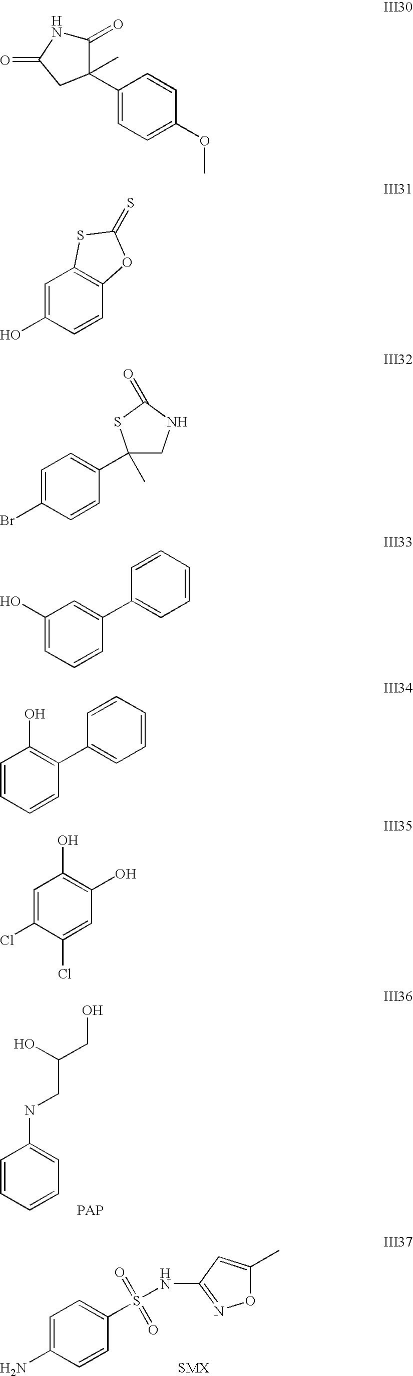

10. Method for changing the load state of MHC molecules with ligands, comprising the following steps:
a) providing a composition containing MHC molecules; and
b) adding a catalyst selected from a compound of formulae IV1 to IV3; 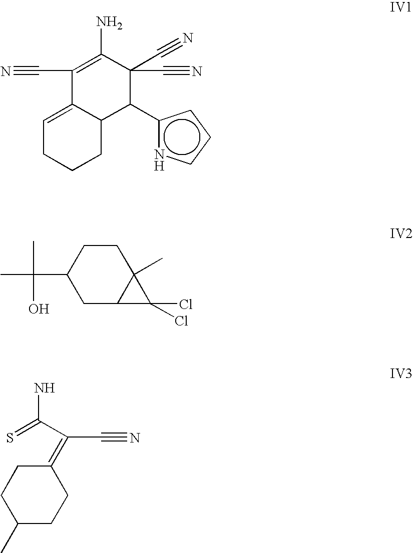
c) changing the load state of the MHC molecules; and
d) isolating the MHC molecules whose load state has been changed.

11. Method according to claim 1, characterised in that steps (a) and (b) are interchangeable.

12. Method according to claim 1, characterised in that the MHC molecules are MHC class I or II molecules.

13. Method according to claim 1, characterised in that the MHC molecules are loaded with ligands or are unloaded.

14. Method according to claim 1, characterised in that the ligand is selected from antigens, in particular tumour- or pathogen-specific antigens, tissue-specific self-antigens, antigens of autoreactive cells, peptide antigens and fragments of such peptide antigens, complete proteins, protein mixtures and/or complex protein mixtures.

15. Method according to claim 1, characterised in that the change in the load state of the MHC molecules in step (c) leads to the loading of unloaded MHC molecules with ligands.

16. Method according to claim 15, characterised in that the loading of the MHC molecules in step (c) is carried out by addition of potential ligands of MHC molecules.

17. Method according to claim 1, characterised in that the change in the load state of the MHC molecules leads in an alternative step (c′) to the replacement of ligands of loaded MHC molecules by different ligands.

18. Method according to claim 17, characterised in that the replacement of ligands of MHC molecules loaded with ligands in the alternative step (c′) comprises the following steps:
(i) decreasing the load of MHC molecules loaded with ligands;
(ii) adding different ligands of MHC molecules.

19. Method according to claim 15, characterised in that, in order to trigger tumour-specific, pathogen-specific or autoreactive immune responses, the loading of MHC molecules is increased with antigenic ligands.

20. Method according to claim 19, characterised in that, in order to trigger the immune responses, loading of antigen-presenting cells (APCs) is carried out.

21. Method according to claim 20, characterised in that the antigen-presenting cells are selected from endogenous or non-endogenous maturated and non-maturated dendritic cells, B-cells or macrophages or other antigen-presenting cells.

22. Method according to claim 1, characterised in that the change in the load state of the MHC molecules leads in an alternative step (c″) to a decrease in the load of MHC molecules loaded with ligands.

23. Method according to claim 22, characterised in that the decrease in the load of MHC molecules loaded with ligands in step (c″) is carried out by a washing step.

24. Method according to claim 22, characterised in that the decrease in the load of MHC molecules loaded with ligands in step (c″) leads to complete removal of the ligands.

25. Method according to claim 22, characterised in that a decrease in the load of MHC molecules loaded with antigens leads to the attenuation of aggressive immune reactions.

26. Method according to claim 1, characterised in that the change in the load state of MHC molecules is carried out at a binding pocket of an MHC molecule.

27. Method according to claim 26, characterised in that the binding pocket is a binding pocket of an MHC I molecule.

28. Method according to claim 27, characterised in that the peptide binding pocket of an MHC I molecule is selected from peptide binding pockets A, B, C, D, E or F.

29. Method according to claim 26, characterised in that the binding pocket is a peptide binding pocket of an MHC II molecule.

30. Method according to claim 29, characterised in that the binding pocket of an MHC II molecule is selected from peptide binding pockets P1, P3, P4, P6, P7 and P9.

31. Method according to claim 30, characterised in that the binding pocket is the binding pocket P1.

32. Screening method for seeking and identifying new antigens, for detecting specific cytotoxic T-cells or for monitoring a specific T-cell response, comprising the following steps:
a) providing a composition containing MHC molecules whose load state has been changed with ligands by a method according to claim 1; and
b) determining the interaction of these MHC molecules whose load state has been changed with ligands by a method according to claim 1, with a physiological binding partner of the MHC molecules by means of a biochemical or biophysical detection method.

33. Method according to claim 32, characterised in that the screening method includes in vitro T-cell assays, proliferation assays, ELISPOTS, ELISA methods, chromium-release assays and high-throughput screening methods (HTS).

34. MHC molecule obtainable by a method according to any one of claims 1, 2, 3 or 10.

35. (canceled)

36. (canceled)

37. (canceled)

38. (canceled)

39. (canceled)

40. (canceled)

41. (canceled)

42. (canceled)

43. (canceled)

44. Vaccine containing an MHC molecule loaded with ligands according to claim 34, and optionally a pharmaceutically acceptable carrier.

45. Vaccine containing a compound of formulae I, IA, II, III or IV1 to IV3 as defined in claims 1, 2, 3, or 10 together with ligands, and optionally a pharmaceutically acceptable carrier.

46. Vaccine according to claim 45, characterised in that the ligand is selected from antigens, in particular tumour- or pathogen-specific antigens, tissue-specific auto-antigens, peptide antigens and fragments of such peptide antigens, complete proteins, protein mixtures and/or complex protein mixtures.

47. Pharmaceutical composition containing an MHC molecule loaded with ligands according to claim 34, and optionally a pharmaceutically acceptable carrier.

48. Method of identifying substances having the property of changing the load state of MHC molecules, characterised in that (a) unloaded MHC molecules, in particular in solution or fixed to a surface, are provided, (b) a compound of formulae I, IA, II, III or IV1 to IV3 is added, (c) at the same time as or after step (b) ligands of the MHC molecule provided are added, and (d) the loading or binding of the MHC molecules with the ligands added according to step (c) is measured.

49. Method of identifying substances having the property of changing the load state of MHC molecules, characterised in that (a) MHC molecules loaded with ligands, in particular in solution or fixed to a surface, are provided, (b) a compound of formulae I, IA, II, III or IV1 to IV3 is added, and (c) the dissociation of the ligands from the MHC molecules is measured.

50. Method according to either claim 48 or claim 49, characterised in that the measurement is carried out kinetically, by surface plasmon resonance or by thermodynamic, in particular microcalorimetric, methods.
